(19) United States
(12) Patent Application Publication
(10) Pub. No.: US 2022/0326241 A1
(43) Pub. Date: Oct. 13, 2022

(54) PROTEOGENOMIC METHODS FOR DIAGNOSING CANCER

(71) Applicants: Baylor College of Medicine, Houston, TX (US); The Broad Institute, Inc., Cambridge, MA (US); The General Hospital Corporation, Boston, MA (US)

(72) Inventors: Matthew J. Ellis, Houston, TX (US); Bing Zhang, Houston, TX (US); Eric Jaehnig, Houston, TX (US); Steven Carr, Cambridge, MA (US); Shankha Satpathy, Cambridge, MA (US); Michael Gillette, Boston, MA (US)

(21) Appl. No.: 17/634,218

(22) PCT Filed: Aug. 12, 2020
(86) PCT No.: PCT/US2020/045962

§ 371 Date: Feb. 9, 2022

Related U.S. Application Data
(60) Provisional application No. 62/885,709, filed on Aug. 12, 2019.
(60) Provisional application No. 62/889,373, filed on Aug. 20, 2019.

(51) Int. Cl. G01N 33/574; C12Q 1/6886; G01N 33/68

(52) U.S. Cl. CPC G01N 33/57415; C12Q 1/6886; G01N 33/6848; C12Q 2600/158; C12Q 2600/156; G01N 2440/14

(57) ABSTRACT

Methods disclosed herein concern cancer proteogenomics, which integrates genomics, transcriptomics and mass spectrometry (MS)-based proteomics to gain insights into cancer biology and treatment efficacy. To promote clinical utility of embodiments herein, proteogenomics approaches were developed for frozen core needle biopsies using tissue-sparing specimen processing with or without a microscaled proteomics workflow.

This application claims priority to U.S. Provisional Patent Application Ser. No. 62/885,709, filed Aug. 12, 2019, and also claims priority to U.S. Provisional Patent Application Ser. No. 62/889,373, filed Aug. 20, 2019, both of which are incorporated by reference herein in their entirety.

### STATEMENT REGARDING FEDERALLY SPONSORED RESEARCH OR DEVELOPMENT

This invention was made with government support under CA214125, CA180860, and CA210986 awarded by the National Institutes of Health. The government has certain rights in the invention.

### TECHNICAL FIELD

Embodiments of the disclosure include at least the fields of molecular biology, cell biology, genetics, and medicine.

### BACKGROUND

Cancer proteogenomics integrates data from cancer genomics and transcriptomics with cancer proteomics to provide deeper insights into cancer biology and therapeutic vulnerabilities. Both by improving the functional annotation of genomic perturbations and by providing insights at the pathway level, this multi-dimensional approach to the characterization of human tumors has already shown considerable promise for the delineation of cancer biology and treatment options¹⁻⁴. In addition, proteogenomics applied to patient-derived xenograft (PDX) samples has exposed potential predictive markers and mechanisms of tumor response and resistance^{3,5,6}. Thus far, one limitation for proteogenomics is that the amount of tissue required has been fairly large, limiting translational research opportunities and applicability of mass spectrometry as an approach to cancer diagnostics. For example, the Clinical Proteomic Tumor Analysis Consortium (CPTAC) has required a minimum of 100 mg (wet weight) of tissue from a surgical resection specimen, which typically yields several hundred micrograms of protein and provides quantitative information on >10,000 proteins and >30,000 phosphosites per sample⁷. In these early projects, RNA, DNA and protein were often isolated from separate parts of the tumor and after variable sample ischemia periods of an hour or more, raising concerns related to sample heterogeneity and pre-analytical variability.

To make proteogenomics more applicable for clinical diagnostics, a “microscaled” approach could be employed, whereby, for example, a snap-frozen tumor-rich core needle biopsy (10 to 20 mg wet weight) may provide sufficient DNA, RNA and protein for deep-scale proteogenomic profiling that includes genome sequencing, RNA sequencing, and deep-scale mass spectrometry-based quantification of proteins and post-translational modifications. Effective microscaling would allow routine proteogenomic profiling of clinical biopsy specimens, including paired pre- and on-treatment analyses to facilitate assessment of on-target pathway inhibition and identification of compensatory resistance mechanisms. The examination of multiple cores could both illuminate intra-tumoral heterogeneity and help mitigate the challenges it presents. The present disclosure concerns methods and compositions to achieve these goals.

### BRIEF SUMMARY

The present disclosure concerns methods, systems, and compositions useful for treatment of an individual, including determining the treatment of an individual. In particular embodiments, multiple components from a biological sample are utilized to determine a treatment for an individual. In specific cases, the biological sample is from a biopsy, such as a biopsy from cancer tissue. The biopsy may comprise heterogeneous tissue and/or pluralities of cells. In some embodiments, compositions comprise sections from the biopsy, and in certain embodiments various sections from the biopsy are separated into distinct vessels with the purpose of the cells/tissues among the vessels having a uniform distribution of similar biological replicates.

In some embodiments, compositions comprise nucleic acids, such as DNA and/or RNA, and/or protein derived from the biopsy and/or sections of the biopsy. In some embodiments, the DNA, RNA, and/or protein are isolated into individual vessels, including distinct vessels comprising sections of the biopsy. The DNA, RNA, and/or protein isolated into individual vessels may or may not have originated from different regions of the biopsy, such that the isolated DNA, RNA, and/or protein comprises DNA, RNA, and/or protein originating from different regions of the biopsy. In some embodiments, the compositions further comprise preparations of the sections for microscopic analysis.

In certain embodiments, some sections of the biopsy are combined with other sections of the biopsy. The combining of some sections with other sections may comprise combining sections of one region of the biopsy with at least one other region of the biopsy. In certain embodiments, non-adjacent sections from the biopsy are combined, including non-adjacent sections from different regions of the biopsy.

In some embodiments, the isolated DNA, RNA, and/or protein is analyzed by any method known in the art. Analyzing DNA, RNA, and/or protein may comprise, for example, any PCR technique (such as qPCR, RT-qPCR, and/or digital PCR), the use of restriction enzymes (such as for restriction fragment length polymorphism analysis or the like), any sequencing technique (such as Sanger sequencing, next generation sequencing, high throughput sequencing, deep sequencing, nanopore sequencing, exome sequencing, and/or single cell sequencing), Northern blotting, Western blotting, Southern blotting, flow cytometry, mass spectrometry, NMR spectroscopy, electrophoresis, or a combination thereof. In particular embodiments, one or more proteins and/or one or more peptides are analyzed by mass spectrometry but the processes to analyze nucleic acid can include options of types of analyses.

In some embodiments, the DNA, RNA, and/or protein is analyzed to measure the status, such as the levels, presence, and/or absence, of certain one or more molecular markers. The molecular markers may be any marker, such as a biomarker, including molecular markers useful for the diagnosis or prognosis (or combination thereof) of cancer in an individual. In some embodiments, the molecular markers are one or more proteins and/or one or more peptides and/or one or more nucleic acids encoding proteins or peptides selected from the group consisting of: members of the ErbB receptor family (including but not limited to ERBB2 (also known as HER2 and HER2/neu), ERBB3, and ERBB4), Mucin-1, Mucin-6, PD-1, PD-L1, STAR3, GRB7, mTOR (or subunits of mTORC), members of interferon signaling components, AKT, SHC1, EIF4EBP1, and a combination thereof. In some embodiments, the status of certain one or more molecular markers is determined by determining the post-translational modification status (such as phosphorylation status) of the molecular marker, such as determining the presence or absence of a phosphate at one or more locations on the molecular marker. In some embodiments, the molecular markers comprise phosphorylation markers on proteins, including any protein encompassed by the present disclosure. Phosphorylation status may be determined by any method known in the art, including any mass spectrometry technique capable of detecting phosphorylation, for example.

Certain embodiments concern measuring the status of HER2 DNA, RNA, and/or protein. In some embodiments the phosphorylation status of HER2 is determined. The phosphorylation status of HER2 may be determined by mass spectrometry, including any mass spectrometry method encompassed herein.

Certain embodiments of the disclosure concern the analysis of one or more protein samples. The protein samples may be from any source, including from a biological sample of any kind, including a biopsy of any kind. In some embodiments, protein from a biological sample is digested into peptides. Any method known in the art for digesting proteins into peptides may be used, such as LysC, trypsin, and/or chemotrypsin digestion, as examples. In some embodiments, the peptides are tagged with one or more unique tags of known molecular weight. The peptides derived from each protein sample may get a unique tag specific to that protein, such that peptides are able to be identified based on the protein sample from which the peptides were derived. In some embodiments, after the peptides have been tagged, the tagged peptides are combined into at least one vessel. In some embodiments, the combined, tagged peptides may be subjected to sorting. Any method known in the art for sorting peptides may be used. Examples of methods for sorting peptides include chromatography, such as reverse-phase chromatography or basic reverse-phase chromatography, immunoprecipitation, affinity sorting, electrophoresis, or a combination thereof, for example. Peptides may be sorted by size, charge, polarity, solubility, isoelectric point, affinity to other molecules, presence or absence of at least one posttranslational modification, such as phosphorylation, acetylation, ubiquitylation, methylation, or a combination thereof. In some embodiments, the tagged peptides are subject to mass spectrometry analysis. In some embodiments, the tagged and sorted peptides are subject to mass spectrometry analysis.

Certain embodiments of the present disclosure concern employing methods of the present disclosure for diagnosing, prognosticating, and/or treating an individual having cancer or suspected of having cancer. Any method described herein may be used for diagnosing, prognosticating, and/or treating an individual having, or suspected of having, cancer. In some embodiments, the methods encompassed in the present disclosure are employed to treat an individual with a particular treatment, including a particular cancer treatment. As one example, the treatment may be a HER2-targeted treatment, including at least as part of a treatment regimen. In such embodiments, a biopsy from an individual having, or suspected of having, cancer may be subjected to any of the methods encompassed in the present disclosure to determine whether the individual has cancer cells positive for one or more particular markers, for example HER2. The individual determined to have cancer cells positive for the marker may be administered a therapeutically effective amount of a treatment. For example, an individual determined to have cancer cells positive for HER2 may be administered a therapeutically effective amount of at least one HER2-targeted treatment. In some embodiments, wherein the individual was determined not to have cancer cells positive for HER2 based upon utilizing methods encompassed herein, at least one non-HER2-targeting therapy may be used. A HER2-targeted treatment may be any composition that targets, inhibits, antagonizes, reduces, degrades, binds, or a combination thereof, HER2 DNA, HER2 RNA, and/or HER2 protein. A HER2-targeted treatment may be any composition that inhibits any post-translational modification, such as phosphorylation, on HER2 protein. An example of a targeted treatment is an antibody or antibody-related composition of any kind.

In some embodiments, methods encompassed in the present disclosure may be employed to determine whether an individual having, or suspected of having, cancer has cancer cells susceptible to one or more particular treatments, including one or more HER2-targeted treatments. In such embodiments, at least one additional biopsy taken from the individual at least 1, 2, 3, 4, 5, 6, 7, 8, 9, 10, or more days after the initial administration of a therapy, including the targeted therapy, is subjected to any of the methods encompassed herein. In some embodiments, a status of one or more molecular markers changes when comparing a biopsy taken from the individual prior to a treatment vs. following one or more treatments. In such cases, the individual may be administered one or more therapeutically effective amounts of at least one different treatment (including a targeted treatment) than had been utilized. In some embodiments, wherein the status of one or more molecular markers did not change in a biopsy taken from the individual relative to the first biopsy taken from the individual, the individual may be administered a therapeutically effective amount of one or more treatments of the original type of treatment.

Certain methods encompassed herein concern the detection of HER2 status in an individual, such as by subjecting a biological sample from the individual to any of the methods encompassed herein.

In some embodiments, any of the methods encompassed herein may be used to distinguish cells and/or tissue positive for one or more particular markers and/or cells and/or tissue negative for one or more particular markers. For example, HER2-positive cells and/or tissue may be distinguished from HER2-negative cells and/or tissue when utilizing methods encompassed herein.

It is specifically contemplated that any limitation discussed with respect to one embodiment of the disclosure may apply to any other embodiment of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Brief Summary, Detailed Description, Claims, and description of Brief Description of the Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure. Additional objects, features, aspects and advantages of the present invention will be set forth in part in the description which follows, and in part will be obvious from the description or may be learned by practice of the invention. Various embodiments of the disclosure will be described in sufficient detail to enable those skilled in the art to practice the invention, and it is to be understood that other embodiments may be utilized and that changes may be made without departing from the scope of the invention. The following detailed description is, therefore, not be taken in a limiting sense, and the scope of the present invention is best defined by the appended claims.

### BRIEF DESCRIPTION OF THE DRAWINGS

For a more complete understanding of the present disclosure, reference is now made to the following descriptions taken in conjunction with the accompanying drawings.

FIGS. 1A-1B describe an example workflow for at least one method encompassed herein, such as a biopsy-trifecta extraction (BioTExt) based proteogenomics workflow. FIG. 1A) Overviews an embodiment of the BiTExt protocol, wherein patient derived OCT-embedded needle core biopsies were sectioned, followed up by extraction of DNA, RNA and proteins for deep-scale proteogenomics characterization and by immunohistochemistry based imaging. FIG. 1B) Overviews an embodiment of the Microscaled proteomics (MiProt) workflow, which allows deep-scale proteomics and phosphoproteomics characterization with 25 μg of peptides per core-needle biopsy. MiProt may use a common reference that may or may not be used for comparison across all samples within a single-TMT10/11plex and across several TMT10/11plexes spanning several core biopsies.

FIGS. 2A-2F provide an evaluation of BioText and MiProt workflow on preclinical PDX models. FIG. 2A) Non-adjacent, core needle biopsies were collected from 2 basal and 2 luminal PDX models followed by surgical removal of tumors. Proteomic and phosphoproteomic characterization of cores were performed using MiProt workflow, and the bulk tissue was characterized using CPTAC workflow described in Mertins⁸. FIG. 2B) Venn-diagram showing the number and overlap between human and mouse or human proteins quantified in cores and bulk tissue. FIG. 2C) Venn-diagram shows the overlap between human and mouse or human phosphosites. FIG. 2D) Pearson correlation of TMT ratios for proteins (left) and phosphosites (right) between cores and bulk across all 4 PDX models. FIG. 2E) The heatmap shows the TMT ratios for key differentially regulated Luminal versus Basal breast cancer associated proteins and phosphoproteins (average expression of identified phosphosites) identified across both bulk and cores experiments. FIG. 2F) Gene-centric and phosphosite-centric pathway or kinase activity enrichment analysis was performed using GSEA (MSigDB “Cancer Hallmarks”, left) and PTM-SEA (PTMSigDB, right), respectively, for Luminal-Basal differences captured in bulk (y-axis) and core (x-axis) tissue. limma derived signed Log 10 p-values were used to pre-rank differential features for both GSEA and PTM-SEA analysis. The pathway/phospho-signatures that are significant in both cores and bulk are indicated in brown.

FIGS. 3A-3E show an application of microscaled proteogenomics to the “Discovery protocol 1” clinical trial (DP1), a small scale trial focused on Trastuzumab-based neoadjuvant chemotherapy. FIG. 3A) Overview of proteogenomics samples obtained from pre- and on-treatment core biopsies from the DP1 clinical trial. Each block indicates the data obtained from a separate core. FIG. 3B) Microscaled proteogenomics achieves a high level of proteogenomics depth for the DP1 core needle biopsies. Table summarizing total proteogenomics coverage and numbers of mutated genes for all samples and average coverage across all analyzed cores is shown on the left. The Venn-diagram on the right shows the overlap between all genes identified across RNA-seq, proteomics and phosphoproteomics. FIG. 3C) Heatmap summarizing genomic alterations of breast cancer associated genes in tumors from 14 patients. FIG. 3D) Heatmap summarizing proteogenomics features of ERBB2 amplicon and adjacent genes at the level of copy number analysis (CAN), RNA and Protein expression. The set of genes in red make up the core of the ERBB2 amplicon and showed consistently high copy number amplification, RNA, and protein levels in all of the pathological Complete Response (pCR) cases but significantly lower protein levels in BCN1326, BCN1331, and BCN1335. The arrow points to TOP2A amplification. FIG. 3E) The heatmap at the bottom shows corresponding Z-scores of RNA, protein, and phosphoprotein expression of ERBB1-4 across all 14 patients. ERBB3 protein and phosphoprotein and ERBB4 protein levels were also significantly lower in BCN1326, BCN1331, and BCN1335 than in the set of pCR cases.

FIGS. 4A-4C show that dual anti-ERBB2 therapy results in downregulation of ERBB2 and mTOR signaling in cases with pCR. FIG. 4A) Effect of anti-ERBB2 treatment on ERBB2 RNA, protein, and phosphoprotein levels for each patient with on-treatment data. P-values for paired Wilcoxon signed rank tests for on-treatment vs. pre-treatment ERBB2 expression for each group. The pCR vs. non-pCR p-values are derived from Wilcoxon rank sum tests comparing log 2 fold changes of on-treatment to pre-treatment levels from pCR patients to those from non-pCR patients. For patients with multiple cores, the mean expression value was used. FIG. 4B) Scatter plot showing differential regulation of phosphosites before and after treatment in pCR and in non-pCR cases. Shown are the on-treatment vs. pre-treatment log 2 fold changes in non-pCR (y-axis) vs. the log 2 changes in pCR samples (x-axis) for phosphosites with p-value <0.05 by limma analysis of differential expression in either group. Blue and green circles indicate phosphosites in pCR and non-pCR, respectively that show significant differential regulation in either group alone. Purple circles indicate significantly regulated phosphosites in both sets of patients. The orange diamond outlines highlight phosphosites on proteins in the KEGG ErbB signaling pathway (hsa04012). The transparency of each point reflects its significance after BH-adjustment (adjusted p<0.05 is solid, and more transparent points have higher adjusted p-values). FIG. 4C) PTM-SEA was applied to the signed Log 10 p-values from limma differential expression analysis of on- vs. pre-treatment phosphosite levels from pCR cases (upper panel) and non-pCR (lower panel). The volcano plot shows the Normalized Enrichment Scores (NES) for kinase signatures. Red circles indicate signatures with significant FDR (<0.05).

FIGS. 5A-5F show that proteogenomics analysis of baseline untreated samples suggest diverse “candidate” mechanisms of resistance in non-pCR cases. FIG. 5A) Outlier analysis was performed to identify differentially regulated mRNA, proteins or phosphoproteins in each pre-treatment sample from non-pCR cases relative to the set of pre-treatment samples from all pre-treated pCR cases. Shown is the ERBB2 protein distribution across all patients; brown and green bars indicate the frequencies for each protein level bin in non-pCR and pCR, respectively. The line shows the normal distribution of pCR samples from which the Z-score for each non-pCR sample was derived. Corresponding Z-scores levels are indicated in red. FIG. 5B) Heatmap showing normalized enrichment scores (NES) from single sample Gene Set Enrichment Analysis (ssGSEA) of outlier Z-scores from non-pCR cases. Shown are a subset of differentially regulated pathways with false-discovery rate less than 25% (FDR <0.25). FIG. 5C) Heatmap showing expression levels of key immune-checkpoint and T-cell marker (CD3) genes and of RNA based immune and stroma scores from ESTIMATE, Cibersort, and xCell. FIG. 5D) Photomicrographs showing anti-CD3 immunohistochemical staining profiles of non-pCR cases (original magnification: 20×). FIG. 5E) Heatmap showing Mucin protein expression across all pre-treated patients. FIG. 5F) WHIM8 and WHIM35 PDX models were treated with vehicle, trastuzumab, everolimus or the combination of trastuzumab and everolimus. The graph shows the mean-tumor volume at several timepoints after tumor implantation and subsequent treatment, and error bars show standard error of mean.

FIGS. 6A-6B show DNA, RNA and protein yields from core needle biopsy processed using BioTExt. FIG. 6A) Box plot showing DNA, RNA and Protein yields from a total of 8 core needle biopsies from 4 PDX Models: WHIM4, 14, 18 and 20. FIG. 6B) Box and scatter plots showing DNA, RNA and protein yields from all core needle biopsies that were processed. Samples with no yield were excluded. Error bars represent standard error of mean (SEM).

FIGS. 7A-7D show comparison of proteomics and phosphoproteomics dataset from tumor bulk and core samples. FIG. 7A) The table shows the number of proteins and phosphosites quantified in the bulk tissue (upper panel) and non-adjacent (lower panel) cores from 4 WHIM PDX models. FIG. 7B) The table lists the Pearson correlation between replicate bulk and non-adjacent cores for each of the PDX models FIG. 7C) Unsupervised hierarchical clustering (1-Pearson) of normalized TMT protein and phosphosite ratios. FIG. 7D) ssGSEA was performed on normalized TMT protein ratios obtained from cores and bulk. Scatter plot shows ssGSEA normalized enrichment scores (NES) between cores and bulk tissue for individual PDX models.

FIG. 8 shows the Reporting Recommendations for Tumor Marker Prognostic Studies (REMARK) diagram. The flowchart shows the number of patients enrolled in the trial and reasons for their exclusion from the proteogenomic analysis when applicable.

FIGS. 9A-9C show proteogenomics features of the clinical cores. FIG. 9A) The right panel shows scatter plot between percentage of target base at 50× sequencing depth and mean bait coverage for whole-exome sequencing and the left panel shows comparable box plot distribution of percentage of target based at 50× sequencing depth for DNA isolated from blood versus tumor using BioTExt. FIG. 9B) The copy number landscape of ERBB2+ samples from TCGA (top) resembles the landscape from this study (bottom). Plots show log 2 ratios of chromosome segment copy number in tumor DNA relative to normal DNA for each patient (rows) from each cohort. FIG. 9C) Distribution of gene-wise Spearman correlations between RNA and Protein as observed using the BioTExt pipeline. Red and green indicate all positively and negatively correlated genes respectively.

FIGS. 10A-10B show functional prediction from co-expression networks derived from and unsupervised hierarchical clustering of samples from clinical core proteogenomics data. FIG. 10A) Co-expression networks derived from microscaled proteomics data predict function more consistently than co-expression networks derived from the RNA data. Red and blue circles indicate functional categories (KEGG pathways) predicted by co-expression networks derived from protein and mRNA expression data, respectively. FIG. 10B) Core needle biopsies from the same patients cluster together based on the top 500 most variable features in each dataset.

FIGS. 11A-11D show validation of ERBB2 levels. FIG. 11A) ERBB2 (HER2) immunohistochemistry (IHC) on sections from all 14 patients, wherein photomicrographs showing ERBB2 IHC staining profiles of all pCR cases at 200×. FIG. 11B) Box plot showing ERBB2 IHC scores and ERBB2 protein levels. FIG. 11C) Box plot showing ERBB2 IHC scores and ERBB2 protein levels as measured by parallel reaction monitoring (PRM). FIG. 11D) Scatter plot showing correlation between ERBB2 protein abundance measured using TMT and PRM based protein quantification.

FIGS. 12A-12C show scatter plots of response to treatment (on-treatment vs pre-treatment) in non-pCR (y-axis) vs. pCR patients for RNA (FIG. 12A), proteins (FIG. 12B), and (FIG. 12C) phosphoproteins (mean of phosphosites). Shown are log 2 ratios from limma linear modeling of differential expression for genes with p<0.05 in each set of patients. Genes from the ERBB signaling KEGG pathway (hsa04012) are highlighted in orange. The level of transparency of each point reflects its significance after BH-adjustment (adjusted p<0.05 points are completely opaque, and more transparent points have higher adjusted p-values).

FIG. 13 shows PTM-SEA analysis on pre and on-treatment phosphoproteomics dataset. PTM-SEA was applied to the signed Log 10 p-values from limma differential expression analysis of on- vs. pre-treatment phosphosite levels from pCR cases (orange) and non-pCR (green) The heatmap shows the Normalized Enrichment Scores (NES) for these kinase signatures, and asterisks indicate significant FDR (<0.05).

FIG. 14 shows outlier analysis was performed to identify differentially regulated mRNA, proteins or phosphoproteins in each pre-treatment sample from non-pCR cases relative to the set of pre-treatment samples from all pre-treated pCR cases. Shown is the ERBB2 (HER2) RNA and phosphoprotein distribution across all patients; brown and green bars indicate the frequencies for each protein level bin in non-pCR and pCR, respectively. The line shows the normal distribution of pCR samples from which the Z-score for each non-pCR sample was derived. Z-score thresholds are indicated by red lines.

FIG. 15 shows expression of key immune checkpoint regulators in and immunoprofiling of pre-treated samples. Upper panel shows Z-scores of RNA, protein, and phosphoprotein expression (where available) of key immune checkpoint inhibitors in each baseline sample from pCR (samples on right) and non-pCR (samples on left) patients. Bottom panel shows Z-scores of immune cell profiles inferred from RNA-seq data using Cibersort.

FIGS. 16A-16B show ERBB2 (HER2) immunohistochemistry of patient samples. FIG. 16A) Hematoxylin and eosin (HE) staining of patient samples. HE staining of tissue sections from all 14 patients. The middle panel showed magnified HE stains (400×) of sections patients AR+ patients 1331 and 1371. Patient 1371 shows distinct apocrine features as indicated by plump pink cytoplasm. FIG. 16B) Immunohistochemical staining profiles of non-pCR cases for AR at 200×

FIG. 17 shows association of outliers with publications with keywords “breast cancer” and “resistance” or “recur”. Z-score for each gene from outlier analysis is plotted on the y-axis, while the x-axis indicates the number of publications associated with that gene and with breast cancer resistance terms. A separate plot is included for outliers for each non-pCR sample from each omics dataset.

FIGS. 18A-18B show ERBB2 and MUCIN expression in WHIM35, WHIM8 and patient BCN1326. FIG. 18A) Heatmap showing ERBB2 pathway and Mucin protein expression in two HER2-enriched PDX (WHIM) models with ERBB2 protein expression. FIG. 18B) MUC1 immunohistochemistry (IHC) of WHIM8, WHIM35 and BCN1369.

FIGS. 19A-19B show proteogenomic classification of HER2 status in breast cancer patients. FIG. 19A) Proteogenomic (PG) analysis of the ERBB2 locus in three breast cancer cohorts: prospectively collected BRCA tumors; biopsies from ERBB2 positive BRCA tumors (Discovery Protocol (DP) 1; (Satpathy et al., Nature Communications volume 11, Article number: 532 (2020)); and retrospective analysis of TCGA tumors (data from Mertins et al., Nature. 2016 May 25; 534(7605): 55-62). The heatmap depicts clinical data (upper panel), copy number alterations (middle panel) and protein expression (lower panel) of genes proximal to ERBB2 on chromosome 17q for samples from each study. PG amplification of TOP2A, a potential alternative driver in the locus, in psuedo-ERBB2+ tumors is indicated by red arrowheads and blue boxes. FIG. 19B) Outlier analysis confirms higher protein levels in most ERBB2 amplified samples (purple histogram) relative to the distribution for ERBB2 protein in non-amplified samples (blue histogram) in the prospective and retrospective datasets. Amplified samples with Z-scores >2 for ERBB2 and STARD3 or GRB7 proteins are considered PG positive whereas samples with protein levels falling within the distribution of ERBB2 non-amplified samples are considered “pseudo ERBB2 positive”.

### DETAILED DESCRIPTION

This application incorporates by reference herein in its entirety U.S. Provisional Patent Application Ser. No. 62/885,709, filed Aug. 12, 2019.

### I. Examples of Definitions

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “comprising,” which is synonymous with “including,” “containing,” or “characterized by,” is inclusive or open-ended and does not exclude additional, unrecited elements or method steps. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments described in the context of the term “comprising” may also be implemented in the context of the term “consisting of” or “consisting essentially of.”

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

The term “sample,” as used herein, generally refers to a biological sample. The sample may be taken from tissue and/or cells and/or from the environment of tissue or cells. In specific cases the cells and/or tissues are cancer cells or suspected cancer cells, including from a tumor or suspected tumor or tumor microenvironment or suspected tumor microenvironment. In some examples, the sample may comprise, or be derived from, a tissue biopsy, blood (e.g., whole blood), blood plasma, extracellular fluid, dried blood spots, cultured cells, culture media, discarded tissue, or a combination thereof. The sample may have been isolated from the source prior to collection. The sample may be fresh or frozen prior to analysis. Non-limiting examples include blood, cerebral spinal fluid, pleural fluid, amniotic fluid, lymph fluid, saliva, urine, stool, tears, sweat, or mucosal excretions, and other bodily fluids, including isolated from the primary source prior to collection. In some examples, the sample is isolated from its primary source (cells, tissue, bodily fluids such as blood, environmental samples, etc.) during sample preparation. The sample may or may not be purified or otherwise enriched from its primary source. In some cases the primary source is homogenized prior to further processing. The sample may be filtered or centrifuged to remove buffy coat, lipids, or particulate matter. The sample may also be purified or enriched for nucleic acids, or may or may not be treated with RNases. The sample may contain tissues or cells that are intact, fragmented, or partially degraded. The sample may be separated for further analysis, including into different vessels for analysis.

The term “subject,” as used herein, generally refers to an individual having a biological sample that is undergoing processing or analysis and, in specific cases, has cancer or is suspected of having cancer. The subject can be any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject can be a patient, e.g., have or be suspected of having a disease (that may be referred to as a medical condition), such as one or more one or more cancers, or any combination thereof. The subject may being undergoing or having undergone treatment. The subject may be asymptomatic. The subject may be in need of cancer treatment. The term “individual” may be used interchangeably, in at least some cases. The “subject” or “individual”, as used herein, may or may not be housed in a medical facility and may be treated as an outpatient of a medical facility. The individual may be receiving one or more medical compositions via the internet. An individual may comprise any age of a human or non-human animal and therefore includes both adult and juveniles (i.e., children) and infants and includes in utero individuals. It is not intended that the term connote a need for medical treatment, therefore, an individual may voluntarily or involuntarily be part of experimentation whether clinical or in support of basic science studies. The subject may be healthy.

The present disclosure concerns methods that facilitate analysis of biological samples for accurate treatment or prognosis for an individual. In particular cases, an individual that has cancer or is suspected of having cancer is subject to methods of the disclosure to provide a correct assessment to allow for selection of one or more suitable treatments. The methods greatly reduce the risk of inaccurate treatment regimens at least in part because they employ proteogenomics that encompass analysis of DNA, RNA, and protein as part of the evaluation for the individual. The particular methods of the disclosure, however, reduce the level of required tissue and include uniform distribution of sample parts (such as sections) each for the DNA analysis, RNA analysis, and protein analysis. The DNA analysis, RNA analysis, and protein analysis may or may not occur in parallel, although in particular cases the different analyses occur generally concomitantly. In specific cases, following distribution of the sample sections for the DNA analysis, RNA analysis, and protein analysis, mass spectrometry may be utilized to analyze the proteome and/or phosphoproteome from the biological sample. In specific cases, analysis of the proteome and/or phosphoproteome utilizes a scale of tissue on the order of micrograms.

### II. Biological Samples

Certain embodiments of the disclosure concern at least one biological sample of any kind, such as a biopsy of any kind, taken from an individual, including any individual encompassed herein. The individual may have cancer, may be suspected of having cancer, or may be at increased risk for having cancer compared to the general population (for example, a personal or family history, a smoker, the elderly, exposure to the sun or environmental conditions, a combination thereof, and so forth). The individual may be a research subject, including any mammal that is part of a research study. A biopsy may be obtained as part of a routine preventative practice or as part of a directed concern or suspected indication for the onset of cancer.

The one or more biological samples may be taken from the individual at any time, such as before, after, and/or simultaneously with diagnosis of a cancer, or such as before, after, and/or simultaneously with the administration of one or more therapies. At least one of the biological samples taken from the individual may be taken from a tumor or other cancer cells present in the individual. A tumor may or may not be benign or suspected of being benign. In some embodiments, at least one of the biological samples taken from the individual may be taken from non-cancerous tissue or other biological material in the individual. The biological sample may be taken from the individual using any method known in the art, including a(n) needle biopsy (including core-needle biopsy), guided biopsy, aspiration biopsy, surgical biopsy, core biopsy, open biopsy, punch biopsy, sentinel lymph node biopsy, shave biopsy, endoscopic biopsy, or a combination thereof. In some embodiments, the biological sample is taken from the individual by a core-needle biopsy, including a core-needle biopsy using a 14 gauge, 15 gauge, 16 gauge, 17 gauge, 18 gauge, 19 gauge, 20 gauge, 21 gauge, or 22 gauge needle. The biological sample may be from tissue, bone, blood, serum, plasma, urine, stool, sputum, saliva, semen, vaginal fluids, mucus, fat, or a combination thereof. In specific embodiments, a biological sample comprises a biopsy that is taken from a mass in a breast of an individual.

The biological sample may be prepared, processed, stored, handled, and/or fixed using any method known in the art. The sample may or may not be stored prior to processing. In some embodiments, at least one biological sample is embedded in optimal cutting temperature (OCT) medium. In some embodiments, at least one biological sample is stored in cryogenic storage, such as at a temperature lower than −80° C., lower than −70° C., lower than −60° C., lower than −50° C., lower than −40° C., lower than −30° C., or lower than −20° C. In some embodiments, at least one biological sample is prepared and/or sectioned using a microtome, such as a crytostat. In some embodiments, the sectioning is done at a temperature lower than −30° C., lower than −20° C., or lower than −10° C. In some embodiments, the sectioning is done at a temperature between −30° C. to −10° C., or between −23° C. to −15° C. In some embodiments, the biological sample is sectioned at a thickness between 3 microns and 100 microns, or between 4 microns and 100 microns, or between 5 microns and 100 microns, or between 3 microns and 50 microns, or between 4 microns and 50 microns, or between 5 microns and 50 microns. However, the biological sample may be sectioned to any thickness that is suitable for practicing the methods of the disclosure. In some embodiments, one or more sections from the biological samples are placed into one or more vessels suitable for comprising the sections.

### III. Production of DNA, RNA, and/or Protein

Certain embodiments of the present disclosure concern methods of generating DNA, RNA, and/or protein from at least one biological sample, such as a biopsy. In some embodiments, the DNA, RNA, and/or protein are isolated in individual vessels, including vessels comprising one or more sections of the biological sample(s). The DNA, RNA, and/or protein isolated into individual vessels may have originated from different regions of the biological sample, such that the isolated DNA, RNA, and/or protein in an individual vessel comprises DNA, RNA, and/or protein originating from different regions of the biological sample. In some embodiments, at least one biological sample contained in a vessel is used for the preparation of the section(s) for microscopic analysis.

In certain embodiments, some sections of the biological sample are combined with other sections of the biological sample. The combining of some sections with other sections may comprise combining sections of one region of the biological sample with at least one other region of the biological sample. In certain embodiments, non-adjacent sections from the biological sample are combined, including non-adjacent sections from different regions of the biological sample.

In some embodiments, at least three sections are generated from a biopsy, followed by adding any one of the three sections to a first vessel, adding any one of the two remaining sections to a second vessel, and adding the remaining section to a third vessel. The process may be repeated indefinitely. The processes may be repeated until a sufficient number of sections, from the biological samples, for practice of the disclosure are generated and placed into vessels. A sufficient number may be the number required to produce sufficient RNA, DNA, and/or protein for analysis. In some embodiments, four sections are generated followed by, in any order: adding any one of the four sections to a first vessel, adding any one of the four sections not in the first vessel to a second vessel, adding any one of the four sections not in the first or second vessel to a third vessel, and preparing any one of the four sections not in the first, second, or third vessel for microscopic analysis. The process may be repeated indefinitely. The processes may be repeated until a sufficient number of sections, from the biological samples, for practice of the disclosure are generated and placed into vessels and/or prepared for microscopic analysis. A sufficient number may be the number required to produce sufficient RNA, DNA, and/or protein for analysis.

In some embodiments, a sufficient number of sections for practice of the disclosure will produce approximately between 10 μg to 45 μg of isolated protein. In some embodiments, a sufficient number of sections for practice of the disclosure will produce approximately 10 μg, 15 μg, 20 μg, 25 μg, 30 μg, 35 μg, 40 μg, 45 μg, or more than 45 μg of isolated protein. In some embodiments, a sufficient number of sections for practice of the disclosure will produce approximately between 0.1 μg to 1 μg of isolated DNA. In some embodiments, a sufficient number of sections for practice of the disclosure will produce approximately 0.1 μg, 0.2 μg, 0.3 μg, 0.4 μg, 0.5 μg, 0.6 μg, 0.7 μg, 0.8 μg, 0.9 μg, 1.0 μg, or more than 1.0 μg of isolated DNA. In some embodiments, a sufficient number of sections for practice of the disclosure will produce approximately between 0.1 μg to 1 μg of isolated RNA. In some embodiments, a sufficient number of sections for practice of the disclosure will produce approximately 0.1 μg, 0.2 μg, 0.3 μg, 0.4 μg, 0.5 μg, 0.6 μg, 0.7 μg, 0.8 μg, 0.9 μg, 1.0 μg, or more than 1.0 μg of isolated RNA.

Certain embodiments of the disclosure concern the isolation of DNA, RNA, and/or protein from one or more sections generated from at least one biological sample. The DNA, RNA, and/or protein may be isolated using any method known in the art. In some embodiments, the DNA may be isolated from one or more sections of at least one biological sample by digesting the section(s) with a proteinase and an RNase, then purifying the DNA, such as by ethanol precipitation and/or a column purification system. In some embodiments, the RNA may be isolated from one or more sections of at least one biological sample by incubating the section(s) with an RNA extraction reagent, such as TRIzol reagent. The TRIzol reagent incubated sections may be sonicated and the organic layer may be extracted using an organic solvent, such as chloroform. The resulting RNA may be dissolved in a suitable solution (including water) and further purified, such as by ethanol precipitation and/or a column purification system. In some embodiments, protein, including native and/or denatured protein, may be isolated from one or more sections of at least one biological sample such as by, optionally precipitating the sections with ethanol, followed by incubation with a suitable lysis buffer. In some embodiments, the DNA, RNA, and/or protein isolated are subjected to quality control analysis.

### IV. Biological Sample Analysis of DNA, RNA, and/or Protein

Certain embodiments of the present disclosure concern methods for analyzing biological samples taken from an individual, including any individual encompassed herein. In some embodiments, DNA, RNA, and/or protein isolated from one or more sections of at least one biological sample is analyzed. Analyzing DNA, RNA, and/or protein may comprise, for example, any PCR technique (such as qPCR, RT-qPCR, and/or digital PCR), the use of restriction enzymes (such as for restriction fragment length polymorphism analysis or the like), any sequencing technique (such as Sanger sequencing, next generation sequencing, high throughput sequencing, deep sequencing, nanopore sequencing, exome sequencing, and/or single cell sequencing), Northern blotting, Western blotting, Southern blotting, flow cytometry, mass spectrometry, NMR spectroscopy, electrophoresis, or a combination thereof.

In some embodiments, DNA may be analyzed by sequencing. The DNA may be prepared for sequencing by any method known in the art, such as library preparation, hybrid capture, sample quality control, product-utilized ligation-based library preparation, or a combination thereof. The DNA may be prepared for any sequencing technique, including whole exome sequencing. In some embodiments, a unique genetic readout for each sample may be generated by genotyping one or more highly polymorphic SNPs. In some embodiments, sequencing, such as 76 base pair, paired-end sequencing, may be performed to cover approximately 70%, 75%, 80%, 85%, 90%, 95%, 99%, or greater percentage of targets at more than 20×, 25×, 30×, 35×, 40×, 45×, 50×, or greater than 50× coverage. In certain embodiments, mutations, SNPS, INDELS, copy number alterations (somatic and/or germline), or other genetic differences may be identified from the sequencing, such as whole exome sequencing, using at least one bioinformatics tool, including VarScan2, any R package (including CopywriteR) and/or Annovar.

In some embodiments, RNA may be analyzed by sequencing. The RNA may be prepared for sequencing by any method known in the art, such as poly-A selection, cDNA synthesis, stranded or nonstranded library preparation, or a combination thereof. The RNA may be prepared for any type of RNA sequencing technique, including stranded specific RNA sequencing. In some embodiments, sequencing may be performed to generate approximately 10M, 15M, 20M, 25M, 30M, 35M, 40M or more reads, including paired reads. The sequencing may be performed at a read length of approximately 50 bp, 55 bp, 60 bp, 65 bp, 70 bp, 75 bp, 80 bp, 85 bp, 90 bp, 95 bp, 100 bp, 105 bp, 110 bp, or longer. In some embodiments, raw sequencing data may be converted to estimated read counts (RSEM), fragments per kilobase of transcript per million mapped reads (FPKM), and/or reads per kilobase of transcript per million mapped reads (RPKM). In some embodiments, one or more bioinformatics tools may be used to infer stroma content, immune infiltration, and/or tumor immune cell profiles, such as by using upper quartile normalized RSEM data.

In particular embodiments, protein from samples is analyzed, including denatured protein. The protein may be analyzed by mass spectrometry. The protein may be prepared for mass spectrometry using any method known in the art. In specific embodiments, and regardless of the methods of analyzing protein, the protein is digested to produce peptides that are then analyzed. Protein, including any isolated protein encompassed herein, may be treated with DTT followed by iodoacetamide. The protein may be incubated with at least one peptidase, including an endopeptidase, proteinase, protease, or any enzyme that cleaves proteins. In some embodiments, protein is incubated with the endopeptidase, LysC and/or trypsin. The protein may be incubated with one or more protein-cleaving enzymes at any ratio, including a ratio of μg of enzyme to μg protein at approximately 1:1000, 1:100, 1:90, 1:80, 1:70, 1:60, 1:50, 1:40, 1:30, 1:20, 1:10, 1:1, or any range between. In some embodiments, the cleaved proteins may be purified, such as by column purification. In certain embodiments, purified peptides may be snap-frozen and/or dried, such as dried under vacuum. In some embodiments, the purified peptides may be fractionated, such as by reverse phase chromatography or basic reverse phase chromatography. Fractions may be combined for practice of the methods of the disclosure. In some embodiments, one or more fractions, including the combined fractions, are subject to enrichment based on one or more post-translational modifications, such as phosphopeptide enrichment, including phospho-enrichment by affinity chromatography and/or binding, ion exchange chromatography, chemical derivatization, immunoprecipitation, co-precipitation, or a combination thereof. The entirety or a portion of one or more fractions, including the combined fractions and/or phospho-enriched fractions, may be subject to mass spectrometry. In some embodiments, the raw mass spectrometry data may be processed and normalized using at least one relevant bioinformatics tool. In specific cases, the protein is analyzed with mass spectrometry instead of antibody-based analysis.

In one embodiment, one can carry out further enrichments from the unbound fraction from the phospho-enrichment step to obtain e.g., acetylated peptide or other modified peptides.

### V. Applications of Embodiments of the Methods

Certain embodiments of the present disclosure concern employing methods of the present disclosure, such as the analysis of DNA, RNA, and/or protein, for diagnosing, prognosticating, and/or treating an individual having, or suspected of having, cancer. Any method described or encompassed herein may be used for diagnosing, prognosticating, and/or treating an individual having, or suspected of having, cancer. In some embodiments, the methods encompassed in the present disclosure are employed to treat an individual with a particular targeted treatment. The methods of the disclosure allow for accurate detection of cancer cells positive for one or more particular markers, following which a particular treatment is then employed based on the detection.

Merely as an illustrative case for breast cancer, for example, the methods of the disclosure allow for accurate assessment for breast cancer treatment, such as HER2-targeted treatment, for example. Any strategy herein referring to HER2 can apply to any cancer marker other than HER2. In such embodiments, a biopsy from an individual having, or suspected of having, cancer may be subjected to any of the methods encompassed in the present disclosure to determine whether the individual has cancer cells positive for HER2. The individual determined to have cancer cells positive for HER2 may be administered a therapeutically effective amount of at least one HER2-targeted treatment. In some embodiments, wherein the individual was determined not to have cancer cells positive for HER2, at least one non-HER2-targeting therapy may be used.

In some embodiments, methods encompassed in the present disclosure may be employed to determine whether an individual having, or suspected of having, cancer has cancer cells susceptible to one or more HER2-targeted treatments. As such, methods of monitoring a therapy are encompassed herein. In such embodiments, at least one biopsy is taken from the individual prior to or simultaneously with the administration of one or more HER2-targeted treatments and at least one additional biopsy is taken from the individual at least 1, 2, 3, 4, 5, 6, 7, 8, 9, 10, or more days after the initial administration of the one or more HER2-targeted therapies. A pre-therapy biopsy for analysis may be compared to analysis results from a post-therapy biopsy. In some cases, the post-therapy biopsy may be taken after the first dose, second dose, third dose, fourth dose, any subsequent dose, and so on. The biopsies are subjected to any method encompassed herein. In some embodiments, wherein the status of one or more molecular markers changed when comparing a biopsy taken from the individual after the initial administration of the one or more therapies relative to the status of the molecular marker(s) in a biopsy taken from the individual prior to or simultaneously with the administration of the therapies, the individual may be administered one or more additional therapeutically effective amounts of the same treatment. In some embodiments, wherein the status of one or more molecular markers did not change when comparing a biopsy taken from the individual after an initial administration of one or more therapies relative to the status of the molecular marker(s) in a biopsy taken from the individual prior to or simultaneously with the administration of the one or more therapies, the individual may be administered a different therapy.

Certain methods encompassed herein concern the detection of marker status in an individual, such as by subjecting a biological sample from the individual to any of the methods encompassed herein. In some embodiments, any of the methods encompassed herein may be used to distinguish certain marker-positive cells and/or tissue from certain marker-negative cells and/or tissue.

In specific embodiments, an individual that is known to have or is suspected of having HER2-positive cancer is provided a HER2-targeted treatment, and the efficacy of the treatment may be monitored. As used herein, a “HER2-targeted treatment” may describe any composition that targets, inhibits, antagonizes, reduces, degrades, binds, or a combination thereof, HER2 DNA, HER2 RNA, and/or HER2 protein. In some embodiments, a HER2-targeted treatment may be any composition that inhibits post-translational modifications, such as phosphorylation, on HER2 protein. HER2-targeted treatments include, but are not limited to, trastuzumab (Herceptin), pertuzumab (Perjeta), ado-trastuzumab emtansine (Kadcyla), lapatinib, neratinib (Nerlynx), any RNAi molecule targeting a nucleic acid that encodes HER2, or a combination thereof.

In some embodiments, at least one treatment other than a HER2-targeted treatment may be used or administered, which may comprise at least one chemotherapy, at least one immunotherapy, at least one biological therapy, at least one targeted therapy, at least one hormone therapy, or a combination thereof. In some embodiments, the treatment may comprise at least one checkpoint inhibitor, such as a PD-(L) 1 inhibitor, including nivolumab, pembrolizumab, atezolizumab, avelumab, durvalumab, cemiplimab; a CTLA4 inhibitor, including ipilimumab; or a combination thereof. In some embodiments, the treatment may comprise at least one anthracycline, such as daunorubicin, doxorubicin, epirubicin, idarubicin, or a combination thereof. In some embodiments, the treatment may comprise at least one mTOR inhibitor, such as rapamycin, everolimus, temsirolimus, sirolimus, ridaforolimus, or a combination thereof.

To determine whether or not an individual needs to have a particular treatment, one can examine the ranges for one or more proteins (for example HER2) in patients with a pathological Complete Response (pCR) to determine whether or not they receive a particular treatment. This may or may not be combined with conventional testing criteria. As one example, one can determine whether a case is a true false positive, i.e., central analysis using standard HER2 criteria agrees with the proteogenomics that the case is negative. In another case, one can be determined to be a pseudo positive, i.e the case is positive by conventional criteria but negative by proteogenomic criteria (the Top2a example herein) or there may be a true positive (both conventional criteria and proteogenomics agree) but the individual is resistant, i.e., no pCR (the Mucin example herein) or a true positive (both agree and patient experiences pCR).

### VI. Examples of Cancer Markers

Certain embodiments of the disclosure concern the analysis of DNA, RNA, and/or protein (but particularly all three) to measure the status, such as the levels, presence, and/or absence, of one or more certain molecular markers. A marker may be a protein, peptide, and/or mutated and/or post-translationally modified versions thereof. The cancer marker(s) (which also may be referred to herein as molecular markers) may be any marker, such as a biomarker, including molecular markers useful for the diagnosis or prognosis of cancer in an individual. In some embodiments, the one or more markers are characterized as markers for cancer because they are expressed on the surface of cancer cells, and their presence on the surface dictates a suitable therapy to target the marker-positive cancer cells. In specific cases, the molecular markers are proteins and/or one or more nucleic acids encoding proteins selected from the group consisting of: members of the ErbB receptor family (including but not limited to ERBB2 (also known as HER2 and HER2/neu), ERBB3, and ERBB4), Mucin-1, Mucin-6, PD-1, PD-L1, STAR3, GRB7, mTOR (or subunits of mTORC), members of interferon signaling components, AKT, SHC1, EIF4EBP1, TOP2A, and a combination thereof. In some embodiments, the status of certain one or more molecular markers is determined by determining the post-translational status (such as phosphorylation status) of the molecular marker, such as determining the presence or absence of a phosphate at one or more particular locations on the molecular marker. In some embodiments, the molecular markers are phosphorylation markers on proteins, including any protein encompassed by the present disclosure. Phosphorylation status may be determined by any method known in the art, including any mass spectrometry technique capable of detecting phosphorylation, for example.

Particular embodiments of the disclosure concern measuring the status of HER2 DNA, RNA, and/or protein, and such a status provides information about a diagnosis, prognosis, and/or treatment for the individual. In some embodiments the phosphorylation status of HER2 is determined. The phosphorylation status of HER2 may or may not be determined by mass spectrometry.

Certain embodiments of the disclosure concern measuring the status of one or more peptides. The peptides may comprise any peptide derived from any protein encompassed herein, including those described in the figures encompassed herein. In some embodiments, the peptides comprise any peptide derived from a protein selected from the group consisting of: members of the ErbB receptor family (including but not limited to ERBB2 (also known as HER2 and HER2/neu), ERBB3, and ERBB4), Mucin-1, Mucin-6, PD-1, PD-L1, STAR3, GRB7, mTOR (or subunits of mTORC), members of interferon signaling components, AKT, SHC1, EIF4EBP1, TOP2A, and a combination thereof. In some embodiments, the phosphorylation status, such as the presence or absence of a phosphate group on one or more particular residues, of the peptide(s) is measured. In some embodiments, the molecular markers are represented by the peptides in Table 1. In some embodiments, the phosphorylation status of one or more residues of one or more peptides of Table 1 is measured. In some embodiments, the status of one or more peptides in Table 1 is measured.

TABLE 1
Examples of Peptides as Cancer Markers

| GeneSymbol | phospho-sites | Peptide1 | Peptide2 | Peptide3 | Peptide4 |
|---|---|---|---|---|---|
| EGFR | non-phospho | GDSFTHTPPLDPQELDILK | IPLENLQIIR | | |
| | | (SEQ ID NO: 1) | (SEQ ID NO: 38) | | |
| ERBB2 | non-phospho | GLQSLPTHDPSPLQR | VLQGLPR | | |
| | | (SEQ ID NO: 2) | (SEQ ID NO: 39) | | |
| ERBB3 | non-phospho | ESGPGIAPGPEPHGLTNK | SLEATDSAFDNPDYWHSR | VLGSGVFGTVHK | |
| | | (SEQ ID NO: 3) | (SEQ ID NO: 40) | (SEQ ID NO: 64) | |
| ERBB4 | non-phospho | VLGSGAFGTVYK | EIPDLLEK | YLVIQGDDR | YSADPTVFAPER |
| | | (SEQ ID NO: 4) | (SEQ ID NO: 41) | (SEQ ID NO: 65) | (SEQ ID NO: 66) |
| PDCD1 | non-phospho | DFHMSVVR | GSADGPR | | |
| | | (SEQ ID NO: 5) | (SEQ ID NO: 42) | | |
| CD274 | non-phospho | DQLSLGNAALQITDVK | AEVIWTSSDHQVLSGK | | |
| | | (SEQ ID NO: 6) | (SEQ ID NO: 43) | | |
| MUC1 | non-phospho | QGGFLGLSNIK | NYGQLDIFPAR | | |
| | | (SEQ ID NO: 7) | (SEQ ID NO: 44) | | |
| MUC6 | non-phospho | TPVAHTTSATSSR | VTNEFVSEEGK | | |
| | | (SEQ ID NO: 8) | (SEQ ID NO: 45) | | |
| GRB7 | non-phospho | FTDLLQLVEFHQLNR | LIGQQGLVDGLFLVR | | |
| | | (SEQ ID NO: 9) | (SEQ ID NO: 46) | | |
| STARD3 | non-phospho | NNEYGDTVYTIEVPFHGK | EATAVVDQILAQEENWK | | |
| | | (SEQ ID NO: 10) | (SEQ ID NO: 47) | | |
| MTOR | non-phospho | TLDQSPELR | GYTLADEEEDPLIYQHR | | |
| | | (SEQ ID NO: 11) | (SEQ ID NO: 48) | | |
| TOP2A | non-phospho | HVDYVADQIVTK | GFQQISFVNSIATSK | | |
| | | (SEQ ID NO: 12) | (SEQ ID NO: 49) | | |
| PGAP3 | non-phospho | ESEDKFKLD | MAGLAAR | | |
| | | (SEQ ID NO: 13) | (SEQ ID NO: 50) | | |
| IFIH1 | non-phospho | VLLVEQLFR | ENKPVIPLPQILGLTASP | | |
| | | (SEQ ID NO: 14) | GVGGATK | | |
| | | | (SEQ ID NO: 51) | | |
| IFIT1 | non-phospho | VLDQIEFLDTK | SAIFHFESAVEK | | |
| | | (SEQ ID NO: 15) | (SEQ ID NO: 52) | | |
| LAP3 | non-phospho | GSDEPPVFLEIHYK | LYGSGDQEAWQK | | |
| | | (SEQ ID NO: 16) | (SEQ ID NO: 53) | | |
| MX1 | non-phospho | DVPDLTLIDLPGITR | ELPGFVNYR | | |
| | | (SEQ ID NO: 17) | (SEQ ID NO: 54) | | |
| MX2 | non-phospho | VLLEEGSATVPR | EITFFQTHPYFR | | |
| | | (SEQ ID NO: 18) | (SEQ ID NO: 55) | | |
| RELB | non-phospho | VARPPAAPELGALGSPDLS | SAGSILGESSTEASK | | |
| | | SLSLAVSR | (SEQ ID NO: 56) | | |
| | | (SEQ ID NO: 19) | | | |
| SAMD9L | non-phospho | SITVNDLTHLLEAAEK | SNFDETYIENVVR | | |
| | | (SEQ ID NO: 20) | (SEQ ID NO: 57) | | |
| STAT3 | non-phospho | GLSIEQLTTLAEK | VVENLQDDFDFNYK | | |
| | | (SEQ ID NO: 21) | (SEQ ID NO: 58) | | |
| TAP1 | non-phospho | QVAAVGQEPQVFGR | ELISWGAPGSADSTR | | |
| | | (SEQ ID NO: 22) | (SEQ ID NO: 59) | | |
| TAP2 | non-phospho | EIQDAVAR | QPNLPSPGTLAPTTLQGV | | |
| | | (SEQ ID NO: 23) | VK | | |
| | | | (SEQ ID NO: 60) | | |
| TAPBP | non-phospho | IHHPSLPASGR | FVPLPASAK | | |
| | | (SEQ ID NO: 24) | (SEQ ID NO: 61) | | |
| TNFAIP2 | non-phospho | VEALYELLR | QGLDGPPPTVEELK | | |
| | | (SEQ ID NO: 25) | (SEQ ID NO: 62) | | |
| TNFAIP3 | non-phospho | AVPLVNR | NWNDEWDNLIK | | |
| | | (SEQ ID NO: 26) | (SEQ ID NO: 63) | | |
| AKT1S1 | non-phospho | SLPVSVPVWGFK | | | |
| | | (SEQ ID NO: 27) | | | |
| ERBB2 | S1083 | SPLAPSEGAGsDVFDGDLG | | | |
| | | MGAAK | | | |
| | | (SEQ ID NO: 28) | | | |
| | | (this peptide also | | | |
| | | contributed: | | | |
| | | SPLAPsEGAGsDVFDGDLG | | | |
| | | mGAAK; | | | |
| | | (SEQ ID NO: 29)) | | | |
| ERBB2 | S1107 | GLQSLPTHDPsPLQR | | | |
| | | (SEQ ID NO: 30) | | | |
| ERBB2 | T1166 | PAGAtLER | | | |
| | | (SEQ ID NO: 31) | | | |
| ERBB2 | Y1127 | YSEDPTVPLPSETDGyVAP | | | |
| | | LTCSPQPEYVNQPDVRPQP | | | |
| | | PSPR | | | |
| | | (SEQ ID NO: 32) | | | |
| ERBB2 | Y1248 | GTPTAENPEyLGLDVPV | | | |
| | | (SEQ ID NO: 33) | | | |
| AKT1S1 | AKT1S1_S203 | sLPVSVPVWGFK | | | |
| | | (SEQ ID NO: 34) | | | |
| EIF4EBP1 | EIF4EBP1_S65 | FLMECRNsPVTK | | | |
| | | (SEQ ID NO: 35) | | | |
| AKT1S1 | AKT1S1_T266 | LNtSDFQK | | | |
| | | (SEQ ID NO: 36) | | | |
| SHC1 | SHC1_Y318 | ELFDDPSyVNVQNLDK | | | |
| | | (SEQ ID NO: 37) | | | |

phosphosite in lowercase, underlining indicates alternate possible residues

In some embodiments, the status of DNA and RNA molecular markers, or DNA and protein (including peptide) molecular markers, or RNA and protein (including peptide) molecular markers, or DNA and RNA and protein (including peptide) molecular markers are compared, and such proteogenomic analysis provides information for a medical practitioner to make a determination of diagnosis, prognosis, and/or treatment regimen.

In some embodiments, analysis of DNA, RNA, and/or protein may be used to determine the status, such as amounts, levels, presence, and/or absence of one or more immune cells in the biological sample. In some embodiments, analysis of DNA, RNA, and/or protein may be used to determine the status, such as amounts, levels, presence, and/or absence of one or more tumor infiltrating lymphocytes (TILs) in the biological sample. The analysis of DNA, RNA, and/or protein from the biological sample may measure specific DNA, RNA, and/or protein present in immune cells, including TILs.

Certain embodiments of the disclosure concern the prevention of misdiagnosis using methods encompassed herein. Current diagnostic methods, including immunohistochemistry and fluorescent in situ hybridization, may lack specificity, precision, and accuracy that may result in incorrect diagnosis, including incorrectly diagnosing an individual as having HER2+ cancer (for example). Embodiments disclosed herein may be utilized to diagnose accurately an individual, including an individual that was misdiagnosed or that is suspected of having been misdiagnosed. In some embodiments, methods disclosed herein are utilized to diagnose an individual that was misdiagnosed with HER2+ cancer with a different cancer, such as triple negative breast cancer. A skilled artisan practicing certain embodiments encompassed herein may alter the treatment regimen, dosage, or strategy administered to an individual, including based on the outcome of methods encompassed herein.

### VII. Microscaled Proteomics (MiProt)

Certain embodiments of the disclosure concern the analysis of one or more protein and/or peptide samples, which may be done by one or more methods encompassed herein, such as any microscaled proteomics (MiProt) method encompassed herein. The protein and/or peptide samples may be from any source, including from a biological sample such as a biological sample encompassed herein. In some embodiments, proteins are processed to peptides, and the peptides are tagged with one or more unique tags of known molecular weight. The peptides derived from each protein sample may be manipulated to have a unique tag, such that peptides are able to be identified based on the protein sample from which the peptides were derived. In some embodiments, after the peptides have been tagged, the tagged peptides are combined into at least one vessel. In some embodiments, the combined, tagged peptides may be subjected to sorting. Any method known in the art for sorting peptides may be used. Examples of methods for sorting peptides include chromatography, such as reverse-phase chromatography or basic reverse-phase chromatography, immunoprecipitation, affinity sorting, electrophoresis, or a combination thereof. Peptides may be sorted by size, charge, polarity, solubility, isoelectric point, affinity to other molecules, presence or absence of at least one posttranslational modification such as phosphorylation, or a combination thereof. In some embodiments, the tagged peptides are subject to mass spectrometry analysis. In some embodiments, the tagged and sorted peptides are subject to mass spectrometry analysis.

In some embodiments, including embodiments utilizing a MiProt method encompassed herein, peptides are measured using a selected reaction monitoring method, including parallel reaction monitoring, consecutive reaction monitoring, and multiple reaction monitoring.

In particular embodiments, the scale at which the methods are performed is greatly reduced compared to prior methods. The required amount of input sample is reduced by at least a factor of 2, 3, 4, 5, 6, 7, 8, 9, 10, 11, 12, 13, 14, 15, 20, 25, 50, 100, 250, 500, 1000, 5000, 10,000, and so forth by decreasing buffer and solvent amounts, minimizing exposure of sample to surface areas that could lead to sample loss, and scaling the number and method of fraction cancatenation of off-line bRP fractions for phosphopeptide enrichment from 12 fractions down to just 4.

### VIII. Methods of Treatment

Embodiments of the disclosure include methods of treatment for cancer, particularly for cancer that has been diagnosed or prognosticated based on analysis methods encompassed herein. The cancer of an individual may be treated following the outcome (and as a result of the outcome) of methods for analyzing DNA, RNA, and protein from a biological sample, and such treatment may be selected because of the analysis provided by that method. Specific methods for the analysis that results in the determination of an appropriate treatment (For example, targeted to HER2 or not targeted to HER2) may include sectioning of one or more regions from a biological sample (such as a biopsy) and combining a plurality of sections from different regions of the biological sample into multiple vessels, followed by isolating DNA from the plurality of sections in a first vessel, isolating RNA from the plurality of sections in a second vessel, and isolating protein from the plurality of sections in a third vessel, followed by analyzing the DNA, RNA, and protein (including in the form of peptides). In some cases, a treatment regimen is determined because of analysis of one or more protein samples in which the protein is digested into peptides that are tagged with a unique tag of known molecular weight to produce tagged peptides; combining all tagged peptides from different proteins; sorting the tagged peptides based on hydrophobicity; and subjecting the sorted peptides to LC-MS/MS.

In particular embodiments of the disclosure, method for treating an individual with at least one cancer marker-targeted treatment are encompassed in which there is determination whether the individual has cancer cells positive for the cancer marker by subjecting a biopsy from the individual to any analysis method encompassed herein. As a result of the analysis whether the individual has cancer cells positive for the cancer marker, in particular cases one of the following occurs: (a) administering a therapeutically effective amount of the cancer marker-targeted treatment to the individual who was determined to have cancer cells positive for the marker, or (b) not administering the cancer marker-targeted treatment to the individual who was determined not to have cancer cells positive for the cancer marker. In specific embodiments, the individual who was determined to have cancer cells positive for the cancer marker is further determined to have cancer cells susceptible to one or more other cancer marker-targeted treatments by waiting a particular duration of time (for example, 1, 2, 3, 4, 5, 6, 7, 8, 9, or 10 or more days) after administering the therapeutically effective amount of the cancer marker-targeted treatment; obtaining a new biological sample from the individual, and repeating an analysis method for cancer cells positive for the cancer marker. Such a process determines if the individual has a change in cancer marker status; and one of the following may then occur, in specific cases: (a) continuation of administering of the cancer marker-targeted treatment to the individual who was determined to have a change in molecular marker status, or (b) cessation of administering the cancer marker-targeted treatment to the individual who was determined not to have a change in molecular marker status. In specific cases of any method encompassed herein, the status change is defined as a change in the level, presence, absence, post-translational modification (including phosphorylation), or a combination thereof of one or more certain molecular markers. In certain cases, a therapeutically effective amount of a different cancer therapy is provided to the individual that was determined not to have a change in cancer marker status.

Embodiments of treatment methods include methods of distinguishing for an individual cancer marker-positive cells and/or tissue from cancer marker-negative cells and/or tissue, comprising subjecting cells and/or tissue from the individual to any method encompassed herein. In particular embodiments of the disclosure, there are methods of determining the susceptibility of an individual with cancer to a cancer treatment, comprising the step of subjecting a biological sample from the individual to any analysis method encompassed herein. When the individual is determined by the analysis method to be susceptible to the cancer treatment, a therapeutically effective amount of the cancer treatment is provided. Embodiments of the disclosure also encompass methods of detecting cancer marker status in the individual, followed by a suitable cancer treatment because of the detection.

In some embodiments, an individual is subjected to the treatment methods encompassed herein and based on the analysis methods encompassed herein but is also given another therapy, such as surgery, radiation, drug therapy, chemotherapy, hormone therapy, immunotherapy, or a combination thereof.

### EXAMPLES

The following examples are included to demonstrate particular embodiments of the disclosure. It should be appreciated by those of skill in the art that the techniques disclosed in the examples that follow represent techniques discovered by the inventors to function well in the practice of the disclosed methods and compositions, and thus can be considered to constitute particular modes for its practice. However, those of skill in the art should, in light of the present disclosure, appreciate that many changes can be made in the specific embodiments that are disclosed and still obtain a like or similar result without departing from the spirit and scope of the methods and compositions of the disclosure.

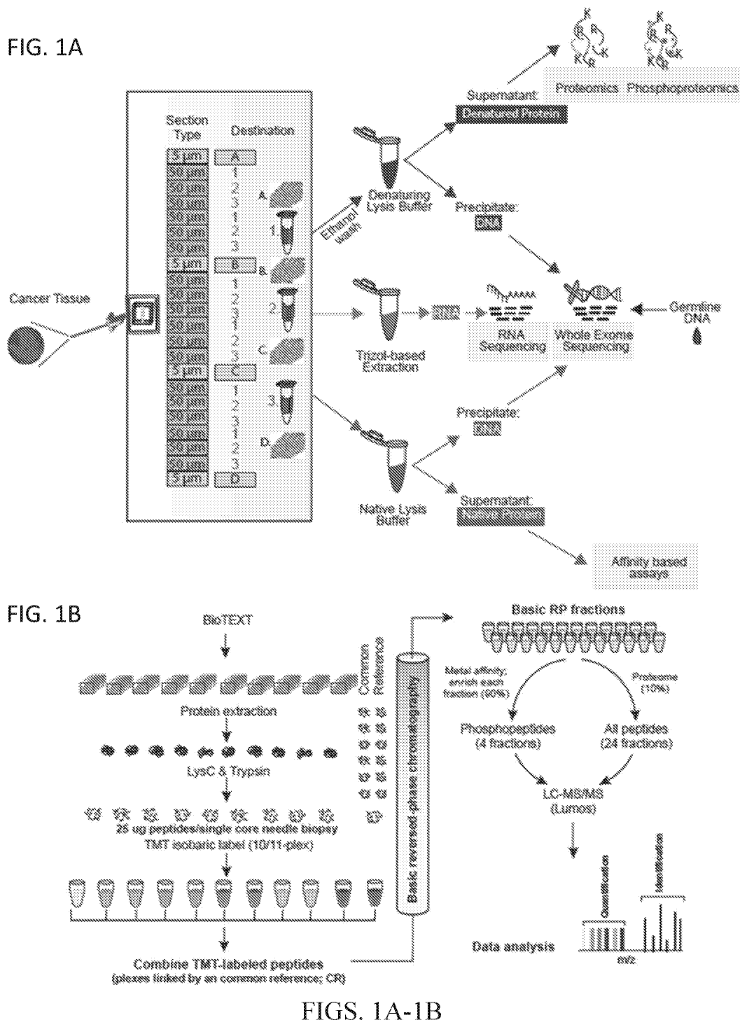

The examples of laboratory protocols described herein comprise two microscaling methods that provide preparative and analytical approaches with one or more of the following features (FIGS. 1A-1B): (1) the extraction strategy (referred to herein periodically as “BioTExt”) maximizes the yield of analytes from small clinical samples by isolating proteins for MS analysis before DNA extraction from the residual pellet; (2) the interposition of multiple 5 μm sections for histological analysis to provide tumor content information throughout the core biopsy; (3) a sectioning approach that ensures proteomic and genomic analyses are conducted on near identical biological replicates; (4) successful scaling of MS-based proteomics technology (referred to herein periodically as “MiProt”) for analyses of limited amounts of biopsy derived protein.

### Example 1

### Development and Evaluation of the Biopsy Trifecta Extraction Protocol (Biotext)

To perform proteogenomics analyses from flash-frozen diagnostic core needle biopsies, the BioTExt protocol was devised and optimized. A single optimal cutting temperature (OCT)-embedded core biopsy was serially sectioned with alternating 50 μm sections transferred into 3 different 1.5 ml tubes (FIG. 1A). A total of 6 sections were transferred into each tube. To assess sample quality, 5 μm sections were taken before the first and after every sixth 50 μm section for H&E staining, with adequate quality control requiring 50% average tumor content throughout the sample. The first tube was used to extract denatured protein and DNA, the second tube was used for RNA isolation, and the third tube was used to extract native protein and DNA. The denatured protein was subsequently used for proteomic and phosphoproteomic analyses described herein, and DNA and RNA were used for genomic analysis.

### Example 2

### Development and Evaluation of Microscaled Proteomics

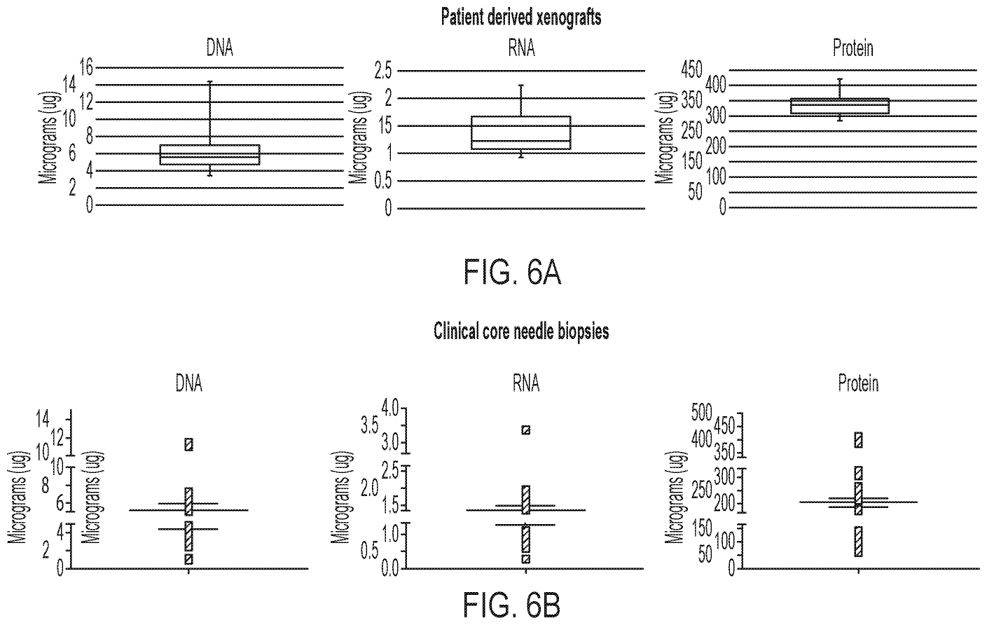

To assess the quantity of recoverable analytes using the procedure outlined above, BioTExt was applied to several OCT-embedded core-needle biopsies collected from a total of 4 previously established breast cancer patient-derived xenograft (PDX) models: WHIM2, WHIM14, WHIM18 and WHIM20¹¹. The yield for the sum of all six sections from a single biopsy in these PDX tumors ranged from 2.5-14 μg DNA, 0.9-2.3 μg RNA and 280-430 μg of protein. Extraction yields for the nucleic acid extractions are provided in FIG. 6A. The yields of the three analytes required a method capable of providing a deep-scale proteome and phosphoproteome despite lower analyte input. Because a wide range of needle sizes (14-22 gauge) are used to obtain diagnostic biopsies and different tumor types yield widely varying amounts of protein, a minimum of 25 μg of input peptide/sample was set as the target. This amount should reasonably and consistently be obtained from six 50 μm curls from a needle core biopsy even in low-yield tumors or when using small biopsy gauges. BioTExt also allows additional sections from each core to be reserved for verification studies, such as targeted MS analyses once candidate proteins and phosphosites of interest have been identified or for replication of full-depth discovery analyses if required due to technical failures.

To obtain deep proteome and phosphoproteome coverage from 25 μg of input peptide/sample, a tandem mass-tagging (TMT) peptide labeling approach was employed¹² (FIG. 1B). Since the mass tags are isobaric, signals from the same peptides in each sample stack at the MS1 level, improving overall sensitivity for identification and quantification, a key advantage for the analysis of small amounts of protein. Multiplexing also increases sample analysis throughput by 10-fold relative to label-free approaches. Successful microscaling required several modifications to the bulk-optimized CPTAC workflow 8 to allow labeling, fractionation and analysis of low amounts of proteins. This overall method is referred to herein periodically as “Microscaled Proteomics” or “MiProt”.

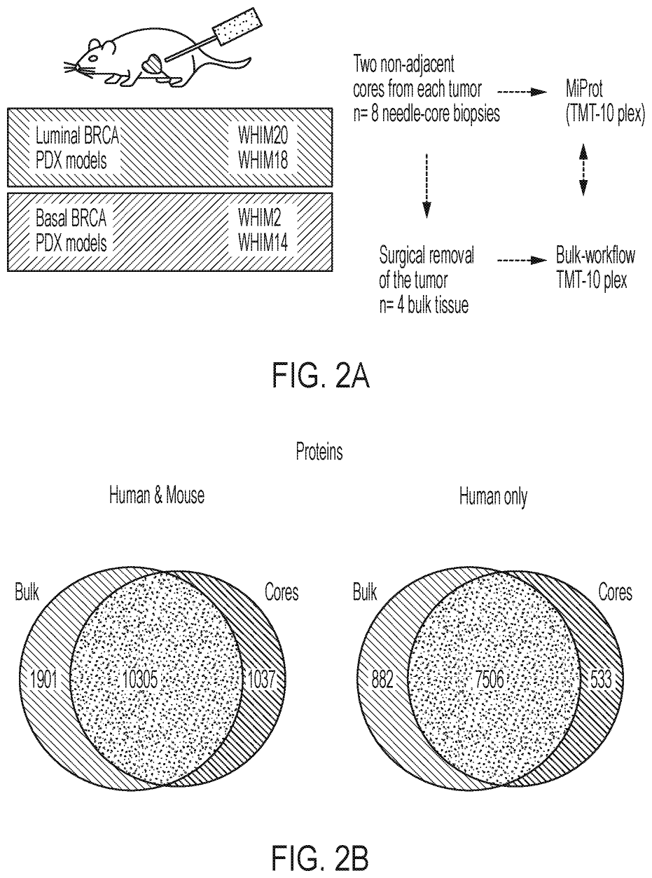

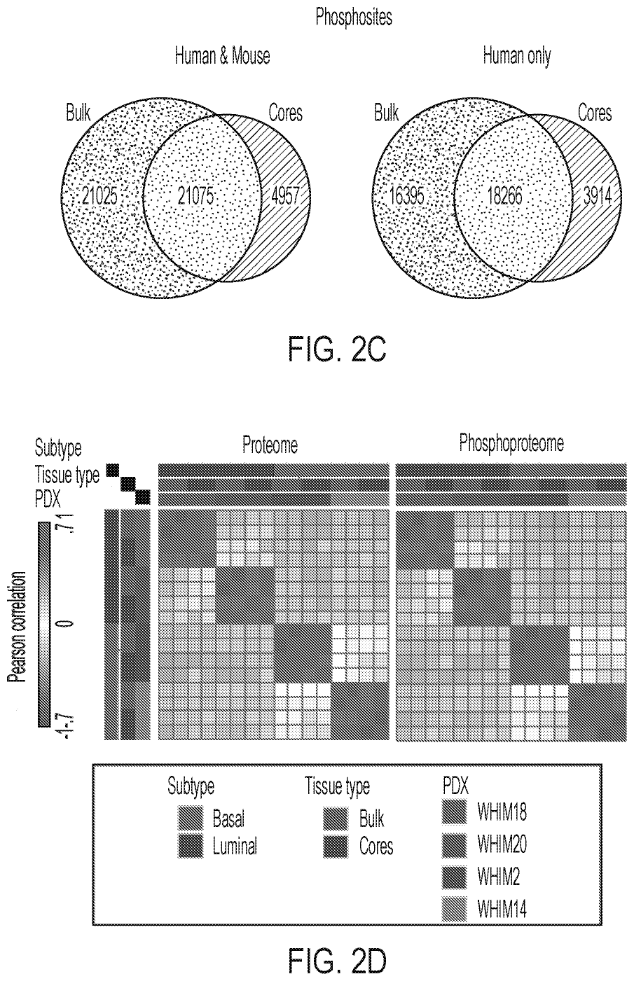

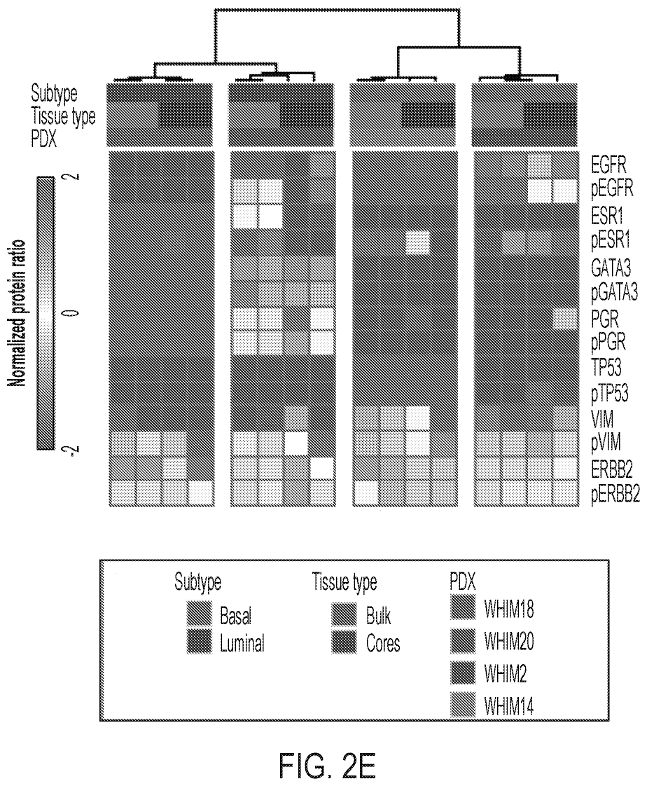

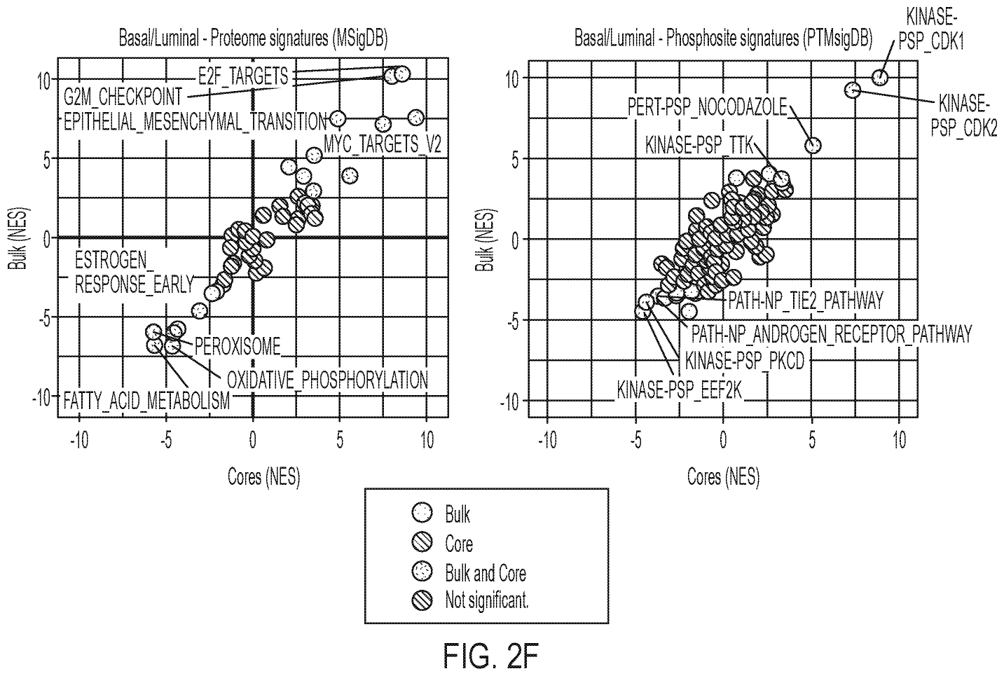

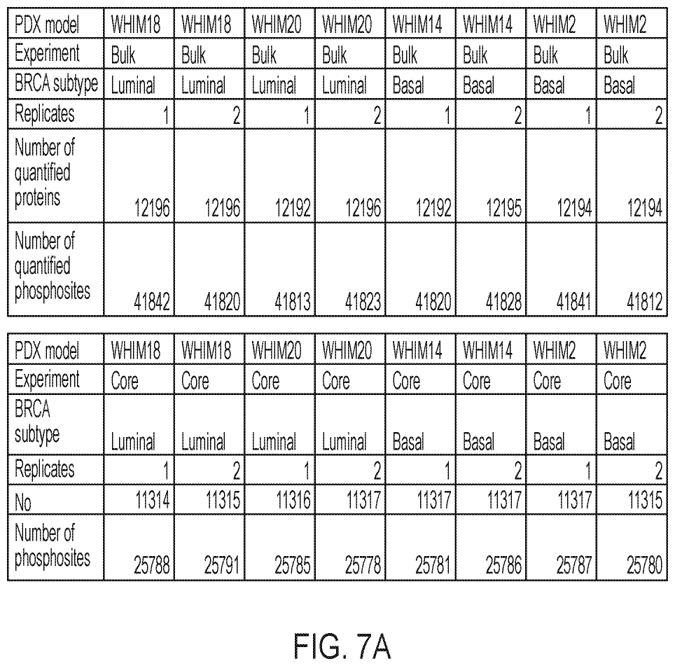

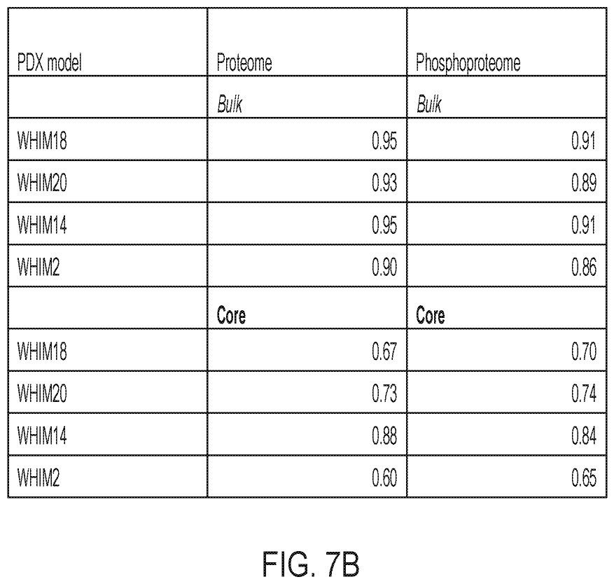

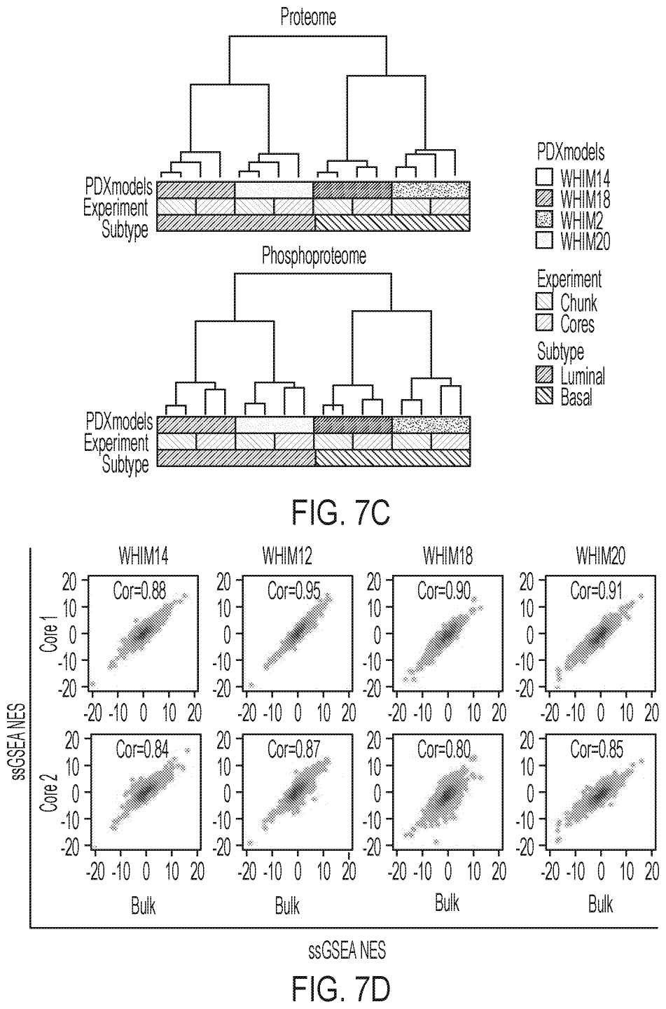

To determine if the proteomic coverage for core-needle biopsies are comparable to those obtained using a workflow optimized for bulk tumors (the Clinical Proteomics Tumor Analysis Consortium (CPTAC) workflow) 8, a head-to-head comparison experiment utilizing previously published breast cancer PDX models was executed¹¹, including two luminal (WHIM18 and WHIM20) and two basal-like models (WHIM2 and WHIM14) (FIG. 2A). To simulate a diagnostic analysis, two needle-biopsy cores were collected from each PDX model. The cored xenograft tumors were then surgically removed for analysis of the residual bulk material. The cores were OCT-embedded, flash frozen and subjected to BioTExt followed by MiProt. The remaining bulk tumors were flash frozen and cryopulverized, followed by analysis using the original CPTAC workflow^{8,13}. Totals of 300 μg of peptides per sample were analyzed with the original and 25 μg of peptides per sample with the MiProt workflow using a randomized experimental layout. Protein and phosphopeptide expression were reported as the log ratio of each sample's TMT intensity to the intensity of an internal common reference included in each plex. Both workflows identified more than 10,000 proteins, of which >7,500 were identified as human. Extensive overlap was observed between the populations of proteins identified by the two workflows (FIG. 2B). MiProt identified >25,000 phosphosites from each core, and these sites showed substantial overlap with those identified by high input, bulk workflows (FIG. 2C, FIG. 7A). Particularly notable is that an equivalent number of quantified proteins and more than half the number of quantified phosphosites per sample were identified despite using 12-fold less tumor material in MiProt vs conventional bulk analyses. There was a high correlation of TMT ratios between replicates of bulk tumors and between replicates of cores across all 4 PDX models for both the proteomics and phosphoproteomics data (FIG. 2D, FIG. 7B). In addition to a high degree of overlap in protein and phosphosite identities, expression was also highly correlated (R >0.65) between cores and bulk for individual PDX models, as can be visualized by the close juxtaposition of core and bulk samples from the same PDX model upon unsupervised hierarchical clustering (FIG. 7C).

Expression profiles of key basal and luminal markers, showed some degree of heterogeneity, although, showed a comparable trend overall, between bulk and core analysis (FIG. 2E) at the levels of both the proteome and phosphoproteome for all PDX models except for WHIM20, where phosphorylated EGFR, phosphorylated PGR and ESR1 protein showed reduced expression in cores relative to bulk, suggesting that there may be some heterogeneity in this particular PDX model as shown previously¹¹. By contrast, ERBB2, a breast cancer marker that should not be highly expressed in these clinically ERBB2− (no ERBB2 amplification) cases showed more uniform expression across the different PDX models. Overall, cores provided proteomics data that yielded results consistent with those obtained from global expression profiles from bulk tissue.

To address whether differentially regulated pathways and phosphopeptide-driven signaling in luminal versus basal subtypes were captured by the microscaled workflow, pathway-level and kinase-centric analyses were applied to the bulk and core sample data. Single-sample gene-set enrichment analysis (ssGSEA) was applied to proteomics data, and post-translational modification set enrichment analysis (PTM-SEA) to the phosphoproteomic data^{14,15}. The luminal-basal differences captured by bulk tissue analysis were highly correlated with differences detected using cores for both protein and phosphopeptide expression (FIG. 2F). Of note, the data recapitulates previously observed luminal-basal differences, which provided a quality metric for the proteomics dataset both for cores and bulk tissue 2,6. The same conclusion was reached in bulk versus core comparisons performed on the normalized TMT protein ratios for individual PDX models (FIG. 7D). Despite identifying ˜40% fewer phosphorylation sites, most of the differential Luminal-Basal kinase signatures identified in the bulk tissue were captured by MiProt (FIG. 2F, right).

### Example 3

### Application of Microscaled Proteogenomic Analyses to Clinical Core Biopsies from Patients Treated for ERBB2+ Locally Advanced Breast Cancer

The effectiveness of the BioTExt and MiProt analyses in PDX models encouraged the application of these methods to clinical tumor samples acquired in the context of a small-scale ERBB2+ breast cancer study (Discovery protocol 1 (DP1); NCT01850628). This study was designed primarily to investigate the feasibility of proteogenomic profiling before and immediately after initiating trastuzumab-based treatment for ERBB2+ breast cancer. Patients with a palpable breast mass diagnosed as ERBB2 positive breast cancer by a local laboratory were treated at the physicians' discretion, typically with trastuzumab in combination with pertuzumab and chemotherapy. The regimens included docetaxel or paclitaxel, the former often combined with carboplatin. The protocol (see Clinical Trial NCT01850628 at the Clinical Trials website of the NIH) was designed to study acute treatment perturbations by accruing OCT-embedded core needle biopsies before and 48 to 72 hours after treatment (referred to pre-treatment and on-treatment, respectively, throughout the text).

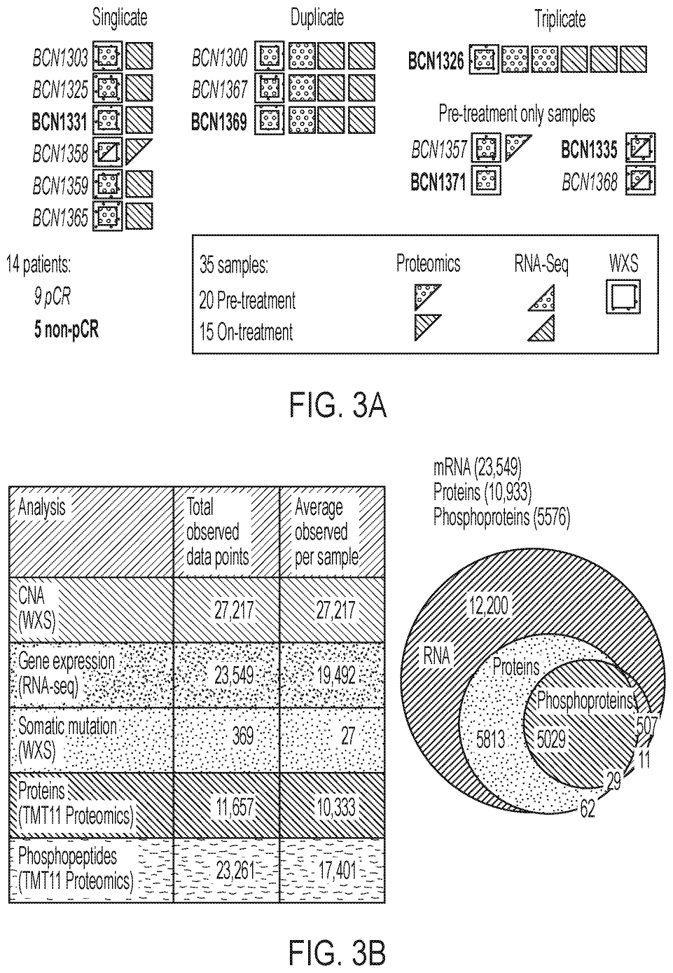

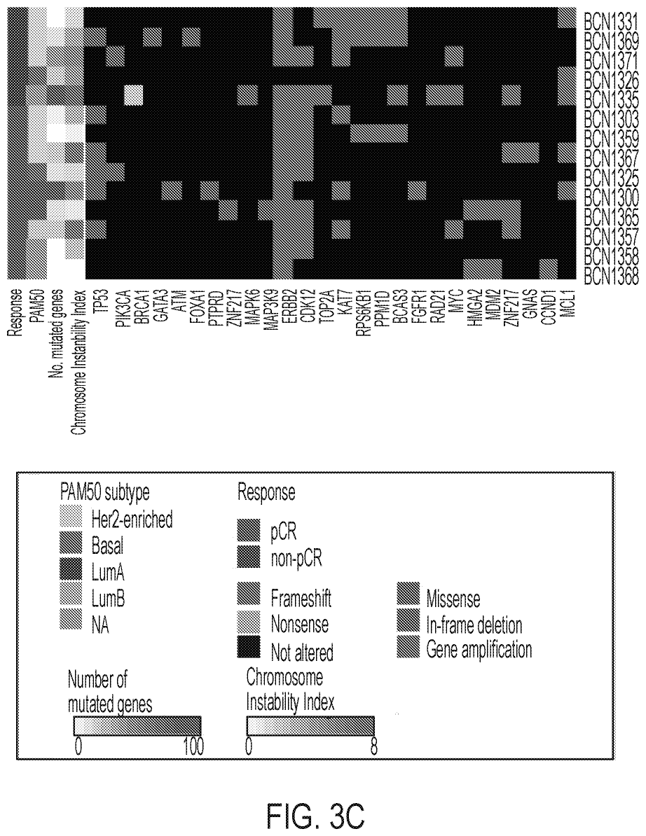

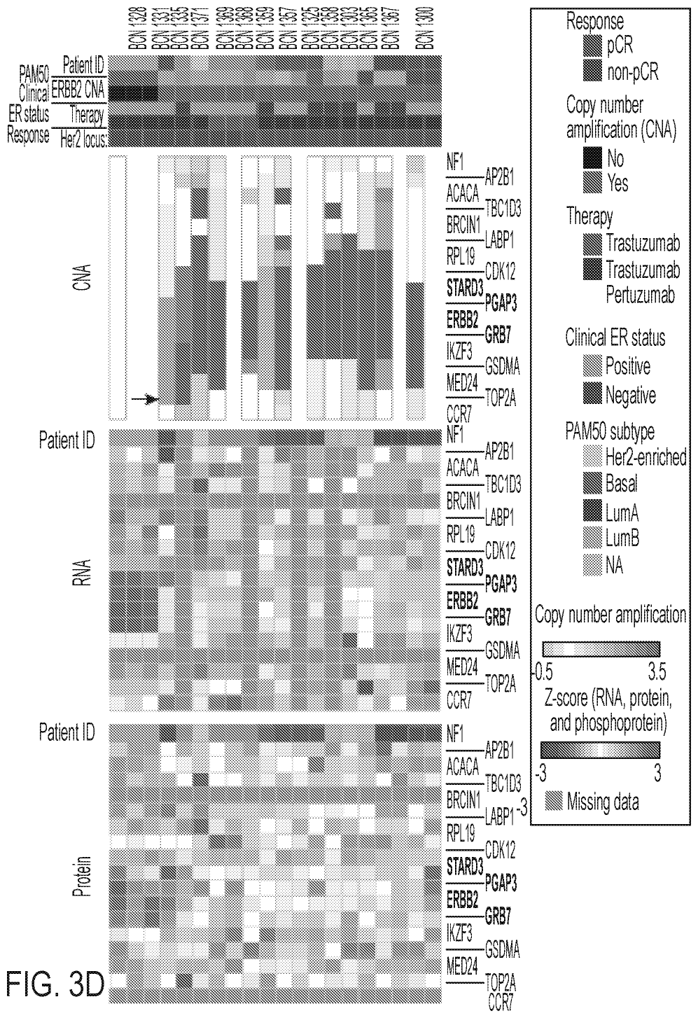

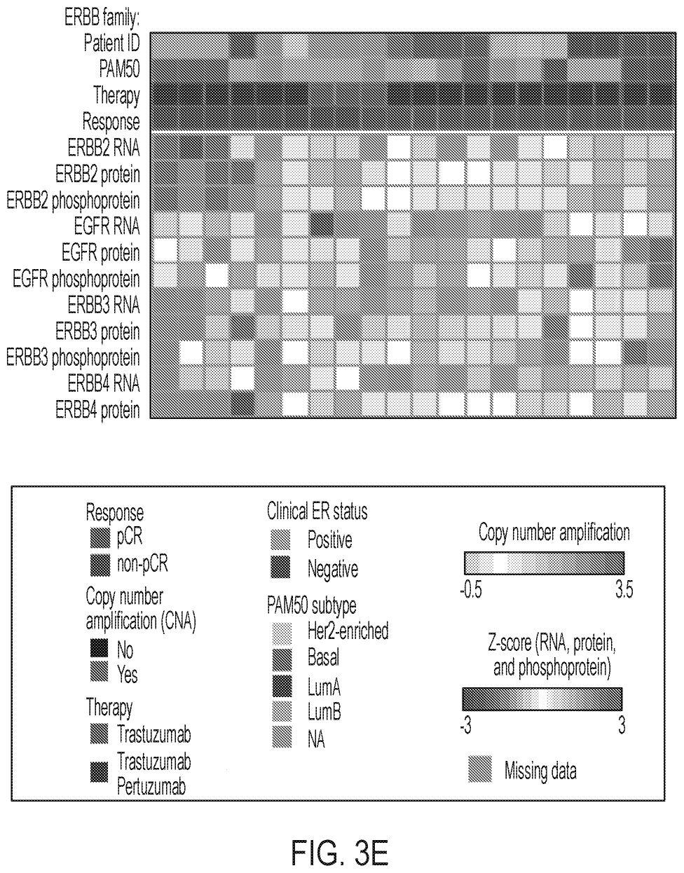

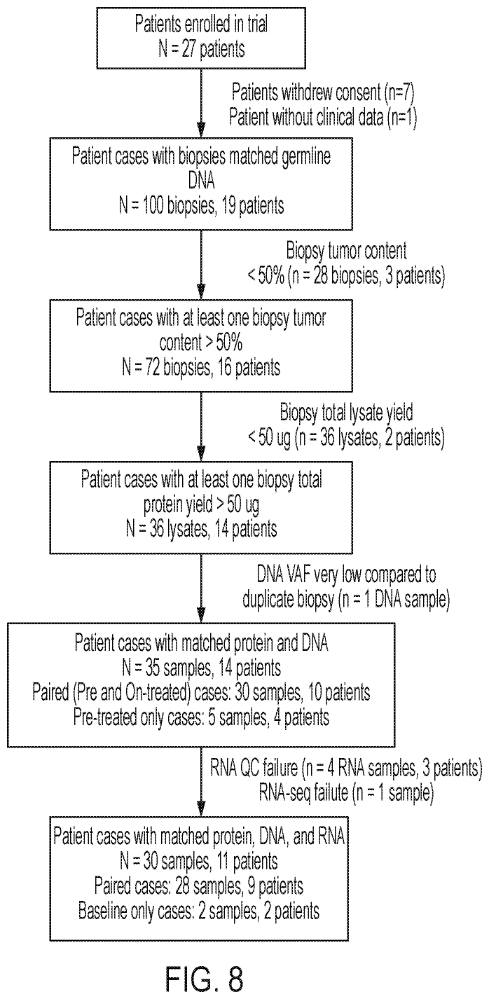

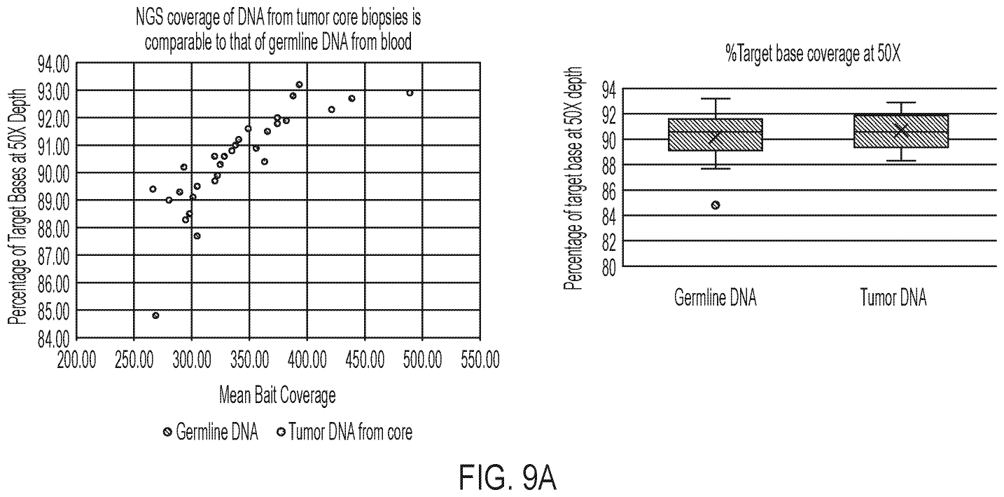

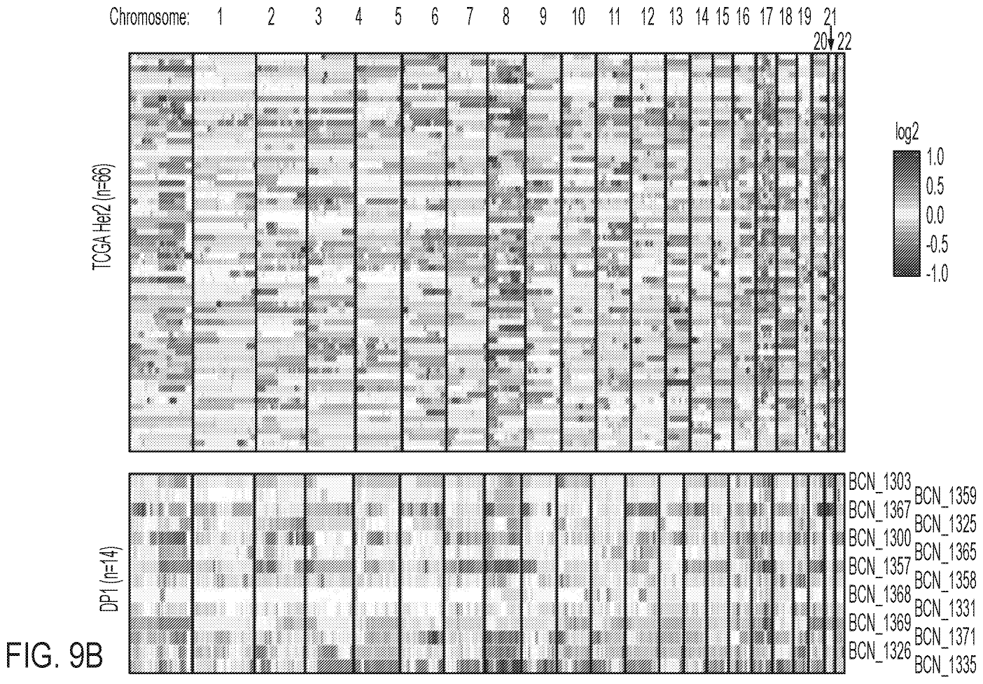

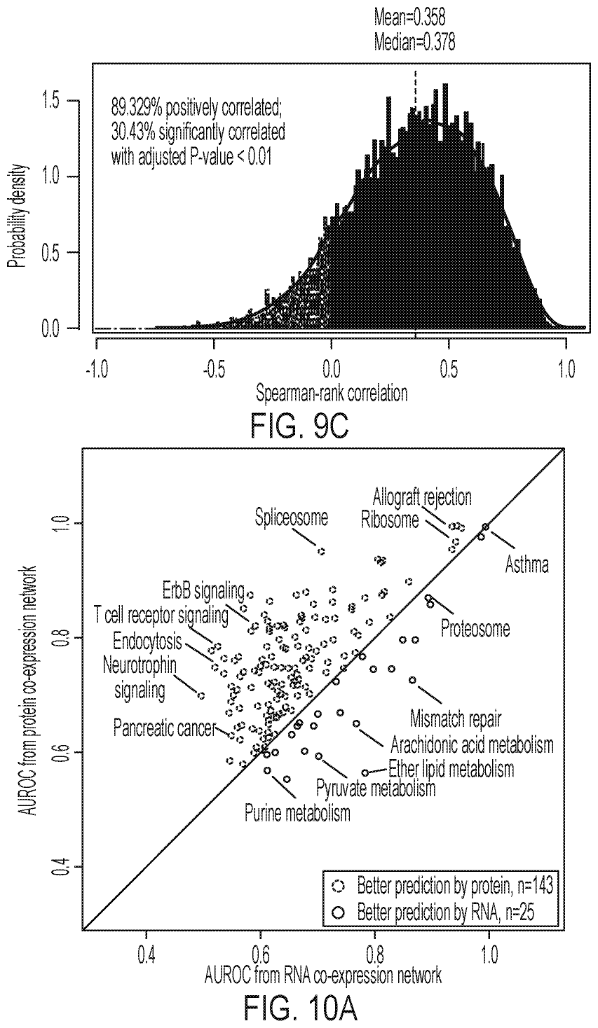

As shown in the REMARK (Reporting Recommendations for Tumor Marker Studies)¹⁶ diagram (FIG. 8), core biopsy samples were available from 19 patients. Proteogenomic analysis could be conducted on samples from 14 patients as five cases had low tumor content biopsies. For the included samples, the analyte yield varied across different cores, but the lower-range yields of DNA, RNA and protein (0.4 μg, 0.2 μg, and 45 μg respectively) were sufficient to demonstrate the suitability of the optimized extraction protocol for clinical biopsy specimens (FIG. 6B). Protein, and RNA when available, was also analyzed for on-treatment cores from 10 patients, with analysis of duplicate pre- and on-treatment cores achieved in four of the patients, and of triplicate cores in one patient (FIG. 3A). In total, 35 cores were analyzed. Tumor and germline whole-exome sequencing was performed using DNA from a single baseline core for all 14 patients. DNA isolated from cores using BioTExt yielded target coverage comparable to that from genomic DNA isolated from blood (generated using standard organic extraction techniques), indicating there was no compromise in DNA quality when a protein-sparing technique was used (FIG. 9A). Transcriptome analysis was successful for 30 cores with available RNA, corresponding to 11 of the 14 patients, and TMT 11-plex-based proteomics analysis was performed for all 35 cores.

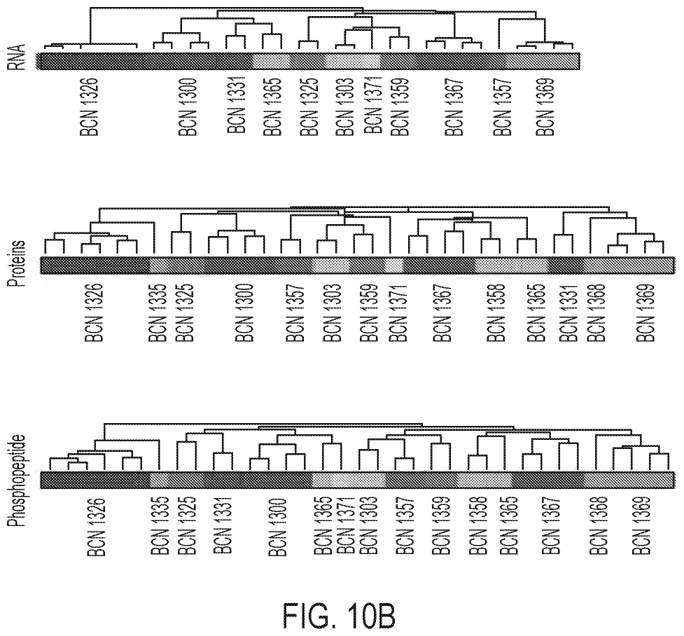

On average, copy number information on >27,000 genes, measured mRNA transcripts for >19,000 genes, and quantified >10,000 proteins and >17,000 phosphosites was obtained from each individual patient sample, with a large overlap of gene identification across different datasets (FIG. 3B). This was equivalent to the depth obtained in previous large-scale breast cancer proteogenomics efforts based on bulk tissues, with the exception of the phosphoproteome coverage, which achieved about half of the number of sites previously reported for tumor bulk-level characterization^{2,17}. For 13 out of the 14 cases, ERBB2 amplification was confirmed by exome sequencing along with amplifications and mutations in a range of genes previously implicated by the TCGA and ICGC breast cancer studies (FIG. 3C)^{18,19} including mutations in TP53 in 6 out of the 15 patients, PIK3CA missense mutations in BCN1371 and BCN1325, and a somatic nonsense mutation in BRCA1 in BCN1335. Also observed was a similar overall pattern of chromosomal amplifications and deletions between patients in this clinical trial and the subset of ERBB2+ tumors from TCGA, including a high frequency of previously described amplifications of chromosomes 1, 8, and 20 (FIG. 9B)¹⁹. The median gene-wise Spearman correlation between mRNA and protein across patient cores was 0.38, consistent with previous bulk-focused proteogenomics studies (FIG. 9C)^{1,2,4,17} In addition, co-expression networks derived from MiProt protein expression better predicted KEGG pathway function than those derived from mRNA expression for a similar proportion of pathways as previously reported for the published CPTAC breast cancer cohort²⁰ (FIG. 10A). Deep-scale proteogenomic analysis of duplicate and pre- and on-treatment cores from the same patient allowed an assessment of BioTExt sample processing reproducibility. Unsupervised hierarchical clustering based on 500 most-variable genes resulted in all duplicate cores clustering together at the level of mRNA, protein and phosphopeptide expression with the exception of samples from case BCN1365, where pre- and on-treatment profiles did not cluster together at the level of phosphopeptide expression (FIG. 10B). In summary, the data generated using microscaled methods was of comparable quality to previous bulk tissue-focused proteogenomics reports and yielded observations that were consistent with the expected biology for tumors diagnosed as ERBB2+.

### Example 4

### Proteogenomic Analysis of the ERBB2 Locus Suggests False Positive Clinical Diagnoses

All the patients in this study were locally diagnosed as ERBB2+ based on standard fluorescence in situ hybridization (FISH) and immunohistochemistry (IHC)-based approaches. A pathological Complete Response (pCR) occurred in 9/14 cases (64%), but 5 patients had residual cancer at surgery (non-pCR). To probe the possibility that some of the non-pCR cases were due to misassignment of ERBB2 status, proteogenomic analysis of the region of chromosome 17q spanning the ERBB2 locus and adjacent genes was performed (FIG. 3D). Most obviously, exome sequencing of BCN1326 did not show amplification of ERBB2 or other nearby genes (FIG. 3D, upper panel) and exhibited markedly lower levels of ERBB2 RNA (FIG. 3D, middle panel) and protein expression (FIG. 3D, lower panel) than pCR cases, suggesting a false positive. Additionally, expression levels from genes immediately flanking ERBB2 (STARD3, PGAP3 and GRB7, highlighted in red in FIG. 3D) were lower than pCR cases. BCN1331 and BCN1335 may represent a more subtle form of false positivity. While these samples showed a gain of ERBB2 copy number, ERBB2 protein levels remained low, similar to BCN1326. BCN1335 also showed greater absolute amplification of TOP2A than of ERBB2 (see black arrow FIG. 3D upper panel), and the TOP2A protein was markedly over-expressed compared to all other cases (FIG. 3D lower panel, of note the RNA analysis failed in this sample). This suggests that TOP2A was the more likely driver in this case.

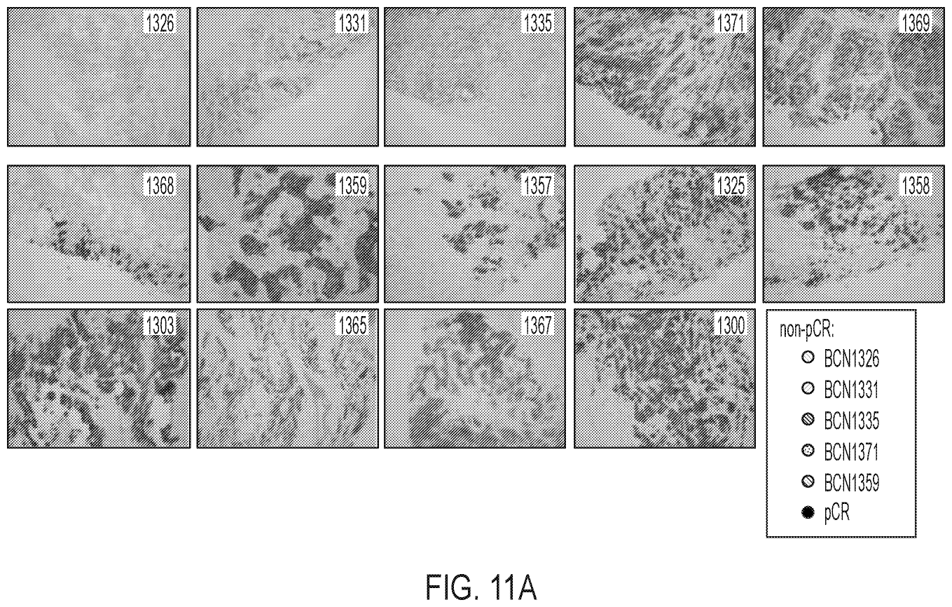

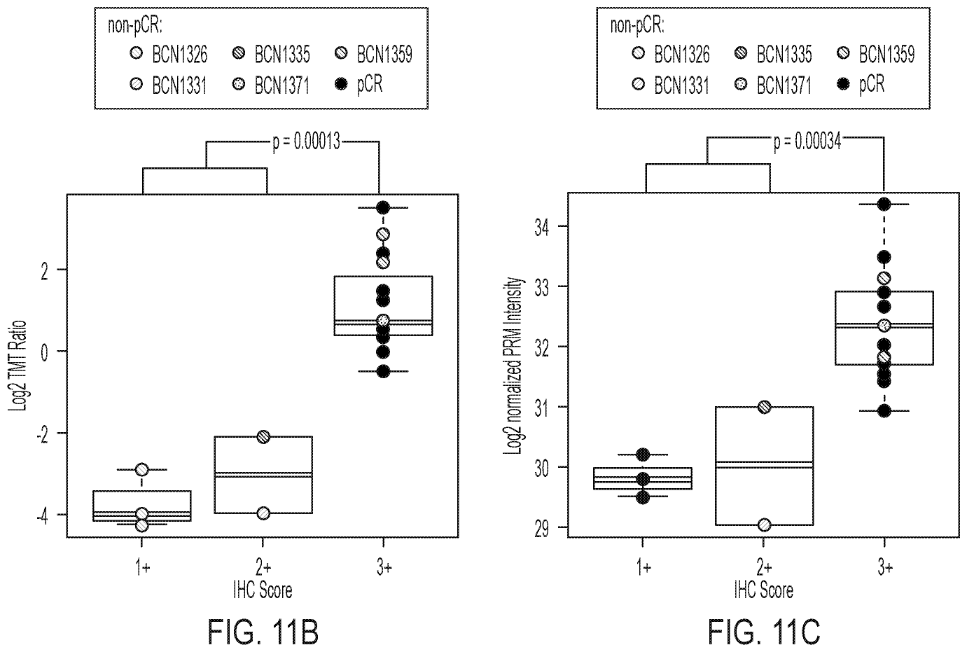

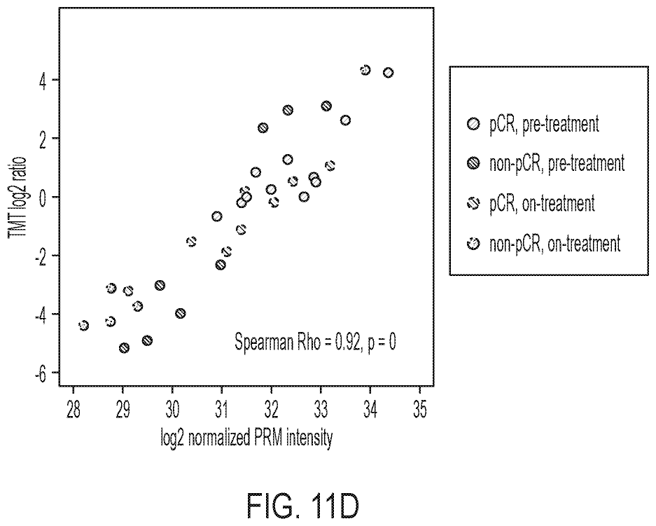

Levels of STARD3, PGAP3 and GRB7 both for RNA (BCN1331) and protein (BCN1331 and BCN1335) were also low, indicating that the amplicon may not drive sufficient ERBB2 expression for treatment sensitivity. When comparing these three false positive samples as a group with the nine “true” ERBB2 positive pCR cases, both the arithmetic mean for STARD3, ERBB2 and GRB7 protein log TMT ratios and the protein log ratios of each gene separately were significantly lower in the proposed false ERBB2 positive cases (mean: p=0.0114, STARD3: p=0.0255, ERBB2: p=0.0073, GRB7: p=0.0399). Protein levels of ERBB2 dimerization partners ERBB3 and ERBB4, as well as phospho-ERBB3, were also significant under-expressed in all three proposed false positive-samples when compared to the pCR cases (ERBB3 protein: p=0.0097, ERBB3 phosphoprotein: p=0.0318, ERBB4 protein: p=0.0131) (FIG. 3E). In contrast, protein and phosphoprotein levels of EGFR, the remaining dimerization partner of ERBB2, does not appear to be correlated with ERBB2 levels and was high in the non-pCR samples, suggesting that EGFR homodimers may play a driver role in signaling when ERBB2 is low^{21,22}. Thus, BCN1335 and BCN1331 represent “pseudo” ERBB2 positive cases, i.e. false ERBB2 positive cases for which proteogenomic evidence reveals insufficient ERBB2 expression to attain pCR despite gains in ERBB2 gene copy number. A central immunohistochemistry analysis indicated that ERBB2 was 1+ in BCN1326 and 2+ in BCN1335 and BCN1331, all the pCR cases were assigned 3+ staining (FIG. 11A). In contrast to the three ErbB2 low-expression cases, BCN1371 and BCN1369 were both non-pCR cases despite exome-confirmed ERBB2 amplification, ERBB2 RNA and protein expression similar to pCR cases with IHC 3+ ERBB2 staining and over-expression of STARD3, PGAP3, GRB7, ERBB3 and ERBB4 (FIG. 3D). These two cases therefore represent examples of true positive cases with intrinsic therapeutic resistance. Supporting the quantitative potential of microscaled proteomics, the samples with an IHC score of 3+ in a central assay showed significantly higher levels of ERBB2 expression than tumors scored 1+ or 2+ (p=0.00013). Parallel reaction monitoring (PRM) was also deployed as an orthogonal label-free protein quantification method on the same samples with an excellent correlation (R=0.92, p value=0) between the TMT and PRM-based MS approaches (FIG. 11B-FIG. 11D).

### Example 5

### Phosphoproteomic Analyses of Acute on-Treatment Samples Demonstrates the Feasibility of Pharmacodynamic Monitoring for Response Prediction

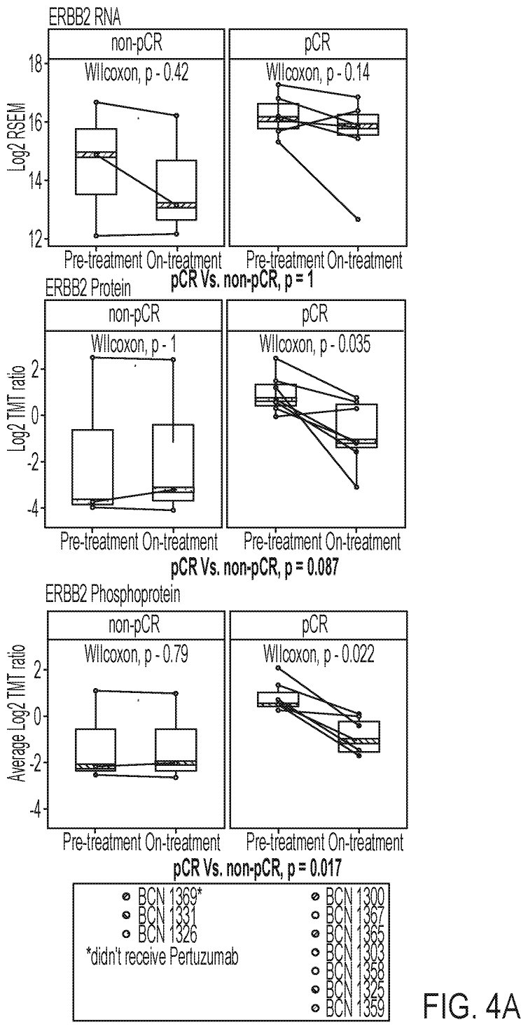

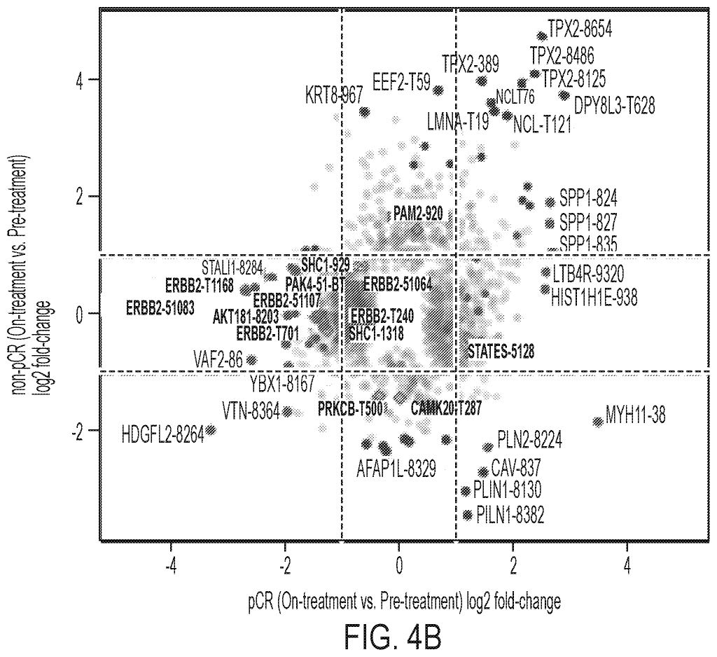

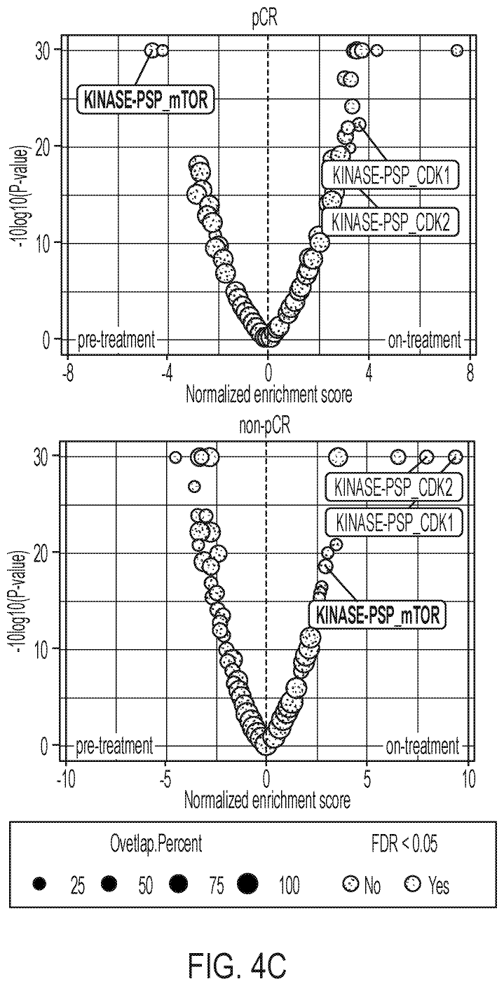

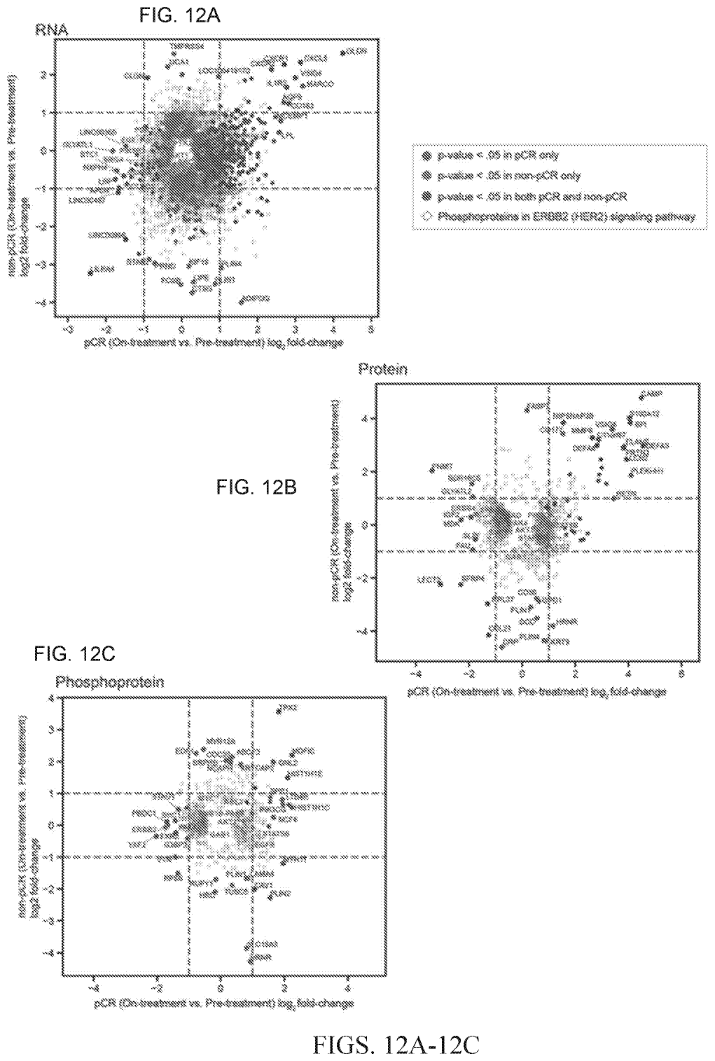

The DP1 clinical study was primarily designed to test the feasibility of phosphoproteomic analysis to identify early markers for responsiveness to ERBB2-directed monoclonal antibody therapy. Proteomics, Phosphoproteomics and RNAseq (for comparison) was therefore conducted on pre- and on-treatment core biopsies for nine patients with pCR and three patients without pCR. Differential treatment-induced changes were not observed at the RNA level and trended but did not reach significance at the ERBB2 protein level (FIG. 4A; while the ERBB2 protein levels showed significant reduction in pCR cases (p=0.031), the p-value for the comparison of this reduction between pCR and non-pCR cases was 0.067). However, greater downregulation of ERBB2 phosphoprotein (mean of all ERBB2 phosphopeptides) levels after 48-72 hours in pCR cases than in non-pCR cases was observed (two sample sum rank test, p=0.017; FIG. 4A). To explore these data further, limma²³, a more advanced statistical method specifically designed for differential expression analysis of small sample size studies, was employed. Differential ERBB2 RNA expression was again not seen for any comparison (FIG. 12). However, there was significant pCR-specific downregulation for both ERBB2 protein (limma: p=0.002 for pCR, p=0.63 for non-pCR, and p=0.029 for pCR vs. non-pCR) and phosphoprotein levels (p=0.000014 for pCR, p=0.88 for non-pCR, and p=0.0086 for pCR vs. non-pCR). More importantly, differential analysis of individual phosphorylation sites revealed pCR-specific significant downregulation of several phosphosites on proteins from the pathway, including sites on ERBB2 and SHC1, an adaptor that binds to ERBB2²⁴ (FIG. 4B). Most of the significant changes that were at least 2-fold and that affected the ERBB2 pathway were observed in the site-level phosphoproteomics data (FIG. 4B; FIG. 12).

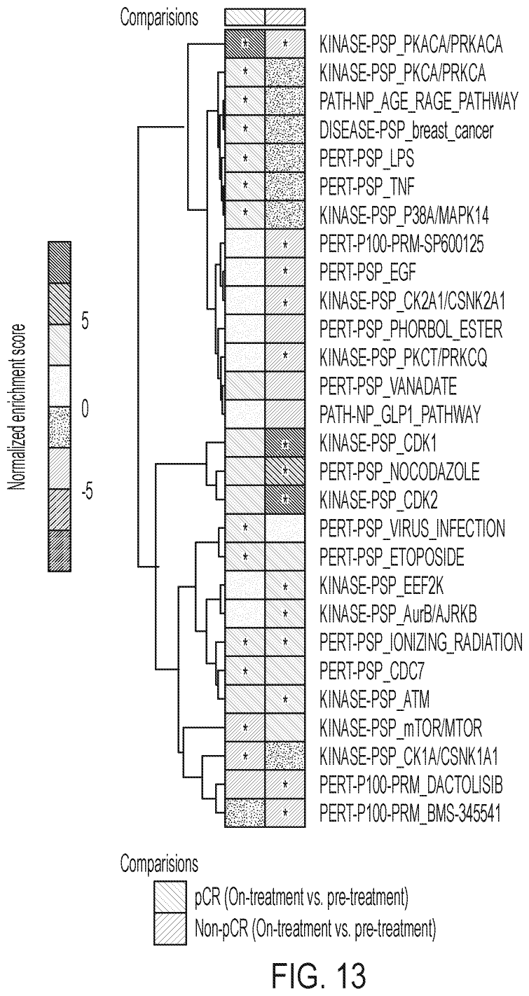

Given the well-understood kinase signaling cascades downstream of ERBB2²¹, a recently published tool for pathway analysis of phosphosites, PTM-SEA, was applied to the phosphoproteomics data¹⁴. This program uses a manually curated post-translational modification site database, PTMsigDB (see the GitHub website of Broad Institute), to estimate the activity for phosphoproteomics signatures resulting from chemical or genetic manipulation of a pathway or for kinases by analyzing signatures for target substrates with validated biochemical evidence. FIG. 4C shows significant phosphosite signatures for the comparisons tested (on- vs pre-treatment changes in pCR only and in non-pCR only). FIG. 13 shows a heatmap of phosphoproteome driven signatures that were significantly differentially regulated (FDR <0.05) upon treatment in either of the two groups. While the inferred activities of CDK1 and CDK2 kinases (KINASE-PSP_CDK1, KINASE-PSP_CDK2) were upregulated in the non-pCR patients, downregulation of mTOR activity (KINASE-PSP_mTOR) was most prominent exclusively in pCR cases upon treatment. This comparison of pre- and on-treatment samples thus suggests that in vivo downregulation of mTOR signaling, downstream of ERBB2, during treatment leads to a more favorable response.

### Example 6

### Pathway Enrichment Analysis Reveals Variability in the Tumor Immune Microenvironment and Other Putative Response Features in Individual Non-PCR Cases

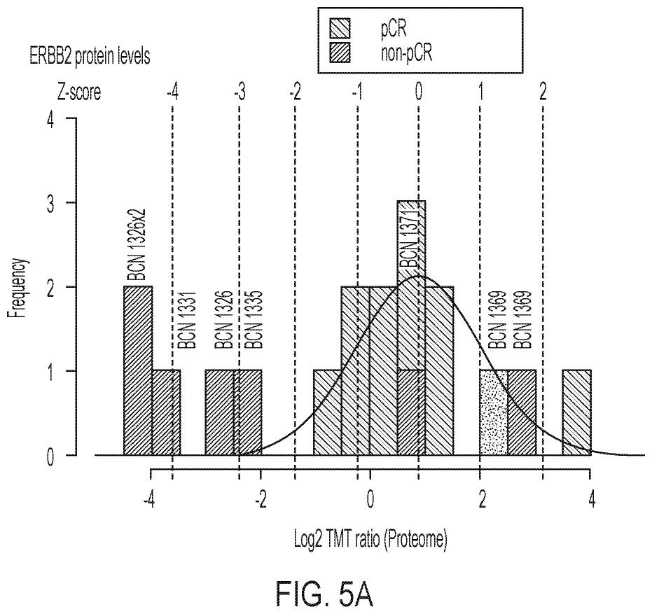

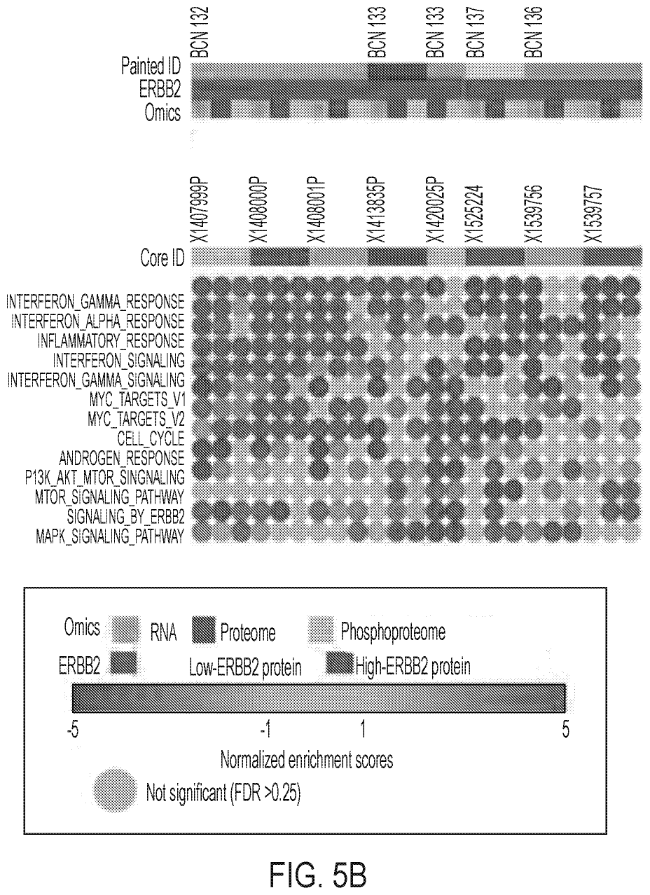

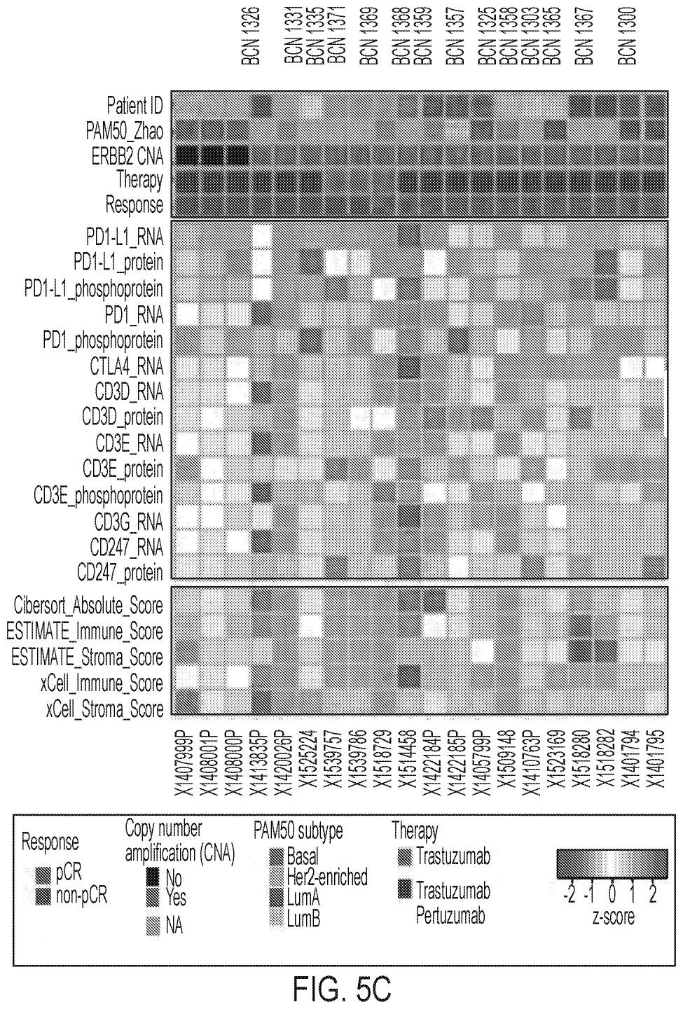

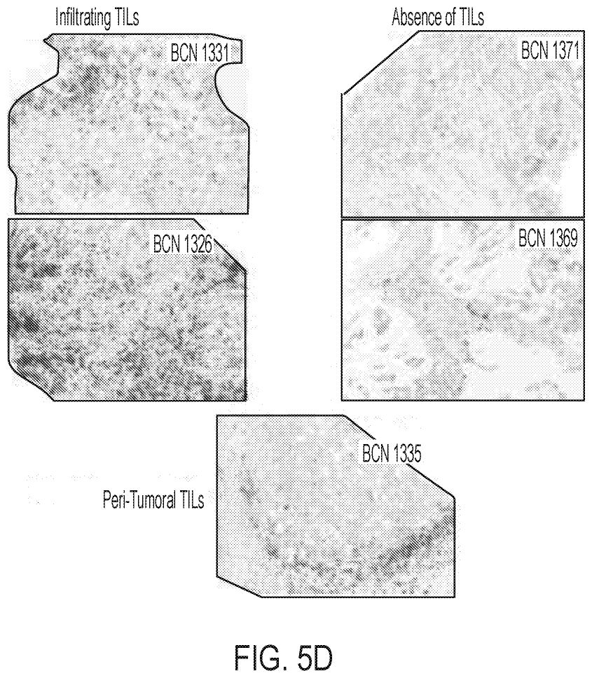

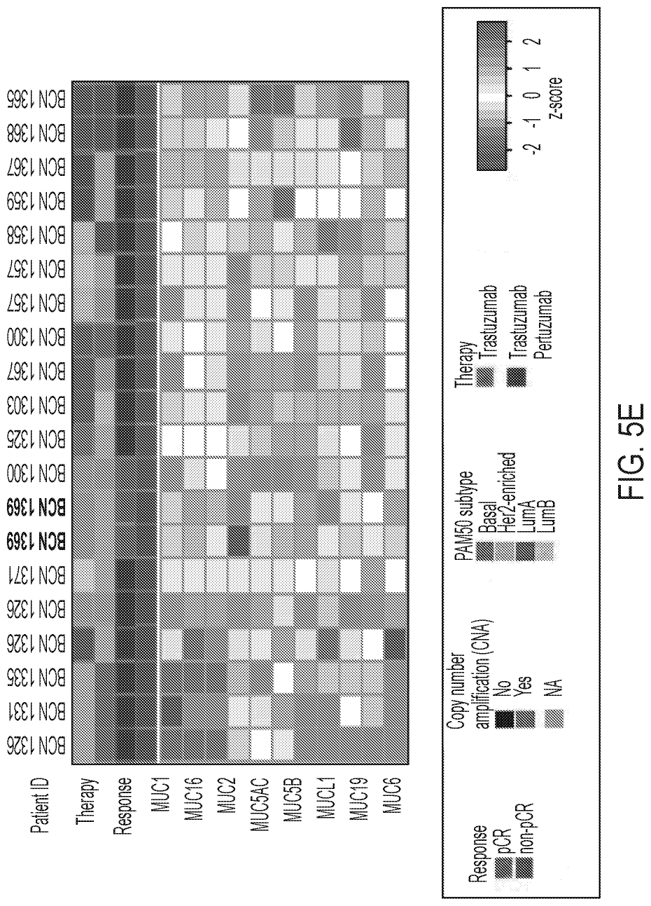

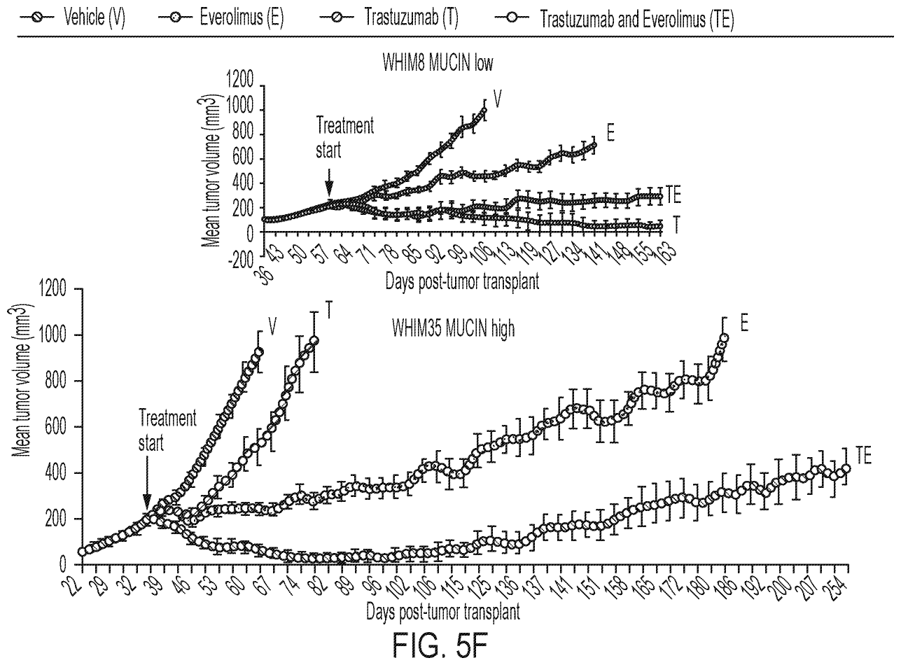

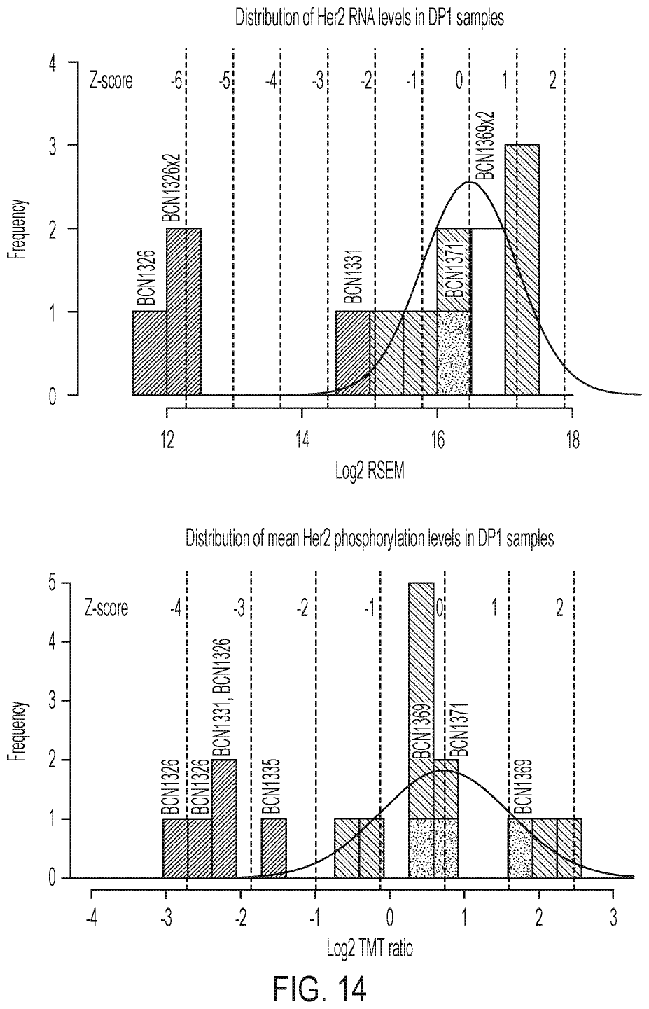

To explore biological processes that may contribute to inadequate response to therapy in non-pCR cases, RNA, protein and phosphoprotein outlier analyses on data from each pre-treatment core from the non-pCR cases with respect to the set of pre-treatment pCR cores was performed. Specifically, Z-scores were calculated for each gene/protein in a given individual non-pCR core relative to the distribution established from all of the pre-treatment pCR cores. The Z-scores of ERBB2 protein expression in non-pCR cases were consistent with the observations noted above; ERBB2 RNA, protein and phosphoprotein levels in patients BCN1326, BCN1331 and BCN1335 were outliers with negative Z-scores while ERBB2 expression in patients BCN1369 and BCN1371 lied within the normal distribution of the pCR cases (FIG. 5A, FIG. 14). Z-scores derived from the outlier analysis for each of the data points (RNA, proteome and phosphoproteome) were used for single sample Gene Set Enrichment Analysis (ssGSEA). FIG. 5B highlights a subset of immune-centric and oncogenic signaling pathways that showed differential enrichment in the non-pCR cases. Consistent, significantly enriched pathway-level differences across replicate cores and multiple data types from a single patient was observed (FIG. 5B), confirming that microscaled proteogenomics data obtained from cores in a clinical setting yield reproducible results. Interestingly, distinct biological pathways showed differential enrichment in each of the individual non-pCR cases relative to the pCR class (FIG. 5B).

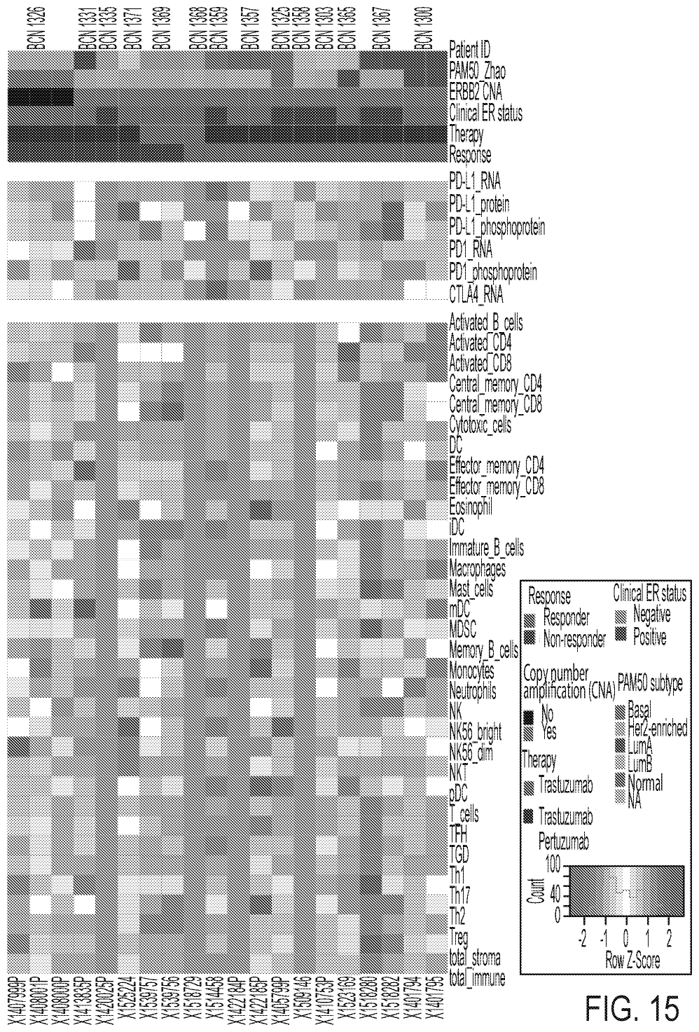

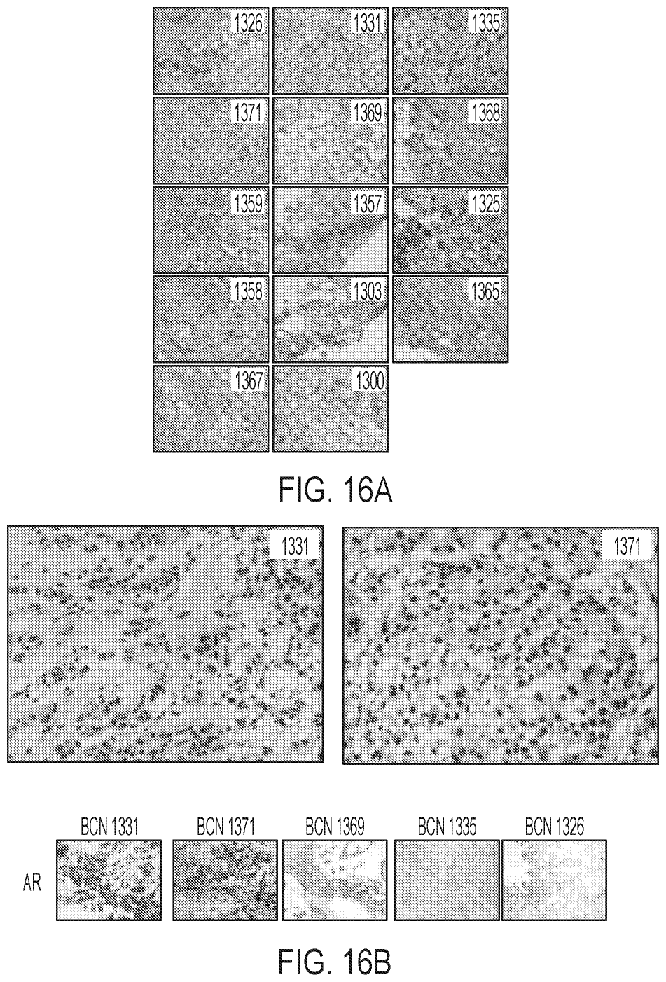

Of the complex patterns revealed by differential pathway analysis immune-related and interferon signaling pathways showed consistent upregulation across the data sets in samples from two of the three cases with lower expression of ERBB2, BCN1326 and BCN1331. In contrast, these pathways showed variable downregulation for the remaining non-pCR cases. To further explore these findings, the expression of T cell receptor (CD3 isoforms and CD247) and immune checkpoint (PD-L1, PD1, and CTLA4) genes were analyzed and immune profiles from the RNA-seq data using established tools were generated (Cibersort, ESTIMATE, and xCell). Examination of immune profile scores and of expression of T cell receptors and targetable immune checkpoint regulators supported the presence of an active immune response in BCN1326 relative to other samples (FIG. 5C, FIG. 15). Similarly, immune profile scores also indicated that BCN1331 had an activated immune microenvironment, and PD1 RNA expression was higher in this patient than in any other case (FIG. 5C). The five non-pCR cases were stained for the pan T-cell marker CD3 to validate these proteogenomic findings (FIG. 5D). Consistent with the active immune microenvironment (FIG. 5C), both BCN1326 and BCN1331 demonstrated tumor T-cell infiltration. In contrast, a predominant peri-tumoral or “immune-excluded” inflammatory reaction was observed in BCN1335 and a complete paucity of T-cells (immune-desert) was observed in the two resistant proteogenomically confirmed ERBB2+ cases, BCN1371 and BCN1369, consistent with the lack of immune signaling documented in FIG. 5B.

Other variable differential features in resistant cases included PI3K/AKT/mTOR and MAPK signaling, all of which represent potential therapeutic opportunities. ERBB2 pathway activation in BCN1331 is unexpected given the very low level of ERBB2 protein but could be explained by expression of EGFR/pEGFR (FIG. 3E). MYC targets were consistently upregulated at the protein and phosphoprotein levels in BCN1326 and BCN1335 and the androgen response pathway was upregulated at all levels in BCN1371. Consistent with the elevated AR signaling observed in BCN1371 (FIG. 5B), this tumor exhibited histologic features of an apocrine cancer with intensely eosinophilic cytoplasm and AR expression (FIG. 16 middle and lower panel). Interestingly, BCN1331 also expressed AR by IHC (FIG. 5D) without activation of an androgen response signature or apocrine features (FIG. 16), consistent, with the disconnect between AR expression and AR signaling in breast cancer noted by others²⁵. Also, for patient BCN1371, significant upregulation of PI3K signaling (FIG. 5B) was not seen despite PIK3CA mutation (E545K), consistent, with the disconnects between PIK3CA mutation and effects when signaling was assessed by reverse phase protein array (RPPA)^{18,26}

### Example 7

### ERBB2+ Patient Derived Xenograft (PDX) Models Provide a Setting to Explore Candidate Trastuzumab Resistance Mechanisms and Therapeutic Alternatives

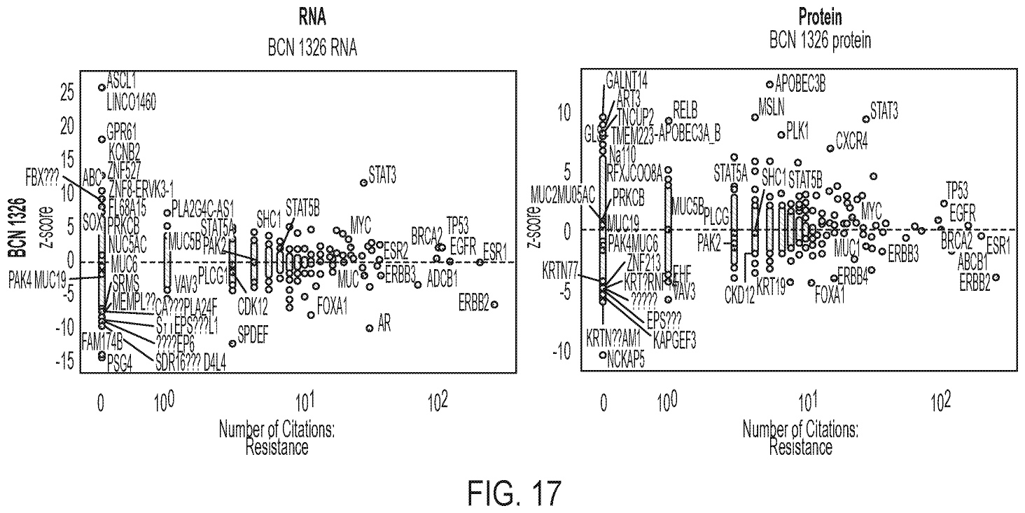

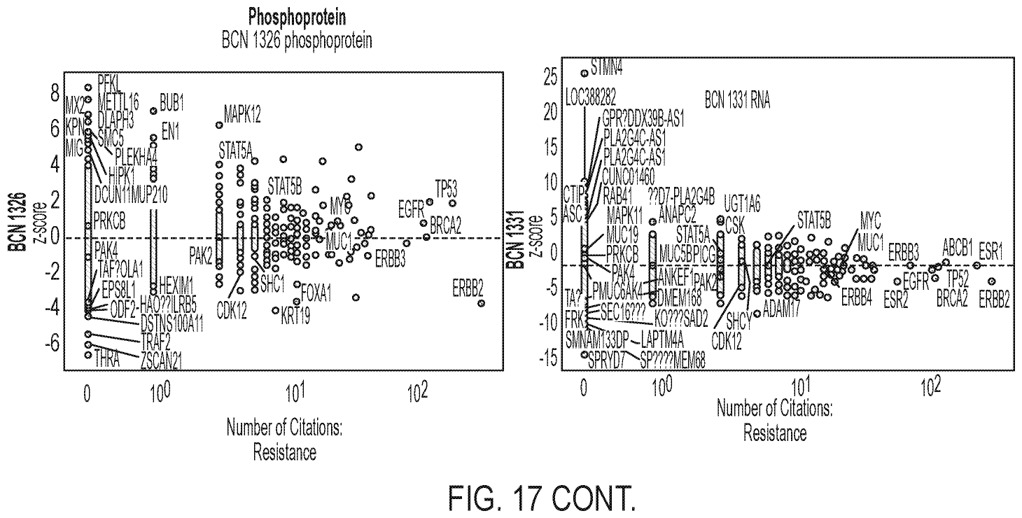

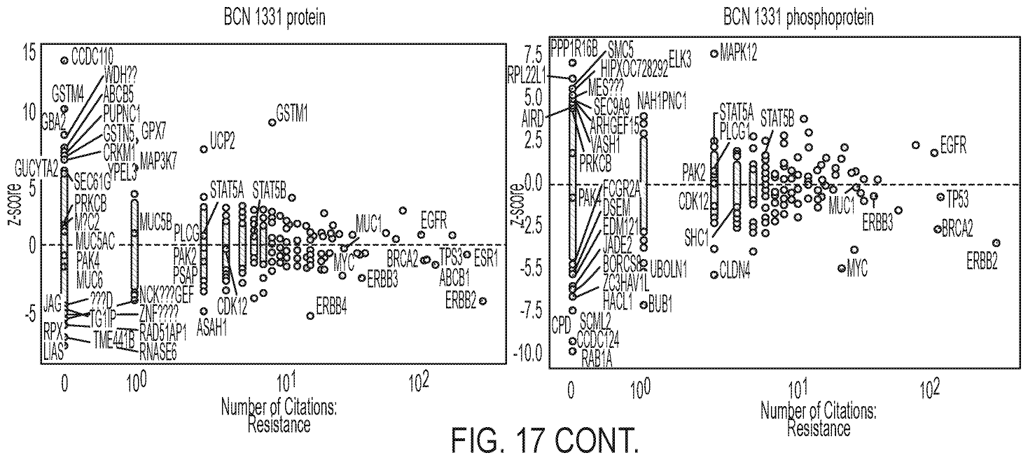

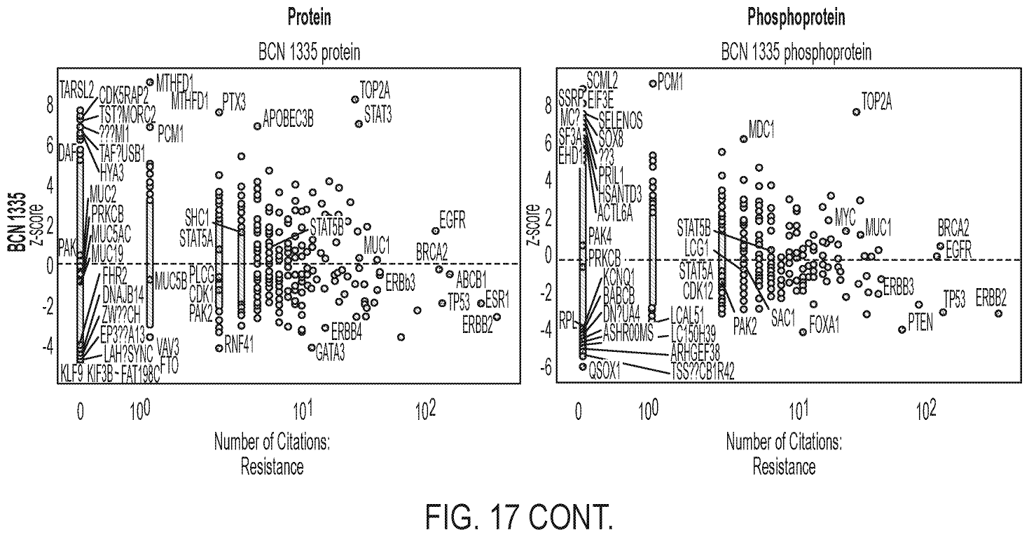

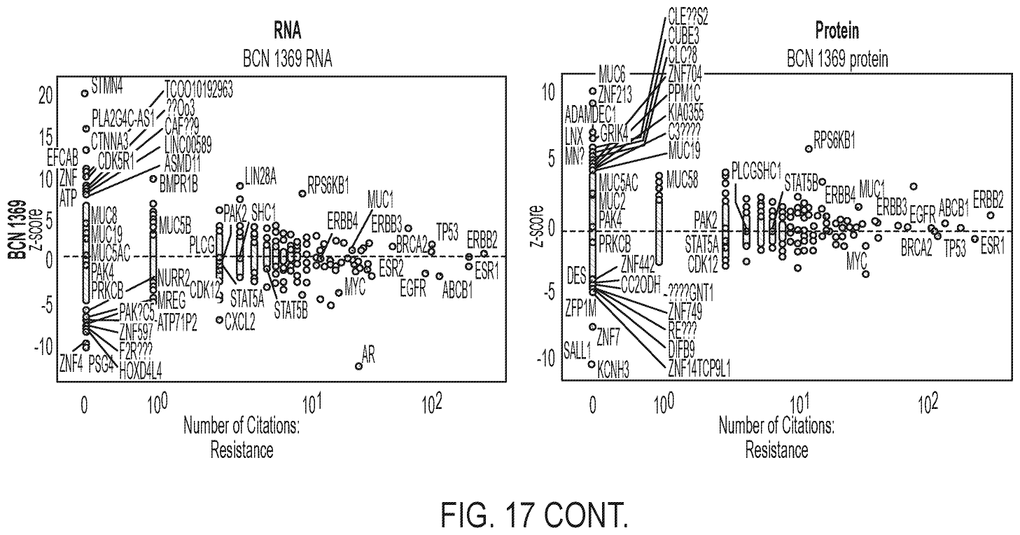

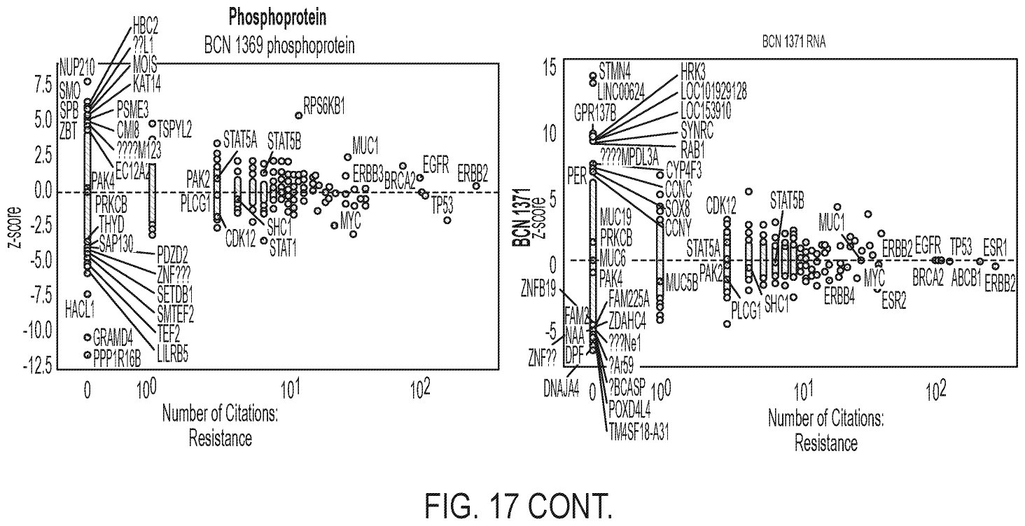

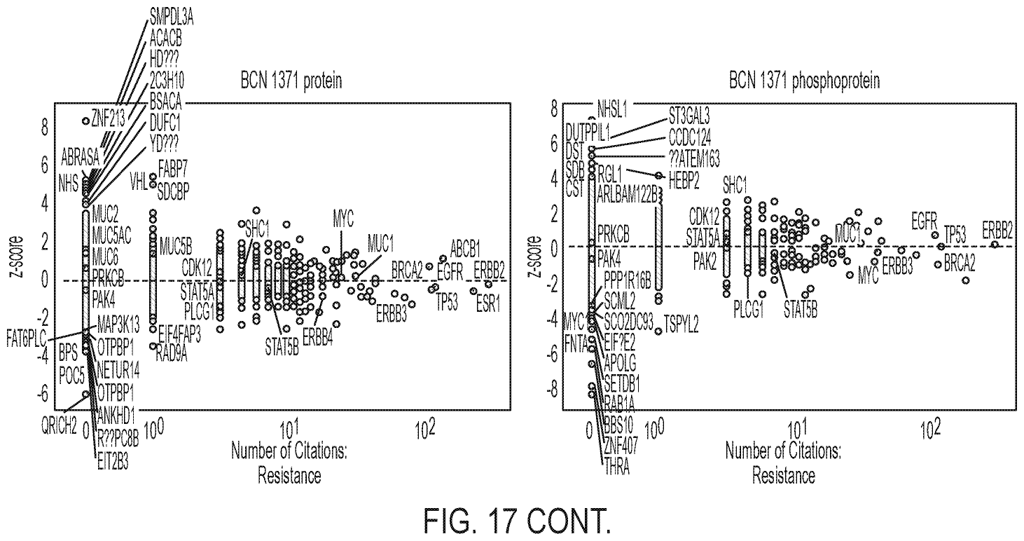

To further explore therapeutic resistance pathways in the proteogenomic data, association analyses between patient-centric RNA, protein and phosphoprotein outliers and the published literature were performed (FIG. 17). For each gene or protein, the terms “breast cancer” and “resist OR recur” were used to query the text from freely accessible article abstracts and keywords in the PubMed database to search for previously studied associations between the outlier genes and breast cancer resistance. Genes with highest filtered PubMed citation counts include well-studied proteins such as ESR1, BRCA1/2, TP53, EGFR, and AKT1 in addition to ERBB2. As expected, ERBB2 was among the most prominent negative protein and phosphoprotein outliers in BCN1326, BCN1331, and BCN1335 that were associated with the keyword “resistance” (FIG. 17). Furthermore, TOP2A also stands out as being strongly associated with “resistance” from other outliers with high protein and phosphoprotein levels in BCN1335, the psuedo-ERBB2+ patient for whom the amplified locus appears to be driving TOP2A rather than ERBB2 expression (FIG. 17; FIG. 3D). However, the most prominent proteomics outlier for patient BCN1369, MUC6, was not associated with citations containing the keyword “resistance” (FIG. 17). Nonetheless, multiple mucin family members were outliers with high protein expression specifically in this patient, two of which had citations associated with “resistance” (FIG. 17). The consistently high levels of mucin protein expression in patient BCN1369 are clearly discernible in the heatmap shown in FIG. 5E. This observation is notable because mucin expression has been proposed to mask ERBB2 epitopes and prevent trastuzumab binding, as shown previously in cell lines^{10,27,28}.

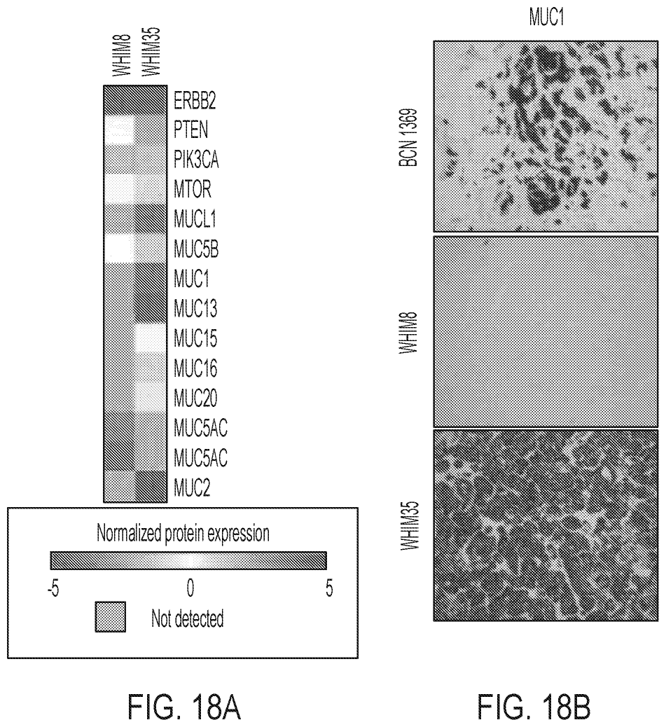

Since therapeutic hypotheses cannot be explored directly in non-pCR patients, the foregoing hypotheses about potential mechanisms of resistance are entirely speculative. To build an approach whereby potential resistance mechanisms could be explored experimentally, a published proteogenomic analysis was analyzed to determine of any of features of the resistant tumors in the DP1 study were phenocopied in ERBB2+ patient-derived xenografts (PDX)⁶. This analysis focused on two ERBB2+ PDX models, WHIM8 and WHIM35 both of which were shown to be responsive to lapatinib, a small molecular inhibitor against ERBB2, indicating they were true ERBB2+ cases⁶. Interestingly, WHIM35 has high expression of mucin proteins compared to WHIM8 (FIG. 18A) indicating this case was a phenocopy of BCN1369 (FIG. 18B). Consistent with cell line-based studies reported in the literature²⁹⁻³² that proposed that mucin expression might inhibit trastuzumab-mediated response, trastuzumab induced tumor regression in WHIM8 but not in WHM35 (FIG. 5F⁷). Drawing from the observation that BCN1369 also exhibited elevated PI3K-Akt-mTOR signaling (FIG. 5P), together with a recent report that showed mTOR mediated MUC1 induction in multiple breast cancer cell lines²⁹, these PDX models were additionally treated with the small molecule mTOR inhibitor everolimus. Everolimus in combination with ERB1B2-targeted therapy induced significant regression (FIG. 5F) in the otherwise trastuzumab-resistant WHM35 model, providing support for mucin overexpression as a “candidate” resistance mechanism that could potentially be overcome by treatment with a small molecule inhibitor of ERBB2 or downstream targeting of mTOR activity.

TABLE 2
Proteogenomic Features And Potential Mechanisms of Resistance
for Tumors from non-pCR Cases

| | RNAseq- | | | | | |
|---|---|---|---|---|---|---|
| | PAM50 | HER2 | | Differential | | |
| | Subtype | Amplicon | Drugg- | Pathways | TILS and Immune | |
| Case | and ER | Structure | able | (relative to | Microenvironment | Treatments |
| Number | status | by WES | Mutation | pCR cases) | Proteomics | Received |
| BCN | Basal | No copy | | MYC UP | Infiltrating TILs | Doc, CP, |
| 1326 | ER+ | gain (not | | Mitosis UP | PDL1 high | T, P |
| | | amplified) | | IFN | Phospho-PD1 high | |
| | | Low | | signaling | | |
| | | ERBB2 | | UP | | |
| BCN | HER2-E | Broad | | ERBB2/3 | Infiltrating TILs | Doc, CP, |
| 1331 | ER+ | lower- | | MAPK UP | Phospho-PD1 high | T, P |
| | | level copy | | PI3K UP | | |
| | | gain Low | | mTOR UP | | |
| | | ERBB2 | | TN | | |
| | | (pseudo- | | signaling | | |
| | | amplified?) | | UP | | |
| BCN | RNA | Amplicon | BRCA1, | MYC UP | Peri-tumoral TILs | Doc, CP, |
| 1335 | failure | driving | R1780 > | Mitosis UP | | T, P |
| | ER+ | TOP2A not | stop | | | |
| | | ERBB2 (pseudo- | VAF = | | | |
| | | amplified?) | 30% | | | |
| BCN | HER2-E | Amplified | PIK3CA | AR trans- | No TILs | Doc, CP, |
| 1371 | ER− | | E545K | cription UP | | T, P |
| | | | VAF = | ERBB2/3 | | |
| | | | 73.5% | DOWN | | |
| | | | | MAPK | | |
| | | | | DOWN | | |
| | | | | PI3K | | |
| | | | | DOWN | | |
| | | | | mTOR | | |
| | | | | DOWN | | |
| BCN | HER2-E | Amplified | | ERBB2/3 | No TILs | PAC, T |
| 1369 | ER− | | | UP | | |
| | | | | MAPK UP | | |
| | | | | PI3K UP | | |
| | | | | mTOR UP | | |
| | | | | MUCIN | | |
| | | | | expression | | |
| | | | | UP | | |

Doc = docetaxel;
CP = carboplatin;
T = trastuzumab;
P = pertuzumab;
PAC = paclitaxel

Table 2 summarizes examples of resistance mechanisms in the five non-pCR cases and proteogenomics driven alternative treatment options, including the one validated through PDX modeling.

### Example 8

### Significance of Certain Embodiments

Certain embodiments of the present disclosure concern at least one deep-scale multi-omics profiling of core needle biopsy material obtained in a clinical setting using the combined, integrative, tissue-sparing “BioText” approach described herein and, for example, applies this optimized microscaling methodology to a small cohort of breast cancer patients treated with chemotherapy and anti-ERBB2 therapy. Prior efforts at proteomic and phosphoproteomic analysis of core needle biopsies of tumor tissue using “one-shot” based data-independent analysis^{33,34} or off-line SCX fractionation combined with a super-SILAC approach quantified ˜2,000-5,000 proteins and ˜3800 phosphorylation sites per core. Importantly, these prior studies did not perform genomic analyses on the same set of cores. By contrast, workflows encompassed herein provide deep-scale genomic, proteomic and phosphoproteomic analysis, identifying more than 11,000 proteins and 25,000 phosphorylation sites in PDXs and, >17,000 in clinical cores for integrative multi-omics analyses. The alternating tissue sectioning approach provides exceptional control over sample quality, reduced sampling bias and ensured sample consistency across the multi-omics analysis. An optimized multiplexing protocol enabled the achievement of this depth from as little as 25 μgs of peptides per core, rendering the pipeline viable for material obtained attained from a typical 14 to 22-gauge clinical biopsy needle.

Illustrated herein is the utility of these microscaled methods by applying them to a breast cancer clinical study, a key component of which was the collection of on-treatment samples 24-48 hours after anti-ERBB2 therapy was initiated. This allowed an assessment of the immediate effects of inhibiting the ERBB2 pathway and potentially provides an early time point to determine whether a patient is likely to experience a pCR. Despite the small cohort size, a statistically significant downregulation of ERBB2 protein and phosphosite levels was detected and also a phosphosite signature for downstream mTOR targets in pCR patients. Of the 7 (out of 21 total ERBB2 sites identified) phosphosites from ERBB2 with complete data across the cohort, all showed downregulation to varying extents in the pCR cases. Of the 21 sites identified, only two have been characterized in detail in cell lines (see the PhosphoSitePlus® website). These are pY-1248, a known auto-activation site³⁵, and pT-701, which may serve as a negative feedback site³⁶, although their in-vivo roles are largely unexplored. The role of downregulation of ERBB2 phosphorylation in response to treatment is complicated by the observed downregulation of ERBB2 protein levels, but from a biomarker perspective these are secondary questions that do not negate the primary conclusion wherein a valid pharmacokinetic observation was made. Essentially all understanding in the art of the complex signaling properties of ERBB2 arise from experimental systems not from patients under anti-ERBB2 treatment. Most importantly, the ability to resolve complexity in this setting to assess inhibition of ERBB2 signaling is also revealed by downregulation of a signature of target sites for mTOR, a kinase activated downstream of ERBB2, specifically in pCR patients (FIG. 4C).

An initial proteogenomic focus on ERBB2 is readily justified given the immense biological variability within tumors designated ERBB2 “positive”. The testing guidelines are designed to offer as many patients anti-ERBB2 treatment as possible, even though it is recognized that this “catch all” approach includes a number of true-negative cases that are unlikely to benefit from these treatments³⁷. Though the motivation to employ this approach is understandable, this over-treatment strategy can be challenged. Besides the considerable cost to the healthcare system, patients with tumors mislabeled as ERBB2 positive may not receive treatments that are more appropriate for their true diagnosis. From the analyses herein, there is evidence for at least three classes of resistance mechanisms to ERBB2-directed therapeutics. Unequivocal false-positives are exemplified by case BCN1326. In retrospect, this case was initially diagnosed by FISH and was not protein over-expression positive when re-analyzed using standard IHC (IHC 1+). Analysis of three independent pretreatment samples in this case helps rule out heterogeneity as a likely cause of the misdiagnosis. The second class of misclassification is “pseudo-ERBB2 positivity”. As represented by cases BCN1331 and BCN1335, there was evidence for amplification of ERBB2, but multiple lines of proteogenomic evidence suggest that ERBB2 was not a strong driver including: a) low levels of ERBB2 protein and phosphoprotein compared to pCR cases; b) low expression from other genes within the minimal ERBB2 amplicon (STARD3, PDAP3 and GRB7); and c) a paucity of expression of dimerization partners ERBB3 and ERBB4. The successful validation of ERBB2 levels using single shot parallel reaction monitoring hints at an more efficient approach than the TMT multiplex assay that ultimately could form the basis of a clinical assay (FIG. 11). The third resistance class shows lack of pCR despite proteogenomic evidence for true ERBB2 positivity. Here there are proteogenomic clues for potential mechanisms of resistance to consider, such as the upregulation of mucin proteins, active androgen signaling or the lack of an antitumor immune response.

For BCN1335 the proteogenomic profile (both DNA and protein) suggests that TOP2A is a more likely driver, with higher amplification and protein expression than ERBB2. Here, ERBB2 was on the shoulder of the amplicon giving rise to the potential misdiagnosis. The treatment of ERBB2+ breast cancer has moved away from anthracyclines³⁸, but, perhaps in cases such as this, doxorubicin could be reconsidered 39. The comprehensive nature of the proteogenomic data allows efficient exploration of multiple causes for treatment failure at the level of pathway activity, illustrated by androgen receptor signaling in BCN1371 (FIG. 5B) and mucin expression in BCN1369 (FIG. 5E). For both examples, there is prior evidence for a causal role in resistance to trastuzumab but persistent controversy regarding the clinical relevance of these mechanisms^{28,40,41} This could be resolved in larger scale studies using the approaches.

The PDX experiments described herein are designed to illustrate how proteogenomic analysis can identify PDX that “phenocopy” potential resistance mechanisms observed clinical specimens so that therapeutic alternatives can be explored^{3, 6}. In-silico analysis of earlier published data rapidly identified a mucin-high (WHIM35) and a mucin-low (WHIM8) ERBB2+ pair of PDX tumors suitable for exploring alternative treatments for true ERBB2+ tumors that are mucin positive and trastuzumab-resistant. Pathway analyses of the mucin-positive clinical case BCN1369 indicated strong mTOR activity at the RNA, protein and phosphoprotein levels, suggesting that a therapeutic intervention with everolimus, an FDA-approved rapamycin-based mTOR inhibitor for ER+ advanced breast cancer⁴², could provide an effective treatment in the setting of mucin-driven resistance. Subsequent therapeutic modeling confirmed everolimus specifically increased trastuzumab efficacy in WHIM35 but not in the mucin-negative WHIM8 PDX, where trastuzumab alone was effective and everolimus may even be antagonistic. Presumably, lower molecular weight drugs, as opposed to monoclonal antibodies, can more readily diffuse into a mucin-positive tumor. Consistent with this conclusion, earlier data indicated that WHIM35 and WHIM8 are equally responsive to the small molecule ERBB2 inhibitor lapatinib⁶, and further pharmacodynamic analysis indicated that trastuzumab failed to suppress pERBB2 in the mucin-positive BCN1369 case (FIG. 4A lower panel). From a proteogenomic standpoint, mucin expression alone is likely not adequate to identify the correct population for a prospective clinical trial, as the establishment of true ERBB2+ status and a high mTOR signature may also be important.

Another important feature of the microscaled proteogenomic analysis presented herein is the ability to assess the immune microenvironment. This has become a critical aspect of breast cancer diagnostics with the approval of the PDL1 inhibitor atezolizumab in PDL1+ advanced TNBC, particularly since the diagnostics used to indicate efficacy (PDL1 IHC) can be fairly described as rudimentary⁴³. Cases BCN1326 and BCN1331, both potential ERBB2 protein negative cases, exhibited stronger immune signatures than true ERBB2+ cases and displayed proteomic evidence for PDL1, phospho-PD-L1, and phospho-PD1 expression. Post approval of a PDL1-monoclonal for TNBC treatment⁴³, a false or pseudo-false ERBB2+ misdiagnosis may matters because some of these cases could actually be PDL1+ TNBC and thus require a regimen that includes a PD1 or PDL1 monoclonal antibody. Conversely, both unresponsive true positives, BCN1371 and BCN1369, have reduced immune signatures and no TIL consistent with reports of poor long-term outcomes for immunologically cold ERBB2+ tumors 44.

While the microscaled proteogenomic methods were deployed here in the context of a clinical trial in breast cancer, they are patently extensible to any other solid tumor. Advancements in the art may reduce the time required for methods disclosed herein with automation of sample processing, use of faster instrumentation and orthogonal gas phase fraction such as FAIMS⁴⁵⁻⁴⁷. Furthermore, the methods as presented can be readily adapted for use as a diagnostic tool, for example by redirecting some of the denatured protein obtained using the BioTExt procedure to parallel reaction monitoring (PRM) assays developed for targets delineated in larger clinical discovery datasets, and, as illustrated for ERBB2 (FIG. 11).

In conclusion, the data provided herein demonstrates that microscaled proteogenomics increases precision in oncology treatment. Despite a small cohort size, a significant downregulation of potential markers of response upon treatment with ERBB2 inhibitors was observed in pCR patients and illustrated efficient exploration of resistance mechanisms in non-pCR patients. Besides confirming driver kinase status, proteogenomic methods are valuable for documenting that a pharmacokinetic response to drugging a driver kinase is present, before committing to a longer-term treatment regimen that might not be “on target”.

### Example 9

### Materials and Methods for General Embodiments

Patient-Derived Xenografts and Drug Treatment.

For PDX studies, all animal procedures were approved by the Institutional Animal Care and Use Committee at Baylor College of Medicine (Houston, Tex., USA) (protocol #AN-6934). 2-3 mm tumor pieces from PDX tumors were engrafted into cleared mammary fat pads of 3-4 weeks old SCID/bg mice (Envigo) and allowed to grow without exogenous estrogen supplementation until tumors reached 200-250 mm³. For head-to-head core and bulk comparison experiment, two non-adjacent cores were first obtained from the PDX models and immediately embedded in optimal cutting temperature medium and snap-frozen in liquid nitrogen. Following coring, tumors were surgically resected, and the tumor bulk was snap-frozen in liquid nitrogen. For treatment experiments, mice were randomized into 4 groups receiving i) vehicle or control ii) everolimus (5 mg/kg/BW/day in chow daily); iii) trastuzumab (30 mg/kg/BW/week; intraperitoneally) combination of trastuzumab and everolimus with n=15 mice/arm. Tumor volumes were measured by caliper every 3-4 days. For all animal experiments, tumor volumes were calculated by V=4/3×π×(length/2)²×(width/2). Baseline samples were collected on the day of randomization and treatment start date followed by sample collection at 1 week and 4 week post treatment. Animals were sacrificed when tumors reached 1500 mm³ or at the study end time-point.

DP1 Clinical Data

Following informed consent, patients diagnosed with either ERBB2− positive or Triple Negative breast cancer via diagnostic breast biopsy were enrolled in the National Surgical Adjuvant Breast and Bowel Project (NSABP) Biospecimen Discovery Project (DP) for ERBB2+ breast cancer. In accordance with consent, regular cancer care and optional additional 14-gauge needle biopsies preserved in optimal cutting temperature (OCT) fixative collected at diagnostic breast biopsy and 48 to 72 hours following paclitaxel and trastuzumab treatment along with blood samples collected and compacted to a frozen pellet before the start of standard treatment, up to 3 weeks after the first dose but before second dose, and at the time of surgery were sent to Washington University (St. Louis, Mo.) for research purposes.

Biopsy samples, blood samples, and medical information (including pathology reports about your breast cancer) were collected and labeled with a study number, which was a unique code assigned to samples and medical information. This unique code number which links a patient's name was separate from sample information. Sample information was given a separate BCN number (i.e. BCN “XXXX”) upon enrollment in the study. All subsequent sample derivatives were associated with their BCN number and has its own unique label ID.

Patients were able to withdraw samples without any penalty or loss of benefits entitled. However, in order to protect the anonymity of the databases, DNA sequences or other information that came from samples once entered into databases were not removed to prevent the risk of identification.

Biopsy Trifecta Extraction (BioTExt)

Embedding and sectioning: 14-gauge needle human biopsies were embedded in OCT fixative and stored at −80° C. Utilizing a cryostat maintained between −15 to −23° C., each biopsy was sectioned at 50 microns. Six (6) 50 micron curls were alternated amongst three (3) 1.5 mL microcentrifuge tubes assigned for denatured protein-DNA, native protein-DNA, or RNA extraction. At the start of sectioning and after an interval of six (6) curls were sectioned, a 5 micron curl was mounted on a slide for Hematoxylin and Eosin (H&E) staining and histopathological confirmation. This process was repeated until six (6) 50 micron curls were collected in all tubes per sample. The samples were then shipped from Washington University (St. Louis, Mo.) to Baylor College of Medicine (Houston, Tex.) for subsequent processing.

Histopathology confirmation: The tumor content percentages of each biopsy H&E slide (TC1, TC2, TC3, and TC4) were recorded and calculated to form a mean tumor content (avgTC). Those biopsies with an avgTC less than 50% were removed from further processing.

Immunohistochemistry (IHC): Tissue sections on charged glass slides were cut to 5 μm and deparaffinised in xylene and rehydrated via an ethanol step gradient. Peroxidase blocking, heat-induced antigen retrieval, and primary antibody conditions were performed per standard protocol under the following abbreviated conditions: ERBB2 (SP3, Neomarkers) 1:100, Tris pH 9.0; AR (441, sc-7305, Santa Cruz) 1:50, Tris pH 9.0; Muc1 (sc-7313, Santa Cruz) 1:150, Citrate pH 6.0; CD3 (polyclonal, A0452, Dako) 1:100, Tris pH 9.0. All primary antibodies were incubated at room temperature for 1 hour followed by standard chromogenic staining with the Envision Polymer-HRP anti-mouse/3,3′diaminobenzidine (DAB; Dako) process. Immunohistochemistry scoring was performed using established guidelines, when appropriate. All IHC results were evaluated against positive and negative controls.

DNA extraction: DNA was isolated via QIAamp DNA Mini Kit (Qiagen; 51306). DNA pellets were equilibrated to room temperature. 100 μL of Buffer ATL and then 20 μL of proteinase K was added to each sample and mixed by vortex. Samples were then incubated at 56° C. for 3 hours in a shaking heat block. Following incubation, samples were briefly centrifuged. 20 μL of RNase A (20 mg/mL) was added to each sample, pulse-vortexed for 15 seconds, and incubated for 2 minutes at room temperature. Samples were briefly centrifuged then pulse-vortexed for 15 seconds and incubated at 70° C. for 10 minutes. Following a brief centrifuge, 200 μL of Buffer AL was added, pulse-vortexed for 15 seconds, and incubated for an additional 70° C. for 10 minutes. Following another brief centrifugation, samples were carefully applied to a corresponding QIAamp Mini spin column placed in a collection tube without wetting the rim. The spin columns with sample were centrifuged at 6000×g for 1 min and then placed in a new collection tube while discarding the original filtrate. 500 μL of Buffer AW2 was added to spin columns without wetting the rim. Spin columns were centrifuged at maximum speed (20,000×g) for 3 minutes. Following centrifugation, the spin columns were placed in new collection tubes and once again centrifuged at maximum speed for 1 minute. Spin columns were then placed in new 1.5 mL micro-centrifuge tubes. 100 μL of Buffer AE was added to each spin column and incubated at room temperature for 5 minute while in a shaking heat block. The final DNA isolates were collected in their corresponding 1.5 mL tube following centrifugation at 6,000×g for 1 minute. DNA quality control was validated via Picogreen analysis.

RNA extraction: 1 mL of TRIzol Reagent (Thermo Fisher Scientific; 15596026) was added to each RNA-designated tube of cryo-sectioned curls and immediately inverted three times and transferred to a sonicator vial. Samples were individually sonicated in the S220 Ultrasonicator for 2 minutes at peak power: 100.0, duty factor: 10.0, cycles/burst: 500. All samples were then incubated for 5 minutes and transferred to a 1.5 mL microcentrifuge tube. Following addition of 200 μL of chloroform, each sample was incubated for 3 minutes and then centrifuged at 12,000×g for 15 minutes at 4° C. The supernatants were discarded. The pellet was air dried in the micro-centrifuged tube for 10 minutes. The pellet was re-suspended in 20 μL of RNase-free water and incubated at 56-60° C. in a heat block for 10-15 minutes. 10 μL of Buffer RDD and 2.5 μL of DNase I (Qiagen; 79254) was added to each sample. The sample volume was then brought up to 100 μL with RNase-free water and incubated at room temperature for 10 minutes. 350 μL of Buffer RLT was added and mixed well in each sample. Thereafter, 250 μL of 100% EtOH was mixed with each sample, and the mixture was quickly transferred to an RNeasy MinElute spin column (Qiagen; 74106) and placed in a 2 mL collection tube, which was then centrifuged at 12,000×g for 15 seconds. The flow through was discarded, and 500 μL of 80% EtOH was added to each spin column. The columns were centrifuged at 12,000×g for 2 minutes. The flow through was discarded and the column in placed in a new 2 mL collection tube. The samples were centrifuged at full speed for 5 minutes with the lid of the spin column open. Following centrifugation, the spin column was placed in a 1.5 mL micro-centrifuge tube and 14 μL of RNase-free water was directly added to the center of the spin column membrane. The spin column were centrifuged at max speed for 1 minute to elute the RNA. RNA quality control was validated via Picogreen analysis.

Denatured protein extraction: 1 mL of cold 70% ethanol (EtOH) was added to tubes assigned for denatured protein. Each tube was quickly pulse-vortexed for 30 seconds and briefly centrifuged at 20,000×g for 5 minutes at 4° C. 70% EtOH was carefully aspirated. 1 mL of cold NanoPure water was added, and the tube was quickly pulse-vortexed for 30 seconds and briefly centrifuged at 20,000×g for 5 minutes at 4° C. NanoPure water was carefully aspirated. 1 mL of cold 100% EtOH was added, and the tube was quickly pulse-vortexed for 30 seconds and briefly centrifuged at 20,000×g for 5 minutes at 4° C. 100% EtOH was carefully aspirated. 100 μL of denatured protein lysis buffer (8M urea, 75 mM NaCl, 1 mM EDTA, 50 mM Tris-Cl pH 8.0, 10 mM NaF, Phosphatase inhibitor cocktail 2 (Sigma; P5726), Phosphatase inhibitor cocktail 3 (Sigma; P0044), Aprotinin (Sigma; A6103), Leupeptin (Roche; 11017101001), PMSF (Sigma; 78830)) was added to each sample and transferred to a micro-sonicator vial. All samples were incubated on ice for 10 minutes. Following incubation, samples were individually sonicated in the S220 Ultrasonicator for 2 minutes at peak power: 100.0, duty factor: 10.0, cycles/burst: 500. Lysates were transferred to 1.5 mL labeled tubes and centrifuged at 4° C., maximum speed (20,000×g), for 30 minutes. Lysate supernatants were transferred to a new labeled tube. The remaining precipitated pellets were snap frozen for subsequent DNA isolation. Quality control was validated via mass spectrophotometer analysis.

Native protein extraction: 100 μL of native protein lysis buffer (50 mM HEPES pH 7.5, 150 mM NaCl, 0.5% Triton X-100, 1 mM EDTA, 1 mM EGTA, 10 mM NaF, 2.5 mM NaVO4, Protease inhibitor cocktail, Phosphatase inhibitor cocktail) was added to each native protein sample, which was then transferred to a micro-sonicator vial. Each lysate tube was assigned a trackable Mass Spectrometer label. Lysate concentration was measured via Bradford reagent in which 800 μL of deionized water was added to 10 μL of each sample. 200 μL of Bradford reagent was then added to each deionized water plus lysate aliquot. Each sample was inverted and transferred to assigned cuvettes. Lysates were measured via a spectrophotometer with a corresponding blank sample. The spectrophotometer reading was divided by 10 to compensate for the dilution.

Genomic Data Generation and Analysis

DNA Sample QC: DNA was PicoGreen quantified. Samples that met the minimum PicoGreen quantified input requirements (>300 ng DNA, preferred concentration 10 ng/ul) proceeded into the Somatic Whole Exome workflow.

Whole Exome Library Construction: DNA was processed for Somatic Whole Exome Sequencing. This process included library preparation, hybrid capture, sequencing with 76 bp paired-end reads, sample identification QC check, and product-utilized ligation-based library preparation followed by hybrid capture with the Illumina Rapid Capture Exome enrichment kit with 38 Mb target territory.

Fluidigm Fingerprint Check: By genotyping a panel of highly polymorphic SNPs (including SNPs on chromosomes X and Y), a unique genetic ‘fingerprint’ was generated for each sample. These genotypes are stored in the sample tracking database and compared automatically to genotypes from the production pipeline to ensure the integrity of sample tracking.

Exome Sequence Generation: All libraries were sequenced to attempt to meet a goal of 85% of targets covered at greater than 50× coverage (+/−5%) for tumor samples utilizing the Laboratory Picard bioinformatics pipeline. All sequencing was performed by the Laboratory on Illumina instruments with 76 base pair, paired-end sequencing. The Laboratory Picard pipeline aggregated all data from a particular sample into a single BAM file that included all reads, all bases from all reads, and original/vendor-assigned quality scores.

Identification of mutations and copy number alterations from whole exome sequencing: Identification of mutations from whole exome sequencing: VarScan2 was used to identify germline mutations (SNPs and INDELs) from the germline BAM files and somatic mutations by comparing the tumor BAM file to the germline BAM file for each patient. Annovar was then used to separately annotate SNP and INDEL vcf files from VarScan for germline and somatic mutations from each patient. Mutations with “non-synonymous SNVs”, “stopgain”, “stoploss”, and “splicing” annotations that affect the protein coding sequences of genes were extracted from the resulting SNP multianno files and combined into a single text file for all patients. Similarly, INDELs annotated as occurring in the exons of genes were extracted from each INDEL multianno file and combined into a single file.

Somatic Copy Number Alteration (SCNA) Analysis: the R Package CopywriteR (version 1.18.0⁴⁸) was used. For the analyses described in the manuscript, non-0 RSEM data for each sample was upper-quartile normalized, and genes with 0 read counts across all samples were removed (0 reads treated as NA). ESTIMATE, Cibersort, and xCell were used to infer stroma content (ESTIMATE and xCell), immune infiltration (all), and tumor immune cell profiles (Cibersort and xCell) using upper quartile normalized RSEM data, and log 2 transformed upper quartile normalized RSEM data was used for the outlier and LIMMA analyses described below⁴⁹⁻⁵³.

Proteomic Data Generation and Analysis

Experimental design: For the CPTAC workflow, the 4 PDX models were analyzed in process replicates (8 TMT channels) along with 2 common reference (CR) samples in a TMT ten-plex format. The first common reference (CR1) was constructed from equal proportions of peptides derived from the 4 cryopulverized PDX bulk tumors. The second common reference (CR2) had been used in a prior proteogenomic breast cancer PDX study that included these four models⁶. For the MiProt workflow, 8 individual cores comprising 2 cores per model (8 TMT channels) were analyzed along with 2 common references in TMT ten-plex format. The first CR (CR3) was composed of equal proportions of peptides from the 8 cores, while the second CR was an aliquot of the bulk CR (CR1), described above. Protein and phosphopeptide expression were reported as the log ratio of each sample's TMT intensity to the intensity of an internal common reference included in each plex, either CR1 for the CPTAC workflow or CR3 for the MiProt workflow. For analyzing patient derived core needle biopsies, TMT-eleven-plex format was used where first 9 channels contained peptides form 9 core needle biopsies and the last two channels (131N, 131C) contained two different CRs. Channel 131N contained CR4 that was constructed from equal proportion of peptides from all the 14 patients. Channel 131C contained CR5 that has been previously used to characterize a large cohort of breast cancer subtypes (see the data portal for the National Cancer Institute's Clinical Proteomic Tumor Analysis Consortium (CPTAC), accession number S039). For this manuscript, all ratios were calculated relative to CR4. For both PDX and clinical core analyses, samples within a TMT11 plex were randomized to reduce batch effects.

Sample preparation: Protein lysates in 8M Urea were treated with 1 mM DTT for 45 minutes followed by 2 mM iodoacetamide (IAA) for an additional 45 minutes. 8M Urea was diluted to a final concentration of 2M with 50 mM Tris-HCL pH 8.5. Protein lysates were incubated with endopeptidase LysC (Promega) at a concentration of 1:50 (ug of LysC to ug of Proteins) for 2 hours followed by overnight incubation with Trypsin (Promega) at a concentration of 1:30 (ug of Trypsin to ug of Proteins). Both enzymatic digestions were performed at room-temperature. Following protein digestion, peptides were acidified to a final concentration of 1% Formic acid followed by purification using 50 mg Sep-Pak cartridge (Waters). Peptides were eluted off the Sep-Pak cartridge with 50% acetonitrile and 0.1% formic acid. Peptide concentration was measured using 280 absorbance using a Nanodrop (Thermo Scientific). For qualitative assessment, 0.5 ug peptides were run on a nLC1200 coupled to Q-Exactive+LC-MS setup (Thermo Scientific). Eluted peptides were snap-frozen and dried using a speed-vac apparatus.

For CPTAC workflow, a total of 300 ug peptides were labeled with 800 ug TMT reagent as described previosly⁸. For the MiProt workflow a total of 25 ug peptides in 100 ul of 50 mM Hepes pH8.5 was labeled with 200 ug TMT reagent (8-fold excess). TMT and peptide mixture was incubated at room-temperature for 1 hour. Prior to the quenching of excess TMT reagent, a total of 1 ul per sample was stage-tipped onto a C18 disc (EMPORE C18) and a total of 0.5 ug of peptides was run on a 30 minutes gradient to assess TMT labeling efficiency. In addition, 2 ul from each sample were pooled together and desalted. A total of 0.5 ug peptides were run on a 110 minutes gradient to assess mixing ratios. Partial TMT labeling was allowed to be over 99%, fully TMT labeling to be over 94% and mixing ratios to be within +/−15% compared to the common internal reference (CR), which was 131N for TMT10-plex setup and 131C for TMT11-plex setup. Excess TMT reagent was quenched using 6 ul of 5% Hydroxylamine (Sigma) followed by a 15 minutes incubation. All the samples within a plex were mixed based on the mixing ratios to achieve equal amounts for all channels. Peptides were purified using a 100 mg Sep-Pak cartidge (Waters) and dried down using a speed-vac apparatus.

Basic reverse fractionation and Phospho-enrichment: For basic phase reverse (bRP) fractionation, ˜250 ug of peptides were dissolved in 500 ul of 5 mM ammonium formate and 5% acetonitrile. An offline Agilent 1260 LC coupled to 30 cm and 2.1 diameter column running at a flow-rate of 200 ul per minute was used for bRP fractionation. Peptides were fractionated into 72 fractions and finally concatenated into 24 fractions. A total of 2 ug peptides per fraction was transferred into the mass-spectrometer vial for whole proteome analysis, but only 0.5 ug per fraction was injected for whole proteome analysis. The 24 fractions were further concatenated (by pooling of every 6th fraction) into 4 fractions (˜62 ug peptides per fraction) for phosphopeptide enrichment.

CPTAC workflow has been described before². In brief, for the CPTAC workflow, 3000 ug of peptides were dissolved in 1000 ul of 5 mM ammonium formate and 5% acetonitrile. Offline fractionation was performed as described above using a 30 cm and 4.6 diameter column. A total of 72 fractions were concatenated into a total of 24 fractions and 0.5 ug peptides per fraction were analyzed for whole cell proteomics. The 24 fractions were further concatenated into a total of 12 fractions (by pooling of every 2nd fraction yielding ˜250 ug per fraction) for IMAC based phosphopeptide enrichment.

Phosphopeptide enrichment was done using Fe3+ immobilized metal affinity chromatography (IMAC). For this, Ni-NTA (Qiagen) beads were washed three times with HPLC grade water followed by incubation with 100 mM EDTA (Sigma) for 30 minutes to strip Ni²⁺ off the beads. The beads were washed 3 times with HPLC grade water followed by incubation with FeCl₃ (Sigma) for 45 minutes. Beads were again washed with HPLC grade water followed by resuspension of Fe₃⁺ loaded agarose beads with resuspension buffer containing methanol, acetonitrile and 0.01% acetic acid at 1:1:1 ratio. For both CPTAC and MiProt workflows, dried down peptides were resuspended to a final volume of 500 ul, with 50% acetonitrile and 0.1% trifluoroacetic acid (TFA) and supplemented with 97% acetonitrile and 0.1% TFA to a final concentration of 80% acetonitrile and 0.1% TFA. A total of 20 ul of 50% slurry was used per fraction for phosphopeptide enrichment. IMAC beads and peptides were incubated at room temperature for 30 minutes on a tumble-top rotator. Beads were spun down and resuspended with 200 ul of 80% acetonitrile and 0.1% TFA and transferred directly onto a conditioned C18 stage-tips. Phosphopeptides were eluted off the beads using 500 mM K₂HPO₄, pH 7 buffer onto C18 stage-tip, washed with 1% formic acid and finally eluted into a mass spectrometer LC vial using 50% acetonitrile and 0.1% FA.

Data acquisition: A Proxeon nLC-1200 coupled to Thermo Lumos instrumentation was used for proteome and phosphoproteome data acquisition. Peptides were run on a 110 minute gradients with 86 minutes of effective gradient (6 to 30% buffer B containing 90% ACN and 0.1% FA). For phosphoproteomics analysis of cores, a second injection was performed and analyzed over a 145 minute gradient with 120 minutes of effective gradient (6 to 30% buffer B containing 90% ACN and 0.1% FA). The acquisition parameters are as follows, MS1: resolution—60,000, MS1 injection time: 50 seconds, MS2: resolution: 50,000, MS2 injection time: 110 seconds, AGC 5E4. Data acquisition was performed with a cycle time of 2 seconds.

Proteomics data processing and normalization: Raw files were searched against the human (clinical samples) or human and mouse (PDX samples) RefSeq protein databases complemented with 553 small-open reading frames (smORFs) and common contaminants(Human:0 RefSeq.20111003_Human_ucsc_hg38_cpdb_mito_259contamsnr_553smORFS), (Human and Mouse: RefSeq.20160914_Human_Mouse_ucsc_hg19_mm10_customProDBnr_mito_150contams) using Spectrum Mill suite vB.06.01.202 (Broad Institute and Agilent Technologies) as previously described in detail⁶. For TMT quantification, the ‘Full, Lys only’option that requires lysine to be fully labeled while allowing under-labeling of peptides N-termini was used. Carbamidomethylation of cysteines was set as a fixed modification, and N-terminal protein acetylation, oxidation of methionine (Met-ox), de-amidation of asparagine, and cyclization of peptide N-terminal glutamine and carbamidomethylated cysteine to pyroglutamic acid (pyroGlu) and pyro-carbamidomethyl cysteine were set as variable modifications. For phosphoproteome analysis, phosphorylation of serine, threonine, and tyrosine were allowed as additional variable modifications, while de-amidation of asparagine was disabled. Trypsin Allow P was specified as the proteolytic enzyme with up to 4 missed cleavage sites allowed. For proteome analysis, the allowed precursor mass shift range was −18 to 64 Da to allow for pyroGlu and up to 4 Met-ox per peptide. For phosphoproteome analysis the range was expanded to −18 to 272 Da, to allow for up to 3 phosphorylations and 2 Met-ox per peptide. Precursor and product mass tolerances were set to ±20 ppm and peptide FDR to 1% employing a target-decoy approach using reversed protein sequences⁴². For PDX analyses, the subgroup-specific (SGS) option in Spectrum Mill was enabled as previously described⁶. This allowed us to better dissect proteins of human and mouse origin. If specific evidence for BOTH human and mouse peptides from an orthologous protein were observed, then peptides that cannot distinguish the two (shared) were ignored. However, the peptides shared between species were retained if there was specific evidence for only one of the species, thus yielding a protein group with a single subgroup attributed to only the single species consistent with the specific peptides.

For generation of protein and phosphopeptide ratios, reporter ion signals were corrected for isotope impurities and relative abundances of proteins, and phosphorylation sites were determined using the median of TMT reporter ion intensity ratios from all PSMs matching to the protein or phosphorylation site. PSMs lacking a TMT label, having a precursor ion purity <50%, or having a negative delta forward-reverse score (half of all false-positive identifications) were excluded. To normalize quantitative data across TMT10/11plex experiments, TMT intensities were divided by the specified common reference for each phosphosite and protein. Log 2 TMT rations were further normalized by median centering and median absolute deviation scaling.

Parallel Reaction Monitoring (PRM): Two unique peptides for ERBB2 protein (VLQGLPR (SEQ ID NO:39) and GLQSLPTHDPSPLQR) were used for PRM analysis. Peptides used for proteome analysis were analyzed by Orbitrap Fusion Lumos mass spectrometer coupled with EASY-nLCTM 1200 system (Thermo Fisher Scientific) for PRM. 1 ug of peptides was loaded to a trap column (150 m×2 cm, particle size 1.9 m) with a max pressure of 280 bar using Solvent A (0.1% formic acid in water), then separated on a silica microcolumn (150 m×5 cm, particle size, 1.9 m) with a gradient of 4-28% mobile phase B (90% acetonitrile and 0.1% formic acid) at a flow rate of 750 nl/min for 75 min. Both data-dependent acquisition (DDA) and PRM mode were used in parallel. For DDA scan, a precursor scan was performed in the Orbitrap by scanning m/z 300-1200 with a resolution of 120,000 at 200 m/z. The most 20 intense ions were isolated by Quadrupole with a 2 m/z window and fragmented by higher energy collisional dissociation (HCD) with normalized collision energy of 32% and detected by ion trap with rapid scan rate. Automatic gain control targets were 5×105 ions with a maximum injection time of 50 ms for precursor scans and 104 with a maximum injection time of 50 ms for MS2 scans. Dynamic exclusion time was 20 seconds (±7 ppm). For PRM scan, pre-selected peptides were isolated by quadrupole with a 0.7 m/z window followed by HCD with normalized collision energy of 32% and product ions (MS2) were scanned by Orbitrap with a resolution of 30,000 at 200 m/z. Scan windows were set to 4 min for each peptide. For relative quantification, the raw spectrum file was crunched to .mgf format by Proteome Discoverer™ 2.0 software (Thermo Fisher Scientific) and then imported to Skyline with raw data file. Each result was validated by deleting non-identified spectrum and adjusting the AUC range. Finally, the sum of the area of at least six strongest product ions for each peptide was used for the result.

Bioinformatic Data Analysis

Outlier analysis: The data for each gene or protein from the set of baseline samples from the patients that showed pathological complete response was used to establish a normal distribution for that gene/protein. For each gene, a Z-score for each baseline sample from the non-pCR case was calculated by determining the number of standard deviations the expression value in the non-responder deviated from the mean of this distribution. Genes/proteins with low variance (variance <1.5) and that did not have a normal distribution, by Shapiro-Wilk test, in the patients showing pathological complete response were removed prior to subsequent analysis. For phosphoprotein level outlier analysis, the mean level of all phosphosites for each protein was evaluated. Pathway analysis using single sample Gene Set Enrichment Analysis (ssGSEA) and the MSigDb gene sets was carried out on the Z-scores for each dataset in each non-pCR sample using the parameters described below.

limma differential analysis of response to treatment in pCR and non-pCR patients: The limma R package was used to analyze the set of patients with both on-treatment and pre-treatment cores in order to compare on-treatment vs. pre-treatment expression in pCR and non-pCR patients separately in each dataset (RNA, protein, phosphoprotein (mean phosphosite level for each protein), and phosphosite datasets) and to compare on-treatment vs. pre-treatment changes in expression in pCR patients to non-pCR patients. Samples from BCN1368 and BCN1369 were excluded from this analysis because of they did not receive the full treatment regimen (didn't get pertuzumab). Phosphosite level data for this analysis was first processed by taking the mean of all peptides containing each fully localized site as determined by Spectrum Mill. For this analysis, duplicate cores for a given patient were included but the limma duplicateCorrelation function was used to derive a consensus for each patient for the differential analysis. Each gene (or site) in each dataset was fitted to a linear model with coefficients for each group (on-treatment pCR, pre-treatment pCR, on-treatment non-pCR, and pre-treatment non-PCR) and each plex (to account for batch effects), and moderated T-tests for each comparison were carried out by limma using the residual variances estimated from the linear models. PTM-SEA was applied to signed, log 10 transformed p-values from this analysis using the parameters described below.

Gene-Set Enrichment Analysis and PTM-Signature Enrichment Analyses

Pathway analysis was performed using single sample Gene Set Enrichment Analysis (ssGSEA) and post-translational modification signature enrichment analysis (PTM-SEA). Protein and phosphosite measurements of technical replicates were combined by taking the average across replicates before subsequent analysis. Pathway level comparisons of bulk and core material were based on signed, log 10-transformed p-values derived from a moderated two-sample T-test using the limma R-package comparing luminal and basal tumors separately for bulk and core samples. For proteome data the two-sample moderated T-test was first applied for each protein and resulting transformed p-values (see above) were collapsed to gene-centric level for ssGSEA by retaining the most significant p-value per gene symbol. Phosphosite-level data were subjected to limma-analysis to derive transformed p-values (see above) for each phosphorylation site. Sequence windows flanking the phosphorylation site by 7 amino acids in both directions were used as unique site identifier. For PTM-SEA, only fully localized phosphorylation sites as determined by Spectrum Mill software were taking into consideration. Phosphorylation sites on multiply phosphorylated peptides were resolved as described previously¹⁴. An in-house Python script was used to drive queries using NCBI-s E-utilities, and resulting freely available information (title, abstract, keywords) were saved to a local SQL database. For each publication, a case-insensitive text search for “resist” OR “recur” AND “breast cancer” was performed, with positive hits retained and tallied for each gene. Publications with over 100 different gene associations were excluded to avoid false positives from high-throughput studies.

### Example 10

### Proteogenomic Classification of HER2 Status in Breast Cancer Patients

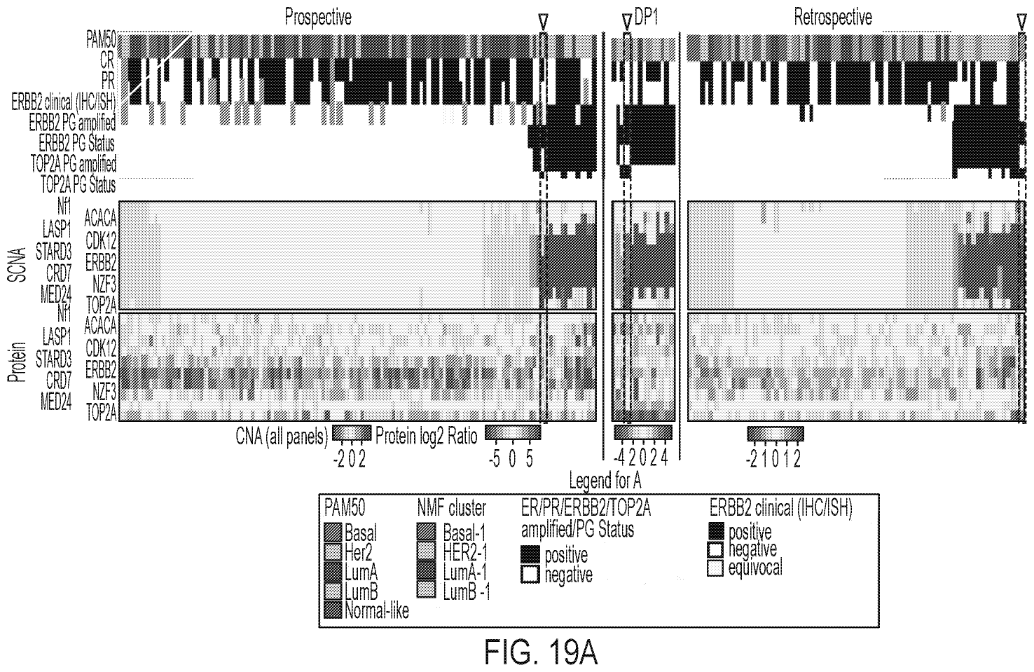

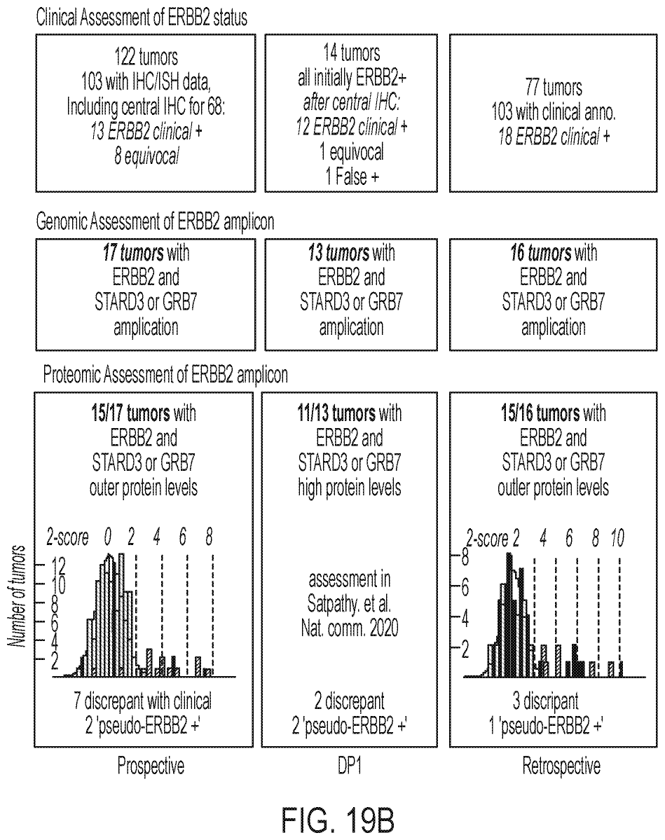

The present example concerns proteogenomic classification of HER2 status in breast cancer patients. Shown in FIG. 19 are examples of ERBB2 proteogenomic positive (PG+) tumors, where the ERBB2 gene is amplified and over-expressed at the protein level along with adjacent genes in the locus (i.e., STARD3 and GRB7), in two additional larger cancer cohorts: a prospective study utilizing data for the present disclosure and a retrospective study that was published in 2016 (Mertins et al., Nature. 2016 May 25; 534(7605): 55-62). A Discovery protocol 1 (DP1); NCT01850628 study referred to elsewhere herein is also included as a point of reference.

FIG. 19A shows examples of clinical false positives (ERBB2 clinical (IHC/ISH)) that were found to be ERBB2 PG− when evaluating the genomic and proteomic data; these are the patients (columns) where the box is black (or yellow if less stringent criteria are applied) for the ERBB2 clinical row but white for the ERBB2 PG Status row. It also shows examples of ERBB2 psuedo-PG+ tumors, where the gene and its adjacent genes are amplified without over-expressing the proteins; these are the samples that are black in the ERBB2 amplified row but white in the ERBB2 PG Status row. Also shown are examples of ERBB2 psuedo-PG+ tumors where the TOP2A gene is also amplified and is over-expressed despite lack of ERBB2 expression, and these are marked (red arrows and blue boxes).

FIG. 19B shows a breakdown of the samples for each of the three classifications mentioned above, ERBB2 clinical status, ERBB2 amplification status, and ERBB2 PG Status, in each cohort. It demonstrates how protein over-expression (proteomic assessment) was defined in the added studies. To clarify, DP1 consisted of mostly HER2/ERBB2+ patients, and the inventors looked for outliers with lower expression of ERBB2 and neighboring proteins and classified them as ERBB2 PG− for the PD1 study. However, for the other two studies referred to above, most of the patients were ERBB2/HER2−, and the inventors looked at outliers with higher expression of ERBB2 and neighboring genes to identify the PG+ samples.

### REFERENCES

1. Archer, T. C., et al. Proteomics, Post-translational Modifications, and Integrative Analyses Reveal Molecular Heterogeneity within Medulloblastoma Subgroups. Cancer Cell 34, 396-410.e398 (2018).
2. Mertins, P., et al. Proteogenomics connects somatic mutations to signalling in breast cancer. Nature 534, 55-62 (2016).
3. Mundt, F., et al. Mass Spectrometry-Based Proteomics Reveals Potential Roles of NEK9 and MAP2K4 in Resistance to PI3K Inhibition in Triple-Negative Breast Cancers. Cancer Res. 78, 2732-2746 (2018).
4. Zhang, B., et al. Proteogenomic characterization of human colon and rectal cancer. Nature 513, 382-387 (2014).
5. Vasaikar, S., et al. Proteogenomic Analysis of Human Colon Cancer Reveals New Therapeutic Opportunities. Cell 177, 1035-1049 e1019 (2019).
6. Huang, K.-L., et al. Proteogenomic integration reveals therapeutic targets in breast cancer xenografts. Nat. Commun. 8, 14864 (2017).
7. Matsunuma, R., et al. DPYSL3 modulates mitosis, migration, and epithelial-to-mesenchymal transition in claudin-low breast cancer. Proc. Natl. Acad. Sci. U.S.A 115, E11978-E11987 (2018).
8. Mertins, P., et al. Reproducible workflow for multiplexed deep-scale proteome and phosphoproteome analysis of tumor tissues by liquid chromatography-mass spectrometry. Nat. Protoc. 13, 1632-1661 (2018).
9. Slamon, D. J., et al. Human breast cancer: correlation of relapse and survival with amplification of the HER-2/neu oncogene. Science 235, 177-182 (1987).
10. Vu, T. & Claret, F. X. Trastuzumab: updated mechanisms of action and resistance in breast cancer. Front. Oncol. 2, 62 (2012).
11. Li, S., et al. Endocrine-therapy-resistant ESR1 variants revealed by genomic characterization of breast-cancer-derived xenografts. Cell Rep. 4, 1116-1130 (2013).
12. Thompson, A., et al. Tandem mass tags: a novel quantification strategy for comparative analysis of complex protein mixtures by MS/MS. Anal. Chem. 75, 1895-1904 (2003).
13. Zecha, J., et al. TMT labeling for the masses: A robust and cost-efficient, in-solution labeling approach. Mol Cell Proteomics (2019).
14. Krug, K., et al. A curated resource for phosphosite-specific signature analysis. Mol. Cell. Proteomics (2018).
15. Subramanian, A., et al. Gene set enrichment analysis: a knowledge-based approach for interpreting genome-wide expression profiles. Proc. Natl. Acad. Sci. U.S.A 102, 15545-15550 (2005).
16. McShane, L. M., et al. REporting recommendations for tumour MARKer prognostic studies (REMARK). European Journal of Cancer 41, 1690-1696 (2005).
17. Zhang, H., et al. Integrated Proteogenomic Characterization of Human High-Grade Serous Ovarian Cancer. Cell 166, 755-765 (2016).
18. Cancer Genome Atlas, N. Comprehensive molecular portraits of human breast tumours. Nature 490, 61-70 (2012).
19. Ferrari, A., et al. A whole-genome sequence and transcriptome perspective on HER2-positive breast cancers. Nat Commun 7, 12222 (2016).
20. Wang, J., et al. Proteome Profiling Outperforms Transcriptome Profiling for Coexpression Based Gene Function Prediction. Mol. Cell. Proteomics 16, 121-134 (2017).
21. Roskoski, R., Jr. The ErbB/HER family of protein-tyrosine kinases and cancer. Pharmacol Res 79, 34-74 (2014).
22. Hsu, J. L. & Hung, M. C. The role of HER2, EGFR, and other receptor tyrosine kinases in breast cancer. Cancer Metastasis Rev 35, 575-588 (2016).
23. Ritchie, M. E., et al. limma powers differential expression analyses for RNA-sequencing and microarray studies. Nucleic Acids Res 43, e47 (2015).
24. Dankort, D., et al. Grb2 and She adapter proteins play distinct roles in Neu (ErbB-2)-induced mammary tumorigenesis: implications for human breast cancer. Mol. Cell. Biol. 21, 1540-1551 (2001).
25. D'Arcy, C. & Quinn, C. M. Apocrine lesions of the breast: part 2 of a two-part review. Invasive apocrine carcinoma, the molecular apocrine signature and utility of immunohistochemistry in the diagnosis of apocrine lesions of the breast. J Clin Pathol 72, 7-11 (2019).
26. Stemke-Hale, K., et al. An integrative genomic and proteomic analysis of PIK3CA, PTEN, and AKT mutations in breast cancer. Cancer Res 68, 6084-6091 (2008).
27. Farahmand, L., Merikhian, P., Jalili, N., Darvishi, B. & Majidzadeh-A, K. Significant Role of MUC1 in Development of Resistance to Currently Existing Anti-cancer Therapeutic Agents. Curr. Cancer Drug Targets 18, 737-748 (2018).
28. Namba, M., et al. Anti-KL-6/MUC1 monoclonal antibody reverses resistance to trastuzumab-mediated antibody-dependent cell-mediated cytotoxicity by capping MUC1. Cancer Lett. 442, 31-39 (2019).
29. de Boer, H. R., et al. Quantitative proteomics analysis identifies MUC1 as an effect sensor of EGFR inhibition. Oncogene 38, 1477-1488 (2019).
30. Carraway, K. L., et al. Muc4/sialomucin complex in the mammary gland and breast cancer. J Mammary Gland Biol Neoplasia 6, 323-337 (2001).
31. Nagy, P., et al. Decreased accessibility and lack of activation of ErbB2 in JIMT-1, a herceptin-resistant, MUC4-expressing breast cancer cell line. Cancer Res 65, 473-482 (2005).
32. Mercogliano, M. F., et al. TNFalpha-Induced Mucin 4 Expression Elicits Trastuzumab Resistance in HER2-Positive Breast Cancer. Clin Cancer Res 23, 636-648 (2017).
33. Dazert, E., et al. Quantitative proteomics and phosphoproteomics on serial tumor biopsies from a sorafenib-treated HCC patient. Proc. Natl. Acad. Sci. U.S.A 113, 1381-1386 (2016).
34. Guo, T., et al. Rapid mass spectrometric conversion of tissue biopsy samples into permanent quantitative digital proteome maps. Nat. Med. 21, 407-413 (2015).
35. Akiyama, T., et al. The transforming potential of the c-erbB-2 protein is regulated by its autophosphorylation at the carboxyl-terminal domain. Mol Cell Biol 11, 833-842 (1991).
36. Chen, C. H., et al. MEK inhibitors induce Akt activation and drug resistance by suppressing negative feedback ERK-mediated HER2 phosphorylation at Thr701. Mol Oncol 11, 1273-1287 (2017).
37. Loi, S., et al. Effects of Estrogen Receptor and Human Epidermal Growth Factor Receptor-2 Levels on the Efficacy of Trastuzumab: A Secondary Analysis of the HERA Trial. JAMA Oncol 2, 1040-1047 (2016).
38. Slamon, D., et al. Adjuvant trastuzumab in HER2-positive breast cancer. N Engl J Med 365, 1273-1283 (2011).
39. Wang, J., et al. TOP2A amplification in breast cancer is a predictive marker of anthracycline-based neoadjuvant chemotherapy efficacy. Breast Cancer Res Treat 135, 531-537 (2012).
40. Gordon, M. A., et al. Synergy between Androgen Receptor Antagonism and Inhibition of mTOR and HER2 in Breast Cancer. Mol Cancer Ther 16, 1389-1400 (2017).
41. Raina, D., et al. Targeting the MUC1-C oncoprotein downregulates HER2 activation and abrogates trastuzumab resistance in breast cancer cells. Oncogene 33, 3422-3431 (2014).
42. Hare, S. H. & Harvey, A. J. mTOR function and therapeutic targeting in breast cancer. Am J Cancer Res 7, 383-404 (2017).
43. Schmid, P., et al. Atezolizumab and Nab-Paclitaxel in Advanced Triple-Negative Breast Cancer. N Engl J Med 379, 2108-2121 (2018).
44. Luen, S. J., et al. Tumour-infiltrating lymphocytes in advanced HER2-positive breast cancer treated with pertuzumab or placebo in addition to trastuzumab and docetaxel: a retrospective analysis of the CLEOPATRA study. Lancet Oncol 18, 52-62 (2017).
45. Hebert, A. S., et al. Comprehensive Single-Shot Proteomics with FAIMS on a Hybrid Orbitrap Mass Spectrometer. Anal Chem 90, 9529-9537 (2018).
46. Pfammatter, S., Bonneil, E., McManus, F. P. & Thibault, P. Accurate Quantitative Proteomic Analyses Using Metabolic Labeling and High Field Asymmetric Waveform Ion Mobility Spectrometry (FAIMS). J Proteome Res 18, 2129-2138 (2019).
47. Schweppe, D. K., et al. Characterization and Optimization of Multiplexed Quantitative Analyses Using High-Field Asymmetric-Waveform Ion Mobility Mass Spectrometry. Anal Chem 91, 4010-4016 (2019).
48. Kuilman, T., et al. CopywriteR: DNA copy number detection from off-target sequence data. Genome Biol. 16, 49 (2015).
49. Aran, D., Hu, Z. & Butte, A. J. xCell: digitally portraying the tissue cellular heterogeneity landscape. Genome Biol 18, 220 (2017).
50. Li, B. & Dewey, C. N. RSEM: accurate transcript quantification from RNA-Seq data with or without a reference genome. BMC Bioinformatics 12, 323 (2011).
51. Mermel, C. H., et al. GISTIC2.0 facilitates sensitive and confident localization of the targets of focal somatic copy-number alteration in human cancers. Genome Biol. 12, R41 (2011).
52. Newman, A. M., et al. Robust enumeration of cell subsets from tissue expression profiles. Nat Methods 12, 453-457 (2015).
53. Yoshihara, K., et al. Inferring tumour purity and stromal and immune cell admixture from expression data. Nat Commun 4, 2612 (2013).

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method to generate DNA, RNA, and protein from a biological sample comprising heterogeneous cells and/or tissue, said method comprising the steps of:
(a) combining into multiple vessels a plurality of sections from different regions of the biological sample, said biological sample optionally being a biopsy;
(b) isolating DNA from the plurality of sections in a first vessel, isolating RNA from the plurality of sections in a second vessel, and isolating protein from the plurality of sections in a third vessel;
wherein the combining step (a) provides a mixture of cells and/or tissues from different regions of the biopsy for the plurality of sections.

2. The method of claim 1, further comprising the step of sectioning the biopsy, and wherein the sectioning and combining step (a) are performed concurrently or separately.

3. The method of claim 1, wherein a plurality of sections from a fourth vessel are used for sample quality analysis.

4. The method of claim 1, wherein the plurality of section for at least one of the 1^{st}, 2^{nd} and/or 3^{rd} vessels comprises nonadjacent sections from the different regions.

5. The method of claim 1, further comprising the steps of analyzing the isolated DNA, analyzing the isolated RNA, and/or analyzing the isolated protein.

6. The method of claim 1, wherein the protein is denatured protein.

7. The method of claim 5, wherein analyzing the isolated DNA, analyzing the isolated RNA, and analyzing the isolated protein determines the status of one or more markers present in cells in the biopsy.

8. The method of claim 7, wherein the one or more markers are cancer markers that are proteins and/or nucleic acids encoding for proteins, said proteins selected from the group consisting of members of the ErbB receptor family, Mucin-1, Mucin-6, PD-1, PD-L1, STAR3, GRB7, mTOR, subunits of mTORC, members of interferon signaling components, AKT, SHC1, EIF4EBP1, TOP2A, and a combination thereof.

9. The method of claim 8, wherein the members of the ErbB receptor family comprise ERBB2, ERBB3, ERBB4, or a combination thereof.

10. The method of claim 5, wherein analyzing the isolated protein further comprises determining the phosphorylation status of the cancer markers.

11. The method of claim 10, wherein the phosphorylation status of the cancer markers is determined by mass spectrometry.

12. The method of claim 5, wherein analyzing the isolated DNA, analyzing the isolated RNA, and analyzing the isolated protein further comprises analyzing the phosphorylation status of HER2.

13. The method of claim 12, wherein analyzing the status of HER2 further comprises analyzing the phosphorylation status of HER2.

14. The method of claim 5, wherein analyzing the isolated DNA, analyzing the isolated RNA, and analyzing the isolated protein determines the status of immune cells in the biopsy.

15. A method of analyzing one or more protein samples from an individual, comprising the steps of:
(a) digesting the protein in each sample into peptides;
(b) tagging the peptides in each sample with a unique tag of known molecular weight to produce tagged peptides;
(c) combining all tagged peptides;
(d) sorting the tagged peptides based on hydrophobicity; and
(e) subjecting the sorted peptides to LC-MS/MS.

16. The method of claim 15, wherein multiple samples from the individual are independently tagged and combined to produce an amount of protein to be digested of at least 250 micrograms.

17. The method of claim 15, wherein step d) comprises sorting the tagged peptides based on hydrophobicity by using basic reverse-phase chromatography.

18. The method of claim 15, wherein prior to subjecting the sorted peptides to LC-MS/MS, the tagged peptides are enriched for phosphopeptides.

19. The method of claim 15, wherein prior to the digesting step the disulfide bonds of the protein are reduced and/or cysteines are alkylated.

20. A method for treating an individual with at least one cancer marker-targeted treatment, the method comprising the steps of:
determining whether the individual has cancer cells positive for the cancer marker by subjecting a biopsy from the individual to the method of claim 1; and one of the following:
(a) administering a therapeutically effective amount of the cancer marker-targeted treatment to the individual who was determined to have cancer cells positive for the marker, or
(b) not administering the cancer marker-targeted treatment to the individual who was determined not to have cancer cells positive for the cancer marker.

21. The method of claim 20, wherein the individual who was determined to have cancer cells positive for the cancer marker is further determined to have cancer cells susceptible to one or more other cancer marker-targeted treatments by:
waiting 1, 2, 3, 4, 5, 6, 7, 8, 9, or 10 or more days after administering the therapeutically effective amount of the cancer marker-targeted treatment;
obtaining a new biological sample from the individual,
repeating the following steps:
(a) combining into multiple vessels a plurality of sections from different regions of the biological sample, said biological sample optionally being a biopsy;
(b) isolating DNA from the plurality of sections in a first vessel, isolating RNA from the plurality of sections in a second vessel, and isolating protein from the plurality of sections in a third vessel;
wherein the combining step (a) provides a mixture of cells and/or tissues from different regions of the biopsy for the plurality of sections; and
determining if the individual has a change in cancer marker status; and one of the following:
(1) continuing the administering of the therapeutically effective amount of the cancer marker-targeted treatment to the individual who was determined to have a change in molecular marker status, or
(2) halting the administering of the therapeutic amount of the cancer marker-targeted treatment to the individual who was determined not to have a change in molecular marker status.

22. The method of claim 21, wherein a therapeutically effective amount of a different cancer therapy is provided to the individual that was determined not to have a change in cancer marker status.

23. A method of distinguishing for an individual cancer marker-positive cells and/or tissue from cancer marker-negative cells and/or tissue, comprising subjecting cells and/or tissue from the individual to the method of claim 1.

24. A method of determining the susceptibility of an individual with cancer to a cancer treatment, comprising the step of subjecting a biological sample from the individual to the method of claim 1.

25. A method of detecting cancer marker status in an individual, said method comprising the steps of:
obtaining a biological sample from the individual;
detecting the cancer marker status in the biological sample by performing the method of claim 1.


SEQUENCE LISTING (66 sequences)

SEQ ID NO: 1 - protein, 19 aa, Artificial Sequence
  Description of Artificial Sequence Synthetic peptide
GDSFTHTPPLDPQELDILK

SEQ ID NO: 2 - protein, 15 aa, Artificial Sequence
  Description of Artificial Sequence Synthetic peptide
GLQSLPTHDPSPLQR

SEQ ID NO: 3 - protein, 18 aa, Artificial Sequence
  Description of Artificial Sequence Synthetic peptide
ESGPGIAPGPEPHGLTNK

SEQ ID NO: 4 - protein, 12 aa, Artificial Sequence
  Description of Artificial Sequence Synthetic peptide
VLGSGAFGTVYK

SEQ ID NO: 5 - protein, 8 aa, Artificial Sequence
  Description of Artificial Sequence Synthetic peptide
DFHMSVVR

SEQ ID NO: 6 - protein, 16 aa, Artificial Sequence
  Description of Artificial Sequence Synthetic peptide
DQLSLGNAALQITDVK

SEQ ID NO: 7 - protein, 11 aa, Artificial Sequence
  Description of Artificial Sequence Synthetic peptide
QGGFLGLSNIK

SEQ ID NO: 8 - protein, 13 aa, Artificial Sequence
  Description of Artificial Sequence Synthetic peptide
TPVAHTTSATSSR

SEQ ID NO: 9 - protein, 15 aa, Artificial Sequence
  Description of Artificial Sequence Synthetic peptide
FTDLLQLVEFHQLNR

SEQ ID NO: 10 - protein, 18 aa, Artificial Sequence
  Description of Artificial Sequence Synthetic peptide
NNEYGDTVYTIEVPFHGK

SEQ ID NO: 11 - protein, 9 aa, Artificial Sequence
  Description of Artificial Sequence Synthetic peptide
TLDQSPELR

SEQ ID NO: 12 - protein, 12 aa, Artificial Sequence
  Description of Artificial Sequence Synthetic peptide
HVDYVADQIVTK

SEQ ID NO: 13 - protein, 9 aa, Artificial Sequence
  Description of Artificial Sequence Synthetic peptide
ESEDKFKLD

SEQ ID NO: 14 - protein, 9 aa, Artificial Sequence
  Description of Artificial Sequence Synthetic peptide
VLLVEQLFR

SEQ ID NO: 15 - protein, 11 aa, Artificial Sequence
  Description of Artificial Sequence Synthetic peptide
VLDQIEFLDTK

SEQ ID NO: 16 - protein, 14 aa, Artificial Sequence
  Description of Artificial Sequence Synthetic peptide
GSDEPPVFLEIHYK

SEQ ID NO: 17 - protein, 15 aa, Artificial Sequence
  Description of Artificial Sequence Synthetic peptide
DVPDLTLIDLPGITR

SEQ ID NO: 18 - protein, 12 aa, Artificial Sequence
  Description of Artificial Sequence Synthetic peptide
VLLEEGSATVPR

SEQ ID NO: 19 - protein, 27 aa, Artificial Sequence
  Description of Artificial Sequence Synthetic peptide
VARPPAAPELGALGSPDLSSLSLAVSR

SEQ ID NO: 20 - protein, 16 aa, Artificial Sequence
  Description of Artificial Sequence Synthetic peptide
SITVNDLTHLLEAAEK

SEQ ID NO: 21 - protein, 13 aa, Artificial Sequence
  Description of Artificial Sequence Synthetic peptide
GLSIEQLTTLAEK

SEQ ID NO: 22 - protein, 14 aa, Artificial Sequence
  Description of Artificial Sequence Synthetic peptide
QVAAVGQEPQVFGR

SEQ ID NO: 23 - protein, 8 aa, Artificial Sequence
  Description of Artificial Sequence Synthetic peptide
EIQDAVAR

SEQ ID NO: 24 - protein, 11 aa, Artificial Sequence
  Description of Artificial Sequence Synthetic peptide
IHHPSLPASGR

SEQ ID NO: 25 - protein, 9 aa, Artificial Sequence
  Description of Artificial Sequence Synthetic peptide
VEALYELLR

SEQ ID NO: 26 - protein, 7 aa, Artificial Sequence
  Description of Artificial Sequence Synthetic peptide
AVPLVNR

SEQ ID NO: 27 - protein, 12 aa, Artificial Sequence
  Description of Artificial Sequence Synthetic peptide
SLPVSVPVWGFK

SEQ ID NO: 28 - protein, 24 aa, Artificial Sequence
  Description of Artificial Sequence Synthetic peptide
SPLAPSEGAGSDVFDGDLGMGAAK

SEQ ID NO: 29 - protein, 24 aa, Artificial Sequence
  Description of Artificial Sequence Synthetic peptide
SPLAPSEGAGSDVFDGDLGMGAAK

SEQ ID NO: 30 - protein, 15 aa, Artificial Sequence
  Description of Artificial Sequence Synthetic peptide
GLQSLPTHDPSPLQR

SEQ ID NO: 31 - protein, 8 aa, Artificial Sequence
  Description of Artificial Sequence Synthetic peptide
PAGATLER

SEQ ID NO: 32 - protein, 42 aa, Artificial Sequence
  Description of Artificial Sequence Synthetic polypeptide
YSEDPTVPLPSETDGYVAPLTCSPQPEYVNQPDVRPQPPSPR

SEQ ID NO: 33 - protein, 17 aa, Artificial Sequence
  Description of Artificial Sequence Synthetic peptide
GTPTAENPEYLGLDVPV

SEQ ID NO: 34 - protein, 12 aa, Artificial Sequence
  Description of Artificial Sequence Synthetic peptide
SLPVSVPVWGFK

SEQ ID NO: 35 - protein, 12 aa, Artificial Sequence
  Description of Artificial Sequence Synthetic peptide
FLMECRNSPVTK

SEQ ID NO: 36 - protein, 8 aa, Artificial Sequence
  Description of Artificial Sequence Synthetic peptide
LNTSDFQK

SEQ ID NO: 37 - protein, 16 aa, Artificial Sequence
  Description of Artificial Sequence Synthetic peptide
ELFDDPSYVNVQNLDK

SEQ ID NO: 38 - protein, 10 aa, Artificial Sequence
  Description of Artificial Sequence Synthetic peptide
IPLENLQIIR

SEQ ID NO: 39 - protein, 7 aa, Artificial Sequence
  Description of Artificial Sequence Synthetic peptide
VLQGLPR

SEQ ID NO: 40 - protein, 18 aa, Artificial Sequence
  Description of Artificial Sequence Synthetic peptide
SLEATDSAFDNPDYWHSR

SEQ ID NO: 41 - protein, 8 aa, Artificial Sequence
  Description of Artificial Sequence Synthetic peptide
EIPDLLEK

SEQ ID NO: 42 - protein, 7 aa, Artificial Sequence
  Description of Artificial Sequence Synthetic peptide
GSADGPR

SEQ ID NO: 43 - protein, 16 aa, Artificial Sequence
  Description of Artificial Sequence Synthetic peptide
AEVIWTSSDHQVLSGK

SEQ ID NO: 44 - protein, 11 aa, Artificial Sequence
  Description of Artificial Sequence Synthetic peptide
NYGQLDIFPAR

SEQ ID NO: 45 - protein, 11 aa, Artificial Sequence
  Description of Artificial Sequence Synthetic peptide
VTNEFVSEEGK

SEQ ID NO: 46 - protein, 15 aa, Artificial Sequence
  Description of Artificial Sequence Synthetic peptide
LIGQQGLVDGLFLVR

SEQ ID NO: 47 - protein, 17 aa, Artificial Sequence
  Description of Artificial Sequence Synthetic peptide
EATAVVDQILAQEENWK

SEQ ID NO: 48 - protein, 17 aa, Artificial Sequence
  Description of Artificial Sequence Synthetic peptide
GYTLADEEEDPLIYQHR

SEQ ID NO: 49 - protein, 15 aa, Artificial Sequence
  Description of Artificial Sequence Synthetic peptide
GFQQISFVNSIATSK

SEQ ID NO: 50 - protein, 7 aa, Artificial Sequence
  Description of Artificial Sequence Synthetic peptide
MAGLAAR

SEQ ID NO: 51 - protein, 25 aa, Artificial Sequence
  Description of Artificial Sequence Synthetic peptide
ENKPVIPLPQILGLTASPGVGGATK

SEQ ID NO: 52 - protein, 12 aa, Artificial Sequence
  Description of Artificial Sequence Synthetic peptide
SAIFHFESAVEK

SEQ ID NO: 53 - protein, 12 aa, Artificial Sequence
  Description of Artificial Sequence Synthetic peptide
LYGSGDQEAWQK

SEQ ID NO: 54 - protein, 9 aa, Artificial Sequence
  Description of Artificial Sequence Synthetic peptide
ELPGFVNYR

SEQ ID NO: 55 - protein, 12 aa, Artificial Sequence
  Description of Artificial Sequence Synthetic peptide
EITFFQTHPYFR

SEQ ID NO: 56 - protein, 15 aa, Artificial Sequence
  Description of Artificial Sequence Synthetic peptide
SAGSILGESSTEASK

SEQ ID NO: 57 - protein, 13 aa, Artificial Sequence
  Description of Artificial Sequence Synthetic peptide
SNFDETYIENVVR

SEQ ID NO: 58 - protein, 14 aa, Artificial Sequence
  Description of Artificial Sequence Synthetic peptide
VVENLQDDFDFNYK

SEQ ID NO: 59 - protein, 15 aa, Artificial Sequence
  Description of Artificial Sequence Synthetic peptide
ELISWGAPGSADSTR

SEQ ID NO: 60 - protein, 20 aa, Artificial Sequence
  Description of Artificial Sequence Synthetic peptide
QPNLPSPGTLAPTTLQGVVK

SEQ ID NO: 61 - protein, 9 aa, Artificial Sequence
  Description of Artificial Sequence Synthetic peptide
FVPLPASAK

SEQ ID NO: 62 - protein, 14 aa, Artificial Sequence
  Description of Artificial Sequence Synthetic peptide
QGLDGPPPTVEELK

SEQ ID NO: 63 - protein, 11 aa, Artificial Sequence
  Description of Artificial Sequence Synthetic peptide
NWNDEWDNLIK

SEQ ID NO: 64 - protein, 12 aa, Artificial Sequence
  Description of Artificial Sequence Synthetic peptide
VLGSGVFGTVHK

SEQ ID NO: 65 - protein, 9 aa, Artificial Sequence
  Description of Artificial Sequence Synthetic peptide
YLVIQGDDR

SEQ ID NO: 66 - protein, 12 aa, Artificial Sequence
  Description of Artificial Sequence Synthetic peptide
YSADPTVFAPER